(19) United States
(12) Patent Application Publication
(10) Pub. No.: US 2020/0115387 A1
(43) Pub. Date: Apr. 16, 2020

(54) ORGANIC SEMICONDUCTORS

(71) Applicant: Merck Patent GmbH, Darmstadt (DE)

(72) Inventors: William MITCHELL, Chandler's Ford (GB); Mansoor D'LAVARI, Southampton (GB); Changsheng WANG, Eastleigh (GB)

(73) Assignee: Merck Patent GmbH, Darmstadt (DE)

(21) Appl. No.: 16/315,759

(22) PCT Filed: Jul. 5, 2017
(86) PCT No.: PCT/EP2017/066739

§ 371 Date: Jan. 7, 2019

(30) Foreign Application Priority Data
Jul. 8, 2016 (EP) 16178592.8

(51) Int. Cl. C07D 495/22; C07F 7/30; C07F 9/00; C07F 7/08; C07D 519/00; H01L 51/00; C07D 517/22

(52) U.S. Cl. CPC C07D 495/22; C07F 7/30; C07F 9/00; C07F 7/0816; C07D 519/00; H01L 51/0069; H01L 51/0074; H01L 51/0073; H01L 51/0072; C07D 517/22; H01L 51/0068; H01L 51/0077

(57) ABSTRACT

The invention relates to novel organic semiconducting compounds containing a polycyclic unit, to methods for their preparation and educts or intermediates used therein, to compositions and formulations containing them, to the use of the compounds and compositions as organic semiconductors in, or for the preparation of, organic electronic (OE) devices, especially organic photovoltaic (OPV) devices, organic photodetectors (OPD), organic field effect transistors (OFET) and organic light emitting diodes (OLED), and to OE devices comprising these compounds or compositions.

### TECHNICAL FIELD

The invention relates to novel organic semiconducting compounds containing a polycyclic unit, to methods for their preparation and educts or intermediates used therein, to compositions and formulations containing them, to the use of the compounds and compositions as organic semiconductors in, or for the preparation of, organic electronic (OE) devices, especially organic photovoltaic (OPV) devices, organic photodetectors (OPD), organic field effect transistors (OFET) and organic light emitting diodes (OLED), and to OE devices comprising these compounds or compositions.

### BACKGROUND

In recent years, there has been development of organic semiconducting (OSC) materials in order to produce more versatile, lower cost electronic devices. Such materials find application in a wide range of devices or apparatus, including organic field effect transistors (OFETs), organic light emitting diodes (OLEDs), organic photodetectors (OPDs), organic photovoltaic (OPV) cells, sensors, memory elements and logic circuits to name just a few. The organic semiconducting materials are typically present in the electronic device in the form of a thin layer, for example of between 50 and 300 nm thickness.

One particular area of importance is organic photovoltaics (OPV). Conjugated polymers have found use in OPVs as they allow devices to be manufactured by solution-processing techniques such as spin casting, dip coating or ink jet printing. Solution processing can be carried out cheaper and on a larger scale compared to the evaporative techniques used to make inorganic thin film devices. Currently, polymer based photovoltaic devices are achieving efficiencies above 10%.

Another particular area of importance are OFETs. The performance of OFET devices is principally based upon the charge carrier mobility of the semiconducting material and the current on/off ratio, so the ideal semiconductor should have a low conductivity in the off state, combined with high charge carrier mobility (>1×10⁻³ cm²V⁻¹s⁻¹). In addition, it is important that the semiconducting material is stable to oxidation i.e. it has a high ionisation potential, as oxidation leads to reduced device performance. Further requirements for the semiconducting material are good processibility, especially for large-scale production of thin layers and desired patterns, and high stability, film uniformity and integrity of the organic semiconductor layer.

The photosensitive layer in an OPV or OPD device is usually composed of at least two materials, a p-type semiconductor, which is typically a conjugated polymer, an oligomer or a defined molecular unit, and an n-type semiconductor, which is typically a fullerene or substituted fullerene, graphene, a metal oxide, or quantum dots.

However, the OSC materials disclosed in prior art for use in OE devices have several drawbacks, such as poor solubility in solvents suitable for mass production, inadequate charge-carrier mobility for commercial application such as in transistors, poor long term stability and non-reproducible film forming properties.

Therefore there is still a need for OSC materials for use in OE devices like OPVs, OPDs and OFETs, which have advantageous properties, in particular good processibility, high solubility in organic solvents, good structural organization and film-forming properties. In addition, the OSC materials should be easy to synthesize, especially by methods suitable for mass production. For use in OPV cells, the OSC materials should especially have a low bandgap, which enables improved light harvesting by the photoactive layer and can lead to higher cell efficiencies, high stability and long lifetime. For use in OFETs the OSC materials should especially have high charge-carrier mobility, high on/off ratio in transistor devices, high oxidative stability and long lifetime.

It was an aim of the present invention to provide new OSC compounds, including p-type and n-type OSCs, which can overcome the drawbacks of the OSCs from prior art, and which provide one or more of the above-mentioned advantageous properties, especially easy synthesis by methods suitable for mass production, good processibility, high stability, long lifetime in OE devices, good solubility in organic solvents, high charge carrier mobility, and a low bandgap. Another aim of the invention was to extend the pool of OSC materials and p-type and n-type OSCs available to the expert. Other aims of the present invention are immediately evident to the expert from the following detailed description.

The inventors of the present invention have found that one or more of the above aims can be achieved by providing compounds as disclosed and claimed hereinafter. These comprise a dithieno[3,2-b:2′,3′-d]thiophene cored polycyclic unit as shown below.

It has been found that compounds comprising such a central polycyclic unit can be used as OSCs which show advantageous properties as described above.

Conjugated polymers based on linearly fused polycyclic aromatic units have been disclosed in prior art for use as p-type OSCs, such as indacenodithiophene (IDT) as disclosed for example in WO 2010/020329 A1 and EP 2075274 A1, or indacenodithienothiophene (IDTT) as disclosed for example in WO 2015/154845 A1.

However, the compounds as disclosed and claimed hereinafter have hitherto not been disclosed in prior art.

### SUMMARY

The invention relates to a compound comprising one or more divalent units of formula I

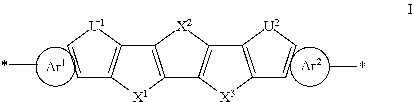

wherein the individual radicals, independently of each other and on each occurrence identically or differently, have the following meanings
Ar^{1,2} arylene or heteroarylene that has from 5 to 20 ring atoms, is mono- or polycyclic, optionally contains fused rings, and is unsubstituted or substituted by one or more identical or different groups L,
X¹, X², X³ O, S or Se,
U¹ CR¹R², SiR¹R², GeR¹R², NR¹ or C═O,
U² CR³R⁴, SiR³R⁴, GeR³R⁴, NR³ or C═O,
R¹⁻⁴ H, F, Cl or straight-chain, branched or cyclic alkyl with 1 to 30, preferably 1 to 20, C atoms, in which one or more CH₂ groups are optionally replaced by —O—, —S—, —C(═O)—, —C(═S)—, —C(═O)—O—, —O—C(═O)—, —NR⁰—, —SiR⁰R⁰⁰—, —CF—, —CR⁰═CR⁰⁰—, —CY¹═CY²— or —C≡C— in such a manner that O and/or S atoms are not linked directly to one another, and in which one or more H atoms are optionally replaced by F, Cl, Br, I or CN, and in which one or more CH₂ or CH₃ groups are optionally replaced by a cationic or anionic group, or aryl, heteroaryl, arylalkyl, heteroarylalkyl, aryloxy or heteroaryloxy, wherein each of the aforementioned cyclic groups has 5 to 20 ring atoms, is mono- or polycyclic, does optionally contain fused rings, and is unsubstituted or substituted by one or more identical or different groups L,
   and the pair of R¹ and R² and/or the pair of R³ and R⁴ together with the C, Si or Ge atom to which they are attached, may also form a spiro group with 5 to 20 ring atoms which is mono- or polycyclic, does optionally contain fused rings, and is unsubstituted or substituted by one or more identical or different groups L,
L F, Cl, —NO₂, —CN, —NC, —NCO, —NCS, —OCN, —SCN, R⁰, OR⁰, SR⁰, —C(═O)X⁰, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO₂R⁰, —OH, —NO₂, —CF₃, —SF₅, or optionally substituted silyl, or carbyl or hydrocarbyl with 1 to 30, preferably 1 to 20, C atoms that is optionally substituted and optionally comprises one or more hetero atoms, preferably F, —CN, R⁰, —OR⁰, —SR⁰, —C(═O)—R⁰, —C(═O)—OR⁰, —O—C(═O)—R⁰, —O—C(═O)—OR⁰, —C(═O)—NHR⁰, or —C(═O)—NR⁰R⁰⁰,
R⁰, R⁰⁰ H or straight-chain or branched alkyl with 1 to 20, preferably 1 to 12, C atoms that is optionally fluorinated,
X⁰ halogen, preferably F or Cl.

The invention further relates to the use of the units of formula I in or as repeating units in conjugated polymers.

A compound comprising one or more units of formula I is hereinafter also referred to as “compound according to the (present) invention”.

The invention further relates to a compound according to the present invention which is a conjugated polymer. The conjugated polymer preferably additionally comprises one or more arylene or heteroarylene units that have from 5 to 20 ring atoms, are mono- or polycyclic, do optionally contain fused rings, are unsubstituted or substituted by one or more identical or different groups L, and are either selected of formula I or are structurally different from formula I, and wherein all the aforementioned units are directly connected to each other. The invention further relates to a conjugated polymer wherein one or more of the additional arylene or heteroarylene units have electron donor property. The invention further relates to a conjugated polymer wherein one or more of the additional arylene or heteroarylene units have electron acceptor property.

The invention further relates to compound according to the present invention which is a small molecule or oligomer.

The invention further relates to a compound according to the present invention which is a monomer comprising a unit of formula I, optionally further comprising one or more additional arylene or heteroarylene units, and further comprising one or more reactive groups which can be reacted to form a conjugated polymer as described above and below.

The invention further relates to a compound according to the present invention which is a small molecule or oligomer comprising one or more units of formula I and further comprising one or more electron-withdrawing groups which can be laterally or terminally attached to the unit of formula I.

The invention further relates to the use of a compound according to the present invention as electron donor or p-type semiconductor, or as electron acceptor or n-type semiconductor.

The invention further relates to the use of a compound according to the present invention as electron donor or electron acceptor component in a semiconducting material, formulation, polymer blend, device or component of a device.

The invention further relates to a semiconducting material, formulation, polymer blend, device or component of a device comprising a compound according to the present invention as electron donor component, and preferably further comprising one or more compounds having electron acceptor properties.

The invention further relates to a semiconducting material, formulation, polymer blend, device or component of a device comprising a compound according to the present invention as electron acceptor component, and preferably further comprising one or more compounds having electron donor properties.

The invention further relates to a composition, which may also be a polymer blend, comprising one or more compounds according to the present invention, and further comprising one or more additional compounds selected from compounds having one or more of semiconducting, charge transport, hole or electron transport, hole or electron blocking, electrically conducting, photoconducting or light emitting properties.

The invention further relates to a composition comprising one or more compounds according to the present invention, and further comprising one or more n-type organic semiconductors, preferably selected from fullerenes or substituted fullerenes.

The invention further relates to a composition comprising a compound according to the present invention, and further comprising one or more electron donors or p-type semiconductors, preferably selected from conjugated polymers.

The invention further relates to a composition comprising a first n-type semiconductor which is a compound according to the present invention, a second n-type semiconductor, which is preferably a fullerene or fullerene derivative, and a p-type semiconductor, which is a conjugated polymer.

The invention further relates to a bulk heterojunction (BHJ) formed from a composition comprising a compound according to the present invention as electron acceptor or n-type semiconductor, and one or more compounds which are electron donor or p-type semiconductors and are preferably selected from conjugated polymers.

The invention further relates to a formulation comprising one or more compounds or a composition according to the present invention, and further comprising one or more solvents, preferably selected from organic solvents.

The invention further relates to an organic semiconducting formulation comprising one or more compounds according to the present invention, and further comprising one or more organic binders or precursors thereof, preferably having a permittivity c at 1,000 Hz and 20° C. of 3.3 or less, and optionally one or more solvents preferably selected from organic solvents.

The invention further relates to an optical, electrooptical, electronic, electroluminescent or photoluminescent device, or a component thereof, or an assembly comprising it, which is prepared using a formulation according to the present invention.

The invention further relates to the use of a compound or composition according to the present invention as semiconducting, charge transport, electrically conducting, photoconducting or light emitting material, or in an optical, electrooptical, electronic, electroluminescent or photoluminescent device, or in a component of such a device or in an assembly comprising such a device or component

The invention further relates to a semiconducting, charge transport, electrically conducting, photoconducting or light emitting material comprising a compound or composition according to the present invention.

The invention further relates to an optical, electrooptical, electronic, electroluminescent or photoluminescent device, or a component thereof, or an assembly comprising it, which comprises a compound or composition according to the present invention, or comprises a semiconducting, charge transport, electrically conducting, photoconducting or light emitting material according to the present invention.

The optical, electrooptical, electronic, electroluminescent and photoluminescent device includes, without limitation, organic field effect transistors (OFET), organic thin film transistors (OTFT), organic light emitting diodes (OLED), organic light emitting transistors (OLET), organic photovoltaic devices (OPV), organic photodetectors (OPD), organic solar cells, dye-sensitized solar cells (DSSC), perovskite-based solar cells, laser diodes, Schottky diodes, photoconductors and photodetectors.

Preferred devices are OFETs, OTFTs, OPVs, OPDs and OLEDs, in particular OTFTs, OPDs and bulk heterojunction (BHJ) OPVs or inverted BHJ OPVs.

Further preferred is the use of a compound or composition according to the present invention as dye in a DSSC or a perovskite-based solar cell. Further preferred is a DSSC or perovskite-based solar cells comprising a compound or composition according to the present invention.

The component of the above devices includes, without limitation, charge injection layers, charge transport layers, interlayers, planarising layers, antistatic films, polymer electrolyte membranes (PEM), conducting substrates and conducting patterns.

The assembly comprising such a device or component includes, without limitation, integrated circuits (IC), radio frequency identification (RFID) tags or security markings or security devices containing them, flat panel displays or backlights thereof, electrophotographic devices, electrophotographic recording devices, organic memory devices, sensor devices, biosensors and biochips.

In addition the compounds, compositions and formulations of the present invention can be used as electrode materials in batteries and in components or devices for detecting and discriminating DNA sequences.

The invention further relates to a bulk heterojunction which comprises, or is being formed from, a composition comprising one or more compounds according to the present invention and one or more n-type organic semiconductors that are preferably selected from fullerenes or substituted fullerenes. The invention further relates to a bulk heterojunction (BHJ) OPV device or inverted BHJ OPV device, comprising such a bulk heterojunction.

### Terms and Definitions

As used herein, the term “polymer” will be understood to mean a molecule of high relative molecular mass, the structure of which essentially comprises multiple repetitions of units derived, actually or conceptually, from molecules of low relative molecular mass (Pure Appl. Chem., 1996, 68, 2291). The term “oligomer” will be understood to mean a molecule of intermediate relative molecular mass, the structure of which essentially comprises a small plurality of units derived, actually or conceptually, from molecules of lower relative molecular mass (Pure Appl. Chem., 1996, 68, 2291). In a preferred meaning as used herein present invention a polymer will be understood to mean a compound having >1, i.e. at least 2 repeat units, preferably ≥5 repeat units, and an oligomer will be understood to mean a compound with >1 and <10, preferably <5, repeat units.

Further, as used herein, the term “polymer” will be understood to mean a molecule that encompasses a backbone (also referred to as “main chain”) of one or more distinct types of repeat units (the smallest constitutional unit of the molecule) and is inclusive of the commonly known terms “oligomer”, “copolymer”, “homopolymer”, “random polymer” and the like. Further, it will be understood that the term polymer is inclusive of, in addition to the polymer itself, residues from initiators, catalysts and other elements attendant to the synthesis of such a polymer, where such residues are understood as not being covalently incorporated thereto. Further, such residues and other elements, while normally removed during post polymerization purification processes, are typically mixed or co-mingled with the polymer such that they generally remain with the polymer when it is transferred between vessels or between solvents or dispersion media.

As used herein, in a formula showing a polymer or a repeat unit, like for example a unit of formula I or a polymer of formula III or IV or their subformulae, an asterisk (*) will be understood to mean a chemical linkage to an adjacent unit or to a terminal group in the polymer backbone. In a ring, like for example a benzene or thiophene ring, an asterisk (*) will be understood to mean a C atom that is fused to an adjacent ring.

As used herein, the terms “repeat unit”, “repeating unit” and “monomeric unit” are used interchangeably and will be understood to mean the constitutional repeating unit (CRU), which is the smallest constitutional unit the repetition of which constitutes a regular macromolecule, a regular oligomer molecule, a regular block or a regular chain (Pure Appl. Chem., 1996, 68, 2291). As further used herein, the term “unit” will be understood to mean a structural unit which can be a repeating unit on its own, or can together with other units form a constitutional repeating unit.

As used herein, a “terminal group” will be understood to mean a group that terminates a polymer backbone. The expression “in terminal position in the backbone” will be understood to mean a divalent unit or repeat unit that is linked at one side to such a terminal group and at the other side to another repeat unit. Such terminal groups include endcap groups, or reactive groups that are attached to a monomer forming the polymer backbone which did not participate in the polymerisation reaction, like for example a group having the meaning of R⁵ or R⁶ as defined below.

As used herein, the term “endcap group” will be understood to mean a group that is attached to, or replacing, a terminal group of the polymer backbone. The endcap group can be introduced into the polymer by an endcapping process. Endcapping can be carried out for example by reacting the terminal groups of the polymer backbone with a monofunctional compound (“endcapper”) like for example an alkyl- or arylhalide, an alkyl- or arylstannane or an alkyl- or arylboronate. The endcapper can be added for example after the polymerisation reaction. Alternatively the endcapper can be added in situ to the reaction mixture before or during the polymerisation reaction. In situ addition of an endcapper can also be used to terminate the polymerisation reaction and thus control the molecular weight of the forming polymer. Typical endcap groups are for example H, phenyl and lower alkyl.

As used herein, the term “small molecule” will be understood to mean a monomeric compound which typically does not contain a reactive group by which it can be reacted to form a polymer, and which is designated to be used in monomeric form. In contrast thereto, the term “monomer” unless stated otherwise will be understood to mean a monomeric compound that carries one or more reactive functional groups by which it can be reacted to form a polymer.

As used herein, the terms “donor” or “donating” and “acceptor” or “accepting” will be understood to mean an electron donor or electron acceptor, respectively. “Electron donor” will be understood to mean a chemical entity that donates electrons to another compound or another group of atoms of a compound. “Electron acceptor” will be understood to mean a chemical entity that accepts electrons transferred to it from another compound or another group of atoms of a compound. See also International Union of Pure and Applied Chemistry, Compendium of Chemical Technology, Gold Book, Version 2.3.2, 19. Aug. 2012, pages 477 and 480.

As used herein, the term “n-type” or “n-type semiconductor” will be understood to mean an extrinsic semiconductor in which the conduction electron density is in excess of the mobile hole density, and the term “p-type” or “p-type semiconductor” will be understood to mean an extrinsic semiconductor in which mobile hole density is in excess of the conduction electron density (see also, J. Thewlis, Concise Dictionary of Physics, Pergamon Press, Oxford, 1973).

As used herein, the term “leaving group” will be understood to mean an atom or group (which may be charged or uncharged) that becomes detached from an atom in what is considered to be the residual or main part of the molecule taking part in a specified reaction (see also Pure Appl. Chem., 1994, 66, 1134).

As used herein, the term “conjugated” will be understood to mean a compound (for example a polymer) that contains mainly C atoms with sp²-hybridisation (or optionally also sp-hybridisation), and wherein these C atoms may also be replaced by hetero atoms. In the simplest case this is for example a compound with alternating C—C single and double (or triple) bonds, but is also inclusive of compounds with aromatic units like for example 1,4-phenylene. The term “mainly” in this connection will be understood to mean that a compound with naturally (spontaneously) occurring defects, or with defects included by design, which may lead to interruption of the conjugation, is still regarded as a conjugated compound.

As used herein, unless stated otherwise the molecular weight is given as the number average molecular weight Mₙ or weight average molecular weight M_{W}, which is determined by gel permeation chromatography (GPC) against polystyrene standards in eluent solvents such as tetrahydrofuran, trichloromethane (TCM, chloroform), chlorobenzene or 1,2,4-trichlorobenzene. Unless stated otherwise, 1,2,4-trichlorobenzene is used as solvent. The degree of polymerization, also referred to as total number of repeat units, n, will be understood to mean the number average degree of polymerization given as n=Mₙ/M_{U}, wherein Mₙ is the number average molecular weight and M_{U} is the molecular weight of the single repeat unit, see J. M. G. Cowie, Polymers: Chemistry & Physics of Modern Materials, Blackie, Glasgow, 1991.

As used herein, the term “carbyl group” will be understood to mean any monovalent or multivalent organic moiety which comprises at least one carbon atom either without any non-carbon atoms (like for example —C≡C—), or optionally combined with at least one non-carbon atom such as B, N, O, S, P, Si, Se, As, Te or Ge (for example carbonyl etc.).

As used herein, the term “hydrocarbyl group” will be understood to mean a carbyl group that does additionally contain one or more H atoms and optionally contains one or more hetero atoms like for example B, N, O, S, P, Si, Se, As, Te or Ge.

As used herein, the term “hetero atom” will be understood to mean an atom in an organic compound that is not a H- or C-atom, and preferably will be understood to mean B, N, O, S, P, Si, Se, As, Te or Ge.

A carbyl or hydrocarbyl group comprising a chain of 3 or more C atoms may be straight-chain, branched and/or cyclic, and may include spiro-connected and/or fused rings.

Preferred carbyl and hydrocarbyl groups include alkyl, alkoxy, thioalkyl, alkylcarbonyl, alkoxycarbonyl, alkylcarbonyloxy and alkoxycarbonyloxy, each of which is optionally substituted and has 1 to 40, preferably 1 to 25, very preferably 1 to 18 C atoms, furthermore optionally substituted aryl or aryloxy having 6 to 40, preferably 6 to 25 C atoms, furthermore alkylaryloxy, arylcarbonyl, aryloxycarbonyl, arylcarbonyloxy and aryloxycarbonyloxy, each of which is optionally substituted and has 6 to 40, preferably 7 to 40 C atoms, wherein all these groups do optionally contain one or more hetero atoms, preferably selected from B, N, O, S, P, Si, Se, As, Te and Ge.

Further preferred carbyl and hydrocarbyl group include for example: a C₁-C₄₀ alkyl group, a C₁-C₄₀ fluoroalkyl group, a C₁-C₄₀ alkoxy or oxaalkyl group, a C₂-C₄₀ alkenyl group, a C₂-C₄₀ alkynyl group, a C₃-C₄₀ allyl group, a C₄-C₄₀ alkyldienyl group, a C₄-C₄₀ polyenyl group, a C₂-C₄₀ ketone group, a C₂-C₄₀ ester group, a C₆-C₁₈ aryl group, a C₆-C₄₀ alkylaryl group, a C₆-C₄₀ arylalkyl group, a C₄-C₄₀ cycloalkyl group, a C₄-C₄₀ cycloalkenyl group, and the like. Preferred among the foregoing groups are a C₁-C₂₀ alkyl group, a C₁-C₂₀ fluoroalkyl group, a C₂-C₂₀ alkenyl group, a C₂-C₂₀ alkynyl group, a C₃-C₂₀ allyl group, a C₄-C₂₀ alkyldienyl group, a C₂-C₂₀ ketone group, a C₂-C₂₀ ester group, a C₆-C₁₂ aryl group, and a C₄-C₂₀ polyenyl group, respectively.

Also included are combinations of groups having carbon atoms and groups having hetero atoms, like e.g. an alkynyl group, preferably ethynyl, that is substituted with a silyl group, preferably a trialkylsilyl group.

The carbyl or hydrocarbyl group may be an acyclic group or a cyclic group. Where the carbyl or hydrocarbyl group is an acyclic group, it may be straight-chain or branched. Where the carbyl or hydrocarbyl group is a cyclic group, it may be a non-aromatic carbocyclic or heterocyclic group, or an aryl or heteroaryl group.

A non-aromatic carbocyclic group as referred to above and below is saturated or unsaturated and preferably has 4 to 30 ring C atoms. A non-aromatic heterocyclic group as referred to above and below preferably has 4 to 30 ring C atoms, wherein one or more of the C ring atoms are optionally replaced by a hetero atom, preferably selected from N, O, S, Si and Se, or by a —S(O)— or —S(O)₂— group. The non-aromatic carbo- and heterocyclic groups are mono- or polycyclic, may also contain fused rings, preferably contain 1, 2, 3 or 4 fused or unfused rings, and are optionally substituted with one or more groups L, wherein

L is selected from F, Cl, —CN, —NC, —NCO, —NCS, —OCN, —SCN, —R⁰, —OR⁰, —SR⁰, —C(═O)X⁰, —C(═O)R⁰, —C(═O)—OR⁰, —O—C(═O)R⁰, —NH₂, —NHR⁰, —NR⁰R⁰⁰, —C(═O)NHR⁰, —C(═O)NR⁰R⁰⁰, —SO₃H, —SO₂R⁰, —OH, —NO₂, —CF₃, —SF₅, or optionally substituted silyl, or carbyl or hydrocarbyl with 1 to 30, preferably 1 to 20, C atoms that is optionally substituted and optionally comprises one or more hetero atoms, wherein X⁰ is halogen, preferably F or Cl, and R⁰, R⁰⁰ denote H or straight-chain or branched alkyl with 1 to 20, preferably 1 to 12 C atoms that is optionally fluorinated.

Preferably L is selected from F, —CN, R, —OR, —SR, —C(═O)—R, —C(═O)—OR, —O—C(═O)—R, —O—C(═O)—OR, —C(═O)—NHR, —C(═O)—NRRⁿ, wherein R and Rⁿ are independently of each other straight-chain or branched alkyl with 1 to 20 C atoms that is optionally fluorinated.

Further preferred substituents L are selected from F or alkyl, alkoxy, oxaalkyl, thioalkyl, fluoroalkyl and fluoroalkoxy with 1 to 16 C atoms, or alkylcarbonyl, alkylcarbonyloxy, alkxoycarbonyl, alkenyl or alkynyl with 2 to 16 C atoms (including the carbonyl-C-atom).

Preferred non-aromatic carbocyclic or heterocyclic groups are tetrahydrofuran, indane, pyran, pyrrolidine, piperidine, cyclopentane, cyclohexane, cycloheptane, cyclopentanone, cyclohexanone, dihydro-furan-2-one, tetrahydro-pyran-2-one and oxepan-2-one.

An aryl group as referred to above and below preferably has 4 to 30 ring C atoms, is mono- or polycyclic and may also contain fused rings, preferably contains 1, 2, 3 or 4 fused or unfused rings, and is optionally substituted with one or more groups L as defined above.

A heteroaryl group as referred to above and below preferably has 4 to 30 ring C atoms, wherein one or more of the C ring atoms are replaced by a hetero atom, preferably selected from N, O, S, Si and Se, is mono- or polycyclic and may also contain fused rings, preferably contains 1, 2, 3 or 4 fused or unfused rings, and is optionally substituted with one or more groups L as defined above.

An arylalkyl or heteroarylalkyl group as referred to above and below preferably denotes —(CH₂)ₐ-aryl or —(CH₂)ₐ-heteroaryl, wherein a is an integer from 1 to 6, preferably 1, and “aryl” and “heteroaryl” have the meanings given above and below. A preferred arylalkyl group is benzyl which is optionally substituted by L.

As used herein, “arylene” will be understood to mean a divalent aryl group, and “heteroarylene” will be understood to mean a divalent heteroaryl group, including all preferred meanings of aryl and heteroaryl as given above and below.

Preferred aryl and heteroaryl groups are phenyl in which, in addition, one or more CH groups may be replaced by N, naphthalene, thiophene, selenophene, thienothiophene, dithienothiophene, fluorene and oxazole, all of which can be unsubstituted, mono- or polysubstituted with L as defined above. Very preferred rings are selected from pyrrole, preferably N-pyrrole, furan, pyridine, preferably 2- or 3-pyridine, pyrimidine, pyridazine, pyrazine, triazole, tetrazole, pyrazole, imidazole, isothiazole, thiazole, thiadiazole, isoxazole, oxazole, oxadiazole, thiophene, preferably 2-thiophene, selenophene, preferably 2-selenophene, thieno[3,2-b]thiophene, thieno[2,3-b]thiophene, furo[3,2-b]furan, furo[2,3-b]furan, seleno[3,2-b]selenophene, seleno[2,3-b]selenophene, thieno[3,2-b]selenophene, thieno[3,2-b]furan, indole, isoindole, benzo[b]furan, benzo[b]thiophene, benzo[1,2-b;4,5-b′]dithiophene, benzo[2,1-b;3,4-b′]dithiophene, quinole, 2-methylquinole, isoquinole, quinoxaline, quinazoline, benzotriazole, benzimidazole, benzothiazole, benzisothiazole, benzisoxazole, benzoxadiazole, benzoxazole, benzothiadiazole, 4H-cyclopenta[2,1-b;3,4-b′]dithiophene, 7H-3,4-dithia-7-sila-cyclopenta[a]pentalene, all of which can be unsubstituted, mono- or polysubstituted with L as defined above. Further examples of aryl and heteroaryl groups are those selected from the groups shown hereinafter.

An alkyl group or an alkoxy group, i.e., where the terminal CH₂ group is replaced by —O—, can be straight-chain or branched. It is preferably straight-chain, has 2, 3, 4, 5, 6, 7, 8, 12, 14, 16 or 18 carbon atoms and accordingly is preferably ethyl, propyl, butyl, pentyl, hexyl, heptyl, octyl, dodecyl, hexadecyl or octadecyl ethoxy, propoxy, butoxy, pentoxy, hexoxy, heptoxy, octoxy, dodecoxy or hexadecoxy, furthermore methyl, nonyl, decyl, undecyl, tridecyl, tetradecyl, pentadecyl, nonoxy, decoxy, undecoxy, tridecoxy or tetradecoxy, for example.

An alkenyl group, i.e., wherein one or more CH₂ groups are replaced by —CH═CH— can be straight-chain or branched. It is preferably straight-chain, has 2 to 10 C atoms and accordingly is preferably vinyl, prop-1-, or prop-2-enyl, but-1-, 2- or but-3-enyl, pent-1-, 2-, 3- or pent-4-enyl, hex-1-, 2-, 3-, 4- or hex-5-enyl, hept-1-, 2-, 3-, 4-, 5- or hept-6-enyl, oct-1-, 2-, 3-, 4-, 5-, 6- or oct-7-enyl, non-1-, 2-, 3-, 4-, 5-, 6-, 7- or non-8-enyl, dec-1-, 2-, 3-, 4-, 5-, 6-, 7-, 8- or dec-9-enyl.

Especially preferred alkenyl groups are C₂-C₇-1E-alkenyl, C₄-C₇-3E-alkenyl, C₅-C₇-4-alkenyl, C₆-C₇-5-alkenyl and C₇-6-alkenyl, in particular C₂-C₇-1E-alkenyl, C₄-C₇-3E-alkenyl and C₅-C₇-4-alkenyl. Examples for particularly preferred alkenyl groups are vinyl, 1E-propenyl, 1E-butenyl, 1E-pentenyl, 1E-hexenyl, 1E-heptenyl, 3-butenyl, 3E-pentenyl, 3E-hexenyl, 3E-heptenyl, 4-pentenyl, 4Z-hexenyl, 4E-hexenyl, 4Z-heptenyl, 5-hexenyl, 6-heptenyl and the like. Groups having up to 5 C atoms are generally preferred.

An oxaalkyl group, i.e., where one CH₂ group is replaced by —O—, is preferably straight-chain 2-oxapropyl (=methoxymethyl), 2-(=ethoxymethyl) or 3-oxabutyl (=2-methoxyethyl), 2-, 3-, or 4-oxapentyl, 2-, 3-, 4-, or 5-oxahexyl, 2-, 3-, 4-, 5-, or 6-oxaheptyl, 2-, 3-, 4-, 5-, 6- or 7-oxaoctyl, 2-, 3-, 4-, 5-, 6-, 7- or 8-oxanonyl or 2-, 3-, 4-, 5-, 6-, 7-, 8- or 9-oxadecyl, for example.

In an alkyl group wherein one CH₂ group is replaced by —O— and one CH₂ group is replaced by —C(O)—, these radicals are preferably neighboured. Accordingly these radicals together form a carbonyloxy group —C(O)—O— or an oxycarbonyl group —O—C(O)—. Preferably this group is straight-chain and has 2 to 6 C atoms. It is accordingly preferably acetyloxy, propionyloxy, butyryloxy, pentanoyloxy, hexanoyloxy, acetyloxymethyl, propionyloxymethyl, butyryloxymethyl, pentanoyloxymethyl, 2-acetyloxyethyl, 2-propionyloxyethyl, 2-butyryloxyethyl, 3-acetyloxypropyl, 3-propionyloxypropyl, 4-acetyloxybutyl, methoxycarbonyl, ethoxycarbonyl, propoxycarbonyl, butoxycarbonyl, pentoxycarbonyl, methoxycarbonylmethyl, ethoxycarbonylmethyl, propoxycarbonylmethyl, butoxycarbonylmethyl, 2-(methoxycarbonyl)ethyl, 2-(ethoxycarbonyl)ethyl, 2-(propoxycarbonyl)ethyl, 3-(methoxycarbonyl)propyl, 3-(ethoxycarbonyl)propyl, 4-(methoxycarbonyl)-butyl.

An alkyl group wherein two or more CH₂ groups are replaced by —O— and/or —C(O)O— can be straight-chain or branched. It is preferably straight-chain and has 3 to 12 C atoms. Accordingly it is preferably bis-carboxy-methyl, 2,2-bis-carboxy-ethyl, 3,3-bis-carboxy-propyl, 4,4-bis-carboxy-butyl, 5,5-bis-carboxy-pentyl, 6,6-bis-carboxy-hexyl, 7,7-bis-carboxy-heptyl, 8,8-bis-carboxy-octyl, 9,9-bis-carboxy-nonyl, 10,10-bis-carboxy-decyl, bis-(methoxycarbonyl)-methyl, 2,2-bis-(methoxycarbonyl)-ethyl, 3,3-bis-(methoxycarbonyl)-propyl, 4,4-bis-(methoxycarbonyl)-butyl, 5,5-bis-(methoxycarbonyl)-pentyl, 6,6-bis-(methoxycarbonyl)-hexyl, 7,7-bis-(methoxycarbonyl)-heptyl, 8,8-bis-(methoxycarbonyl)-octyl, bis-(ethoxycarbonyl)-methyl, 2,2-bis-(ethoxycarbonyl)-ethyl, 3,3-bis-(ethoxycarbonyl)-propyl, 4,4-bis-(ethoxycarbonyl)-butyl, 5,5-bis-(ethoxycarbonyl)-hexyl.

A thioalkyl group, i.e., where one CH₂ group is replaced by —S—, is preferably straight-chain thiomethyl (—SCH₃), 1-thioethyl (—SCH₂CH₃), 1-thiopropyl (═—SCH₂CH₂CH₃), 1-(thiobutyl), 1-(thiopentyl), 1-(thiohexyl), 1-(thioheptyl), 1-(thiooctyl), 1-(thiononyl), 1-(thiodecyl), 1-(thioundecyl) or 1-(thiododecyl), wherein preferably the CH₂ group adjacent to the sp² hybridised vinyl carbon atom is replaced.

A fluoroalkyl group is perfluoroalkyl CᵢF₂ᵢ₊₁, wherein i is an integer from 1 to 15, in particular CF₃, C₂F₅, C₃F₇, C₄F₉, C₅F₁₁, C₆F₁₃, C₇F₁₅ or C₈F₁₇, very preferably C₆F₁₃, or partially fluorinated alkyl, preferably with 1 to 15 C atoms, in particular 1,1-difluoroalkyl, all of the aforementioned being straight-chain or branched.

Preferably “fluoroalkyl” means a partially fluorinated (i.e. not perfluorinated) alkyl group.

Alkyl, alkoxy, alkenyl, oxaalkyl, thioalkyl, carbonyl and carbonyloxy groups can be achiral or chiral groups. Particularly preferred chiral groups are 2-butyl (=1-methylpropyl), 2-methylbutyl, 2-methylpentyl, 3-methylpentyl, 2-ethylhexyl, 2-butyloctyl, 2-hexyldecyl, 2-octyldodecyl, 2-propylpentyl, in particular 2-methylbutyl, 2-methylbutoxy, 2-methylpentoxy, 3-methyl-pentoxy, 2-ethyl-hexoxy, 2-butyloctoxyo, 2-hexyldecoxy, 2-octyldodecoxy, 1-methylhexoxy, 2-octyloxy, 2-oxa-3-methylbutyl, 3-oxa-4-methyl-pentyl, 4-methylhexyl, 2-hexyl, 2-octyl, 2-nonyl, 2-decyl, 2-dodecyl, 6-methoxy-octoxy, 6-methyloctoxy, 6-methyloctanoyloxy, 5-methylheptyloxy-carbonyl, 2-methylbutyryloxy, 3-methylvaleroyloxy, 4-methylhexanoyloxy, 2-chloro-propionyloxy, 2-chloro-3-methylbutyryloxy, 2-chloro-4-methyl-valeryl-oxy, 2-chloro-3-methylvaleryloxy, 2-methyl-3-oxapentyl, 2-methyl-3-oxa-hexyl, 1-methoxypropyl-2-oxy, 1-ethoxypropyl-2-oxy, 1-propoxypropyl-2-oxy, 1-butoxypropyl-2-oxy, 2-fluorooctyloxy, 2-fluorodecyloxy, 1,1,1-trifluoro-2-octyloxy, 1,1,1-trifluoro-2-octyl, 2-fluoromethyloctyloxy for example. Very preferred are 2-ethylhexyl, 2-butyloctyl, 2-hexyldecyl, 2-octyldodecyl, 2-hexyl, 2-octyl, 2-octyloxy, 1,1,1-trifluoro-2-hexyl, 1,1,1-trifluoro-2-octyl and 1,1,1-trifluoro-2-octyloxy.

Preferred achiral branched groups are isopropyl, isobutyl (=methylpropyl), isopentyl (=3-methylbutyl), tert. butyl, isopropoxy, 2-methyl-propoxy and 3-methylbutoxy.

In a preferred embodiment, the alkyl groups are independently of each other selected from primary, secondary or tertiary alkyl or alkoxy with 1 to 30 C atoms, wherein one or more H atoms are optionally replaced by F, or aryl, aryloxy, heteroaryl or heteroaryloxy that is optionally alkylated or alkoxylated and has 4 to 30 ring atoms. Very preferred groups of this type are selected from the group consisting of the following formulae

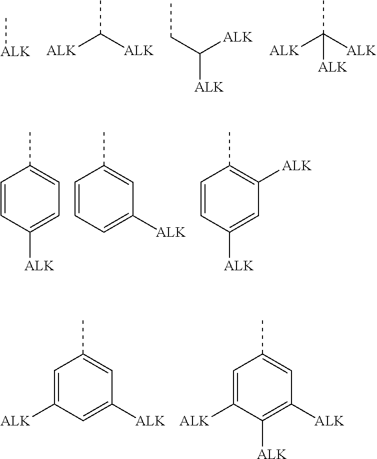

wherein “ALK” denotes optionally fluorinated, preferably linear, alkyl or alkoxy with 1 to 20, preferably 1 to 16 C-atoms, in case of tertiary groups very preferably 1 to 9 C atoms, and the dashed line denotes the link to the ring to which these groups are attached. Especially preferred among these groups are those wherein all ALK subgroups are identical.

As used herein, if an aryl(oxy) or heteroaryl(oxy) group is “alkylated or alkoxylated”, this means that it is substituted with one or more alkyl or alkoxy groups having from 1 to 20 C-atoms and being straight-chain or branched and wherein one or more H atoms are optionally substituted by an F atom.

Above and below, Y¹ and Y² are independently of each other H, F, Cl or CN.

As used herein, —CO—, —C(═O)— and —C(O)— will be understood to mean a carbonyl group, i.e. a group having the structure

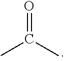

As used herein, C═CR¹R² will be understood to mean a group having the structure

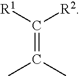

As used herein, “halogen” includes F, Cl, Br or I, preferably F, Cl or Br. A halogen atom that represents a substituent on a ring or chain is preferably F or Cl, very preferably F. A halogen atom that represents a reactive group in a monomer is preferably Cl, Br or I, very preferably Br or I.

### DETAILED DESCRIPTION

The compounds of the present invention are easy to synthesize and exhibit advantageous properties. They show good processability for the device manufacture process, high solubility in organic solvents, and are especially suitable for large scale production using solution processing methods.

Co-polymers derived from monomers of the present invention and electron acceptor monomers show low bandgaps, high charge carrier mobilities, high external quantum efficiencies in BHJ solar cells, good morphology when used in p/n-type blends e.g. with fullerenes, high oxidative stability, a long lifetime in electronic devices, and are promising materials for organic electronic OE devices, especially for OPV devices with high power conversion efficiency.

The compounds of the present invention are especially suitable as p-type semiconductors for the preparation of blends of p-type and n-type semiconductors which are suitable for use in BHJ photovoltaic devices.

Besides, the compounds of the present invention show the following advantageous properties:
i) Additional solubility can be introduced into the compound by inclusion of various solubilizing groups in R¹⁻¹¹ positions.
ii) Additional fine-tuning of the electronic energies (HOMO/LUMO levels) by co-polymerisation with appropriate co-monomer(s) can afford attractive candidate materials for OPV and/or OPD applications.
iii) The polycyclic units of formula I have planar structures that enable strong pi-pi stacking in the solid state leading to improved charge transport properties in the form of higher charge carrier mobility.
iv) An incorporation of various substituents into the poylcyclic core results in monomers with either electron donating property, like for example dialkyl-substituted core, or electron accepting property, like for example dialkyl ester-substituted core.
v) The unit of formula I can thus be altered from a donor unit to an acceptor unit by switching the nature of the solubilising groups, for example from electron donating groups like alkyl or alkoxy to electron withdrawing groups like ketone or ester, thus allowing a much broader applicability of the core for use in OPV or OPD polymers
vi) Using both units of formula I substituted by electron donating groups like alkyl or alkoxy and units of formula I substituted by electron withdrawing groups like ketone or ester, it is possible to create a donor-acceptor polymer from units of formula I, optionally with spacer units like thiophene, dithiophene or thienothiophene to keep the backbone flat, but without further donor or acceptor units. Such polymers are expected to have a high Voc.

In the units of formula I and its subformulae Ar¹ and Ar² are preferably selected from the following formulae:

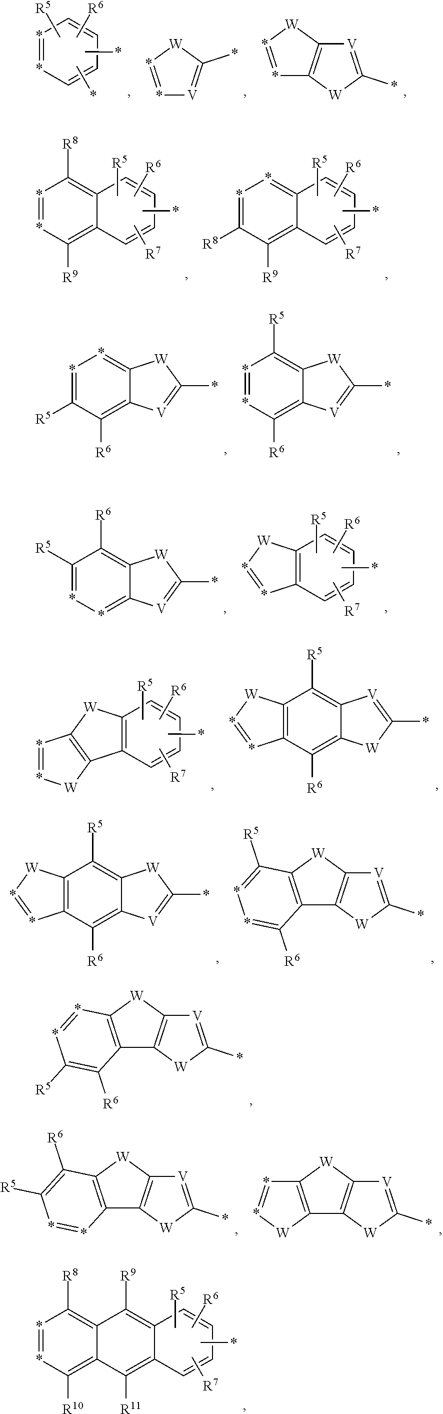

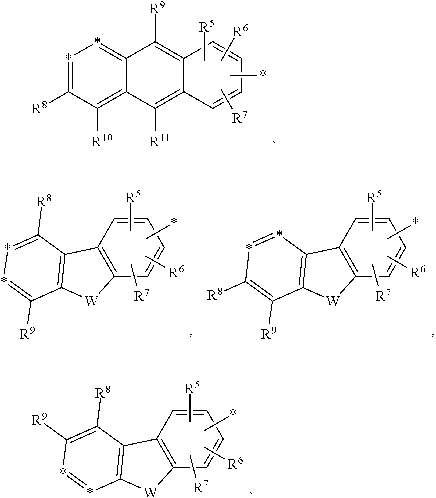

wherein the individual radicals, independently of each other and on each occurrence identically or differently, have the following meanings
W S, O or Se,
V Ca¹ or N,
R⁵⁻¹¹ H, F, Cl or straight-chain, branched or cyclic alkyl with 1 to 30, preferably 1 to 20, C atoms, in which one or more CH₂ groups are optionally replaced by —O—, —S—, —C(═O)—, —C(═S)—, —C(═O)—O—, —O—C(═O)—, —NR⁰—, —SiR⁰R⁰⁰—, —CF₂—, —CR⁰═CR⁰⁰—, —CY¹═CY²— or —C≡C— in such a manner that O and/or S atoms are not linked directly to one another, and in which one or more H atoms are optionally replaced by F, Cl, Br, I or CN, and in which one or more CH₂ or CH₃ groups are optionally replaced by a cationic or anionic group, or aryl, heteroaryl, arylalkyl, heteroarylalkyl, aryloxy or heteroaryloxy, wherein each of the aforementioned cyclic groups has 5 to 20 ring atoms, is mono- or polycyclic, does optionally contain fused rings, and is unsubstituted or substituted by one or more identical or different groups L as defined in formula I,

Very preferably Ar¹ and Ar² are selected from the following formulae:

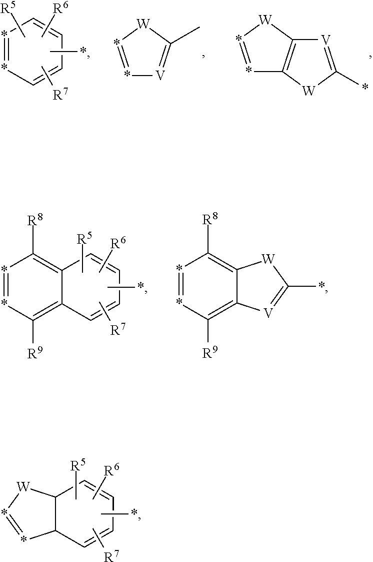

wherein W, V and R⁵⁻⁹ have the meanings given above and below, and wherein W is preferably S and V is preferably CH.

Preferred units of formula I are selected from the following subformulae

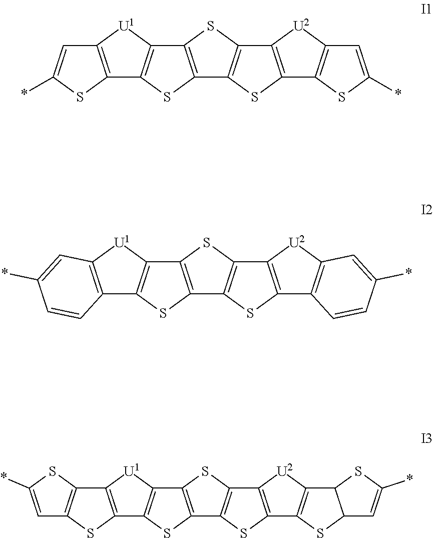

wherein U^{1,2} are as defined in formula I, and preferably U¹ and U² denote CR¹R² and CR³R⁴ respectively.

In the units of formula I and its subformulae preferably R¹⁻⁴ are different from H.

Preferably R¹⁻⁴ are selected from the following groups or any combination thereof:
the group consisting of R, —OR and —SR wherein R is straight-chain or branched alkyl with 1 to 20 C atoms which is unsubstituted or substituted by one or more F atoms,
the group consisting of —C(═O)—R, —C(═O)—OR, —OC(═O)—R, —C(═O)—NHR and —C(═O)—NRRⁿ, wherein R and Rⁿ are independently of each other straight-chain or branched alkyl with 1 to 20 C atoms that is optionally fluorinated,
the group consisting of aryl, aryloxy, heteroaryl and heteroaryloxy, each of which has 4 to 20 ring atoms and optionally contains fused rings and is unsubstituted or substituted by one or more groups L as defined in formula I.

If R¹⁻⁴ denote an aryl(oxy) or heteroaryl(oxy) group, it is preferably selected from phenyl, naphthyl, phenanthryl, anthracenyl, indenyl, pyrrole, furan, pyridine, thiazole, thiophene, thieno[3,2-b]thiophene or thieno[2,3-b]thiophene, each of which is unsubstituted or substituted with one or more groups L, preferably with one or more groups selected from F and alkyl, alkoxy or thioalkyl each having from 1 to 20 C atoms and being optionally fluorinated.

Very preferred groups R¹⁻⁴ are selected from the group consisting of phenyl, 4-biphenyl, 2-indenyl, 1- or 2-naphthyl, 1-, 2- or 3-phenanthrenyl and 1-, 2- or 9-anthracenyl, all of which are optionally substituted by one or more groups R⁵ which are preferably selected from straight-chain or branched alkyl or alkoxy with 1 to 20 C atoms that is optionally fluorinated.

Most preferred groups R¹⁻⁴ in the units of formula I and its subformulae denote phenyl that is substituted in 4-position by a group R⁵ as defined above.

Preferably R⁵⁻¹¹, if being different from H, are selected from the following groups or any combination thereof:
the group consisting of R, —OR and —SR wherein R is straight-chain or branched alkyl with 1 to 20 C atoms which is unsubstituted or substituted by one or more F atoms,
the group consisting of —C(═O)—R, —C(═O)—OR, —OC(═O)—R, —C(═O)—NHR and —C(═O)—NRRⁿ, wherein R and Rⁿ are independently of each other straight-chain or branched alkyl with 1 to 20 C atoms that is optionally fluorinated,
the group consisting of aryl, aryloxy, heteroaryl and heteroaryloxy, each of which has 4 to 20 ring atoms and optionally contains fused rings and is unsubstituted or substituted by one or more groups L as defined in formula I, preferably.

If R⁵⁻¹¹ denote an aryl(oxy) or heteroaryl(oxy) group, it is preferably selected from phenyl, naphthyl, phenanthryl, anthracenyl, indenyl, pyrrole, furan, pyridine, thiazole, thiophene, thieno[3,2-b]thiophene or thieno[2,3-b]thiophene, each of which is unsubstituted or substituted with one or more groups L, preferably with one or more groups selected from F and alkyl, alkoxy or thioalkyl each having from 1 to 20 C atoms and being optionally fluorinated.

In another preferred embodiment of the present invention one or more of R¹⁻¹¹ denote straight-chain, branched or cyclic alkyl with 1 to 20 C-atoms wherein one or more CH₂ or CH₃ groups are substituted by a cationic or anionic group.

The cationic group is preferably selected from the group consisting of phosphonium, sulfonium, ammonium, uronium, thiouronium, guanidinium or heterocyclic cations such as imidazolium, pyridinium, pyrrolidinium, triazolium, morpholinium or piperidinium cation.

Preferred cationic groups are selected from the group consisting of tetraalkylammonium, tetraalkylphosphonium, N-alkylpyridinium, N,N-dialkylpyrrolidinium, 1,3-dialkylimidazolium, wherein “alkyl” preferably denotes a straight-chain or branched alkyl group with 1 to 12 C atoms.

Further preferred cationic groups are selected from the group consisting of the following formulae

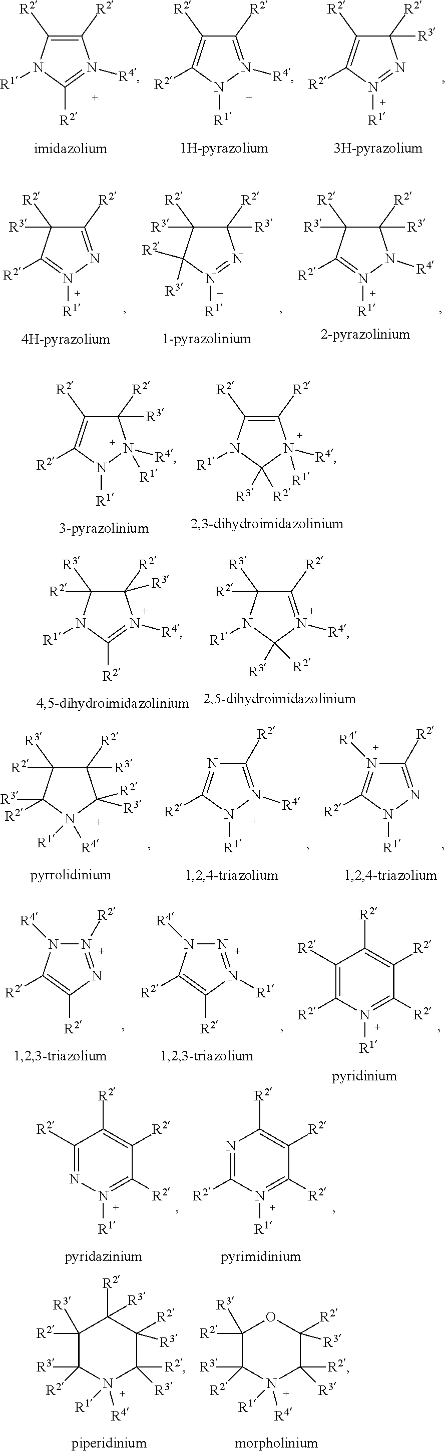

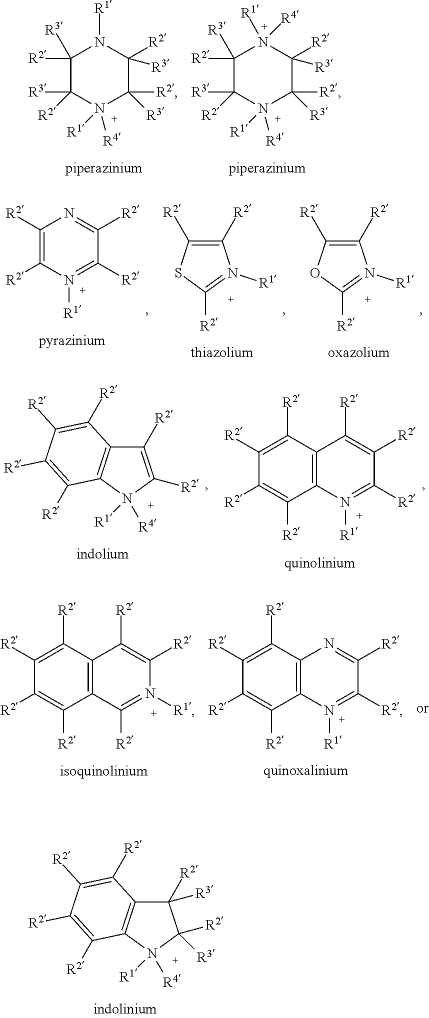

wherein R^{1′}, R^{2′}, R^{3′} and R^{4′} denote, independently of each other, H, a straight-chain or branched alkyl group with 1 to 12 C atoms or non-aromatic carbo- or heterocyclic group or an aryl or heteroaryl group, each of the aforementioned groups having 3 to 20, preferably 5 to 15, ring atoms, being mono- or polycyclic, and optionally being substituted by one or more identical or different substituents L as defined below, or denote a link to the respective group R¹⁻⁴.

In the above cationic groups of the above-mentioned formulae any one of the groups R^{1′}, R^{2′}, R^{3′} and R^{4′} (if they replace a CH₃ group) can denote a link to the group R¹, or two neighbored groups R^{1′}, R^{2′}, R^{3′} or R^{4′} (if they replace a CH₂ group) can denote a link to the respective group R¹⁻⁴.

The anionic group is preferably selected from the group consisting of borate, imide, phosphate, sulfonate, sulfate, succinate, naphthenate or carboxylate, very preferably from phosphate, sulfonate or carboxylate.

The compounds according to the present invention include small molecules, monomers, oligomers and polymers.

A preferred embodiment of the present invention relates to a conjugated polymer comprising, preferably consisting of, one or more repeating units of formula II1 and/or II2, and optionally one or more repeating units of formula II3:

—(Ar¹)ₐ—U—(Ar²)_{b}—(Ar³)_{c}—(Ar⁴)_{d}— II1

—(Ar¹)ₐ—(Ar²)_{b}—U—(Ar³)_{c}—(Ar⁴)_{d}— II2

—(Ar¹)ₐ—(Ar²)_{b}—(Ar³)_{c}—(Ar⁴)_{d}—II3

wherein the individual radicals, independently of each other and on each occurrence identically or differently, have the following meanings
U a unit of formula I or its subformulae as defined above and below,
Ar¹⁻⁴ arylene or heteroarylene that has 5 to 20 ring atoms, is mono- or polycyclic, does optionally contain fused rings, is unsubstituted or substituted by one or more identical or different groups L, and is different from U,
a, b, c, d 0 or 1, wherein in formula II3 a+b+c+d≥1.

Preferably the conjugated polymer comprises one or more repeating units of formula II1 or II2 wherein a+b+c+c≥1.

Further preferably the conjugated polymer comprises one or more repeating units of formula II1 wherein b=1 and a=c=d=0 and one or more repeating units of formula II3 wherein a=b=1 and c=d=0.

Further preferably the conjugated polymer comprises two or more distinct repeating units of formula UU1 wherein b=1 and a=c=d=0.

Further preferably at least one of Ar¹, Ar², Ar³ and Ar⁴ is an arylene or heteroarylene group as being defined in formula II1 and having electron acceptor property.

Preferably L denotes F or is selected from the following groups
the group consisting of R, —OR and —SR wherein R is straight-chain or branched alkyl with 1 to 20 C atoms which is unsubstituted or substituted by one or more F atoms,
the group consisting of —C(═O)—R, —C(═O)—OR, —OC(═O)—R, —C(═O)—NHR and —C(═O)—NRRⁿ, wherein R and Rⁿ are independently of each other straight-chain or branched alkyl with 1 to 20 C atoms that is optionally fluorinated,

Further preferably the conjugated polymer according to the present invention is selected of formula III:

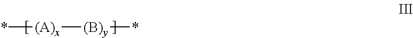

wherein
A is a unit of formula I, II1 or II2 as defined above and below,
B is a unit of formula I, II1, II2 or II3 as defined above and below,
x is >0 and ≤1,
y is ≥0 and <1,
x+y is 1, and
n is an integer ≥5.

Preferred polymers of formula III are selected from the following subformulae

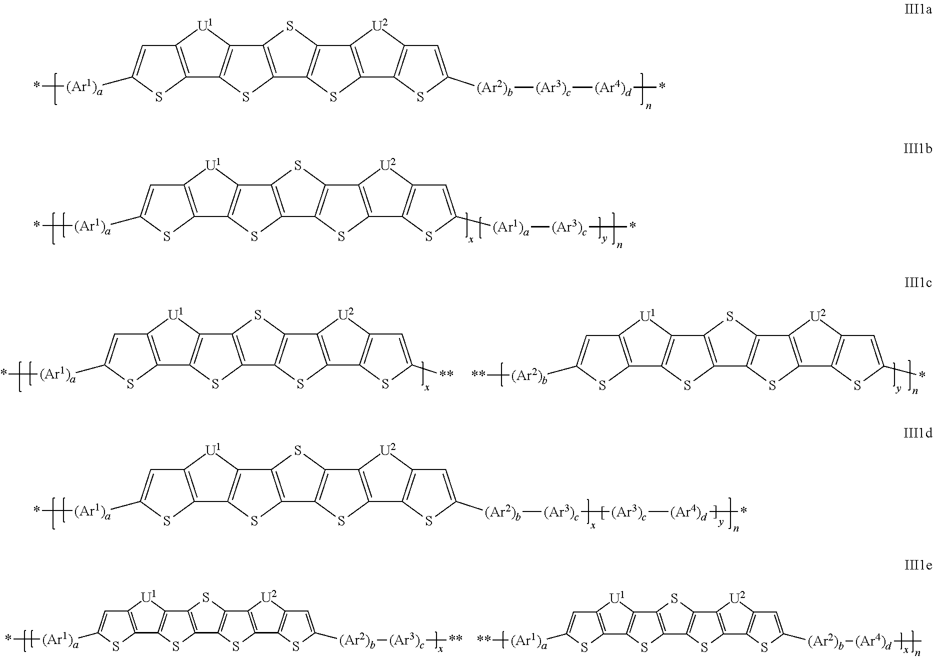

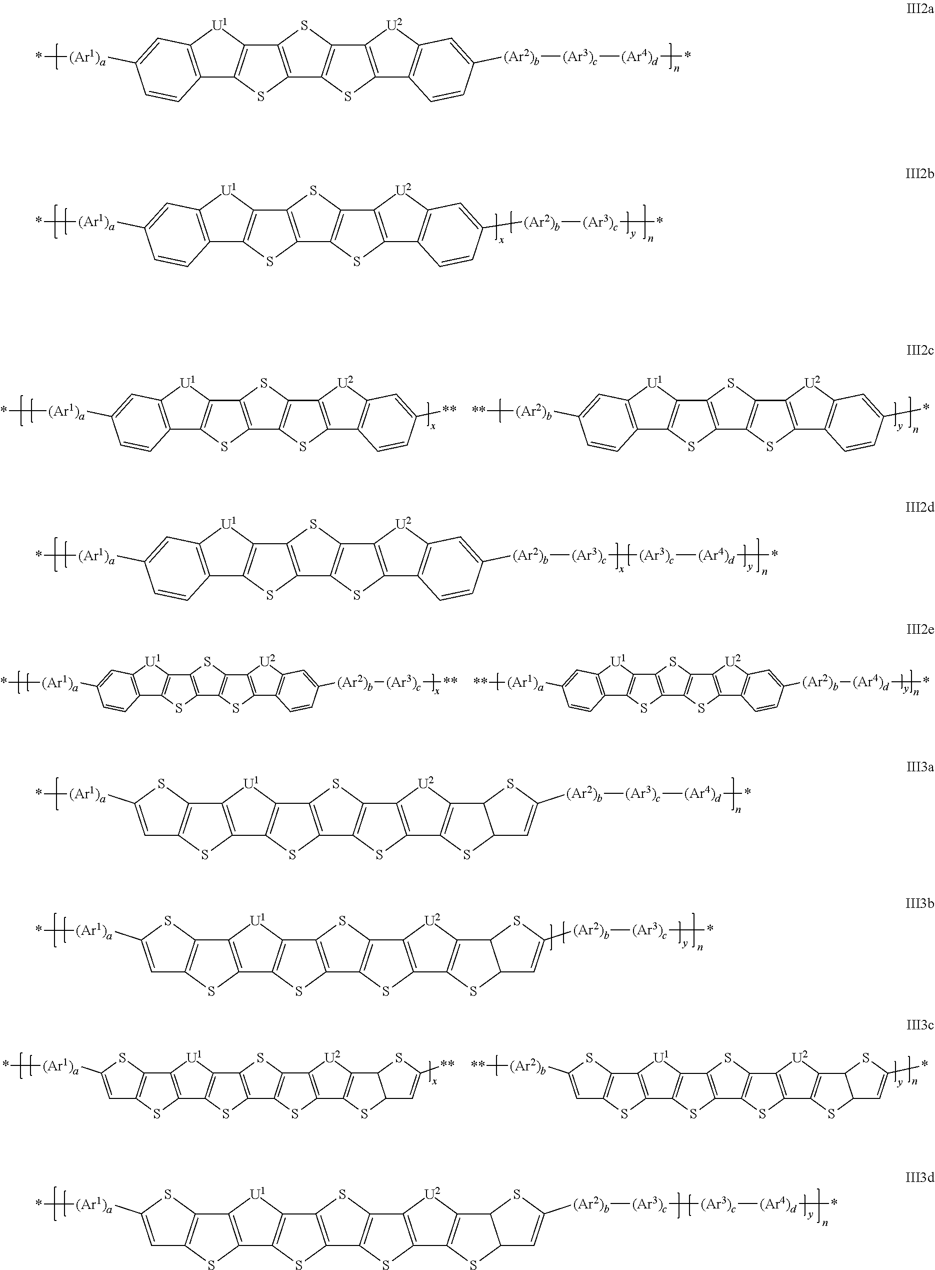

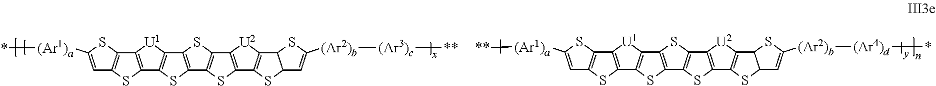

wherein U¹⁻² have the meanings of formula I or one of the preferred meanings given above and below, Ar¹⁻⁴ and a-d have the meanings of formula Ill or one of the preferred meanings given above and below, and x, y and n have the meanings of formula III or one of the preferred meanings given above and below.

In the polymers of formula III and its subformulae, x and y denote the mole fraction of repeating units A and B, respectively, and n denotes the degree of polymerisation or total number of repeating units A and B. These formulae include block copolymers, random or statistical copolymers and alternating copolymers of A and B, as well as homopolymers of A for the case when x>0 and y=0.

In the polymers of formula III and its subformulae, x is preferably from 0.1 to 0.9, very preferably from 0.3 to 0.7.

In the polymers of formula III and its subformulae, y is preferably from 0.1 to 0.9, very preferably from 0.3 to 0.7.

In the polymers according to the present invention, the total number of repeating units n is preferably from 2 to 10,000. The total number of repeating units n is preferably ≥5, very preferably ≥10, most preferably ≥50, and preferably ≤500, very preferably ≤1,000, most preferably ≤2,000, including any combination of the aforementioned lower and upper limits of n.

The polymers of the present invention include homopolymers and copolymers, like statistical or random copolymers, alternating copolymers and block copolymers, as well as combinations thereof.

Further preferably the conjugated polymer is selected of formula IV

R²¹-chain-R²² IV

wherein “chain” denotes a polymer chain selected of formulae III and III1a-III3e, and R²¹ and R²² have independently of each other one of the meanings of L as defined above, or denote, independently of each other, H, F, Br, Cl, I, —CH₂Cl, —CHO, —CR′═CR″₂, —SiR′R″R′″, —SiR′X′X″, —SiR′R″X′, —SnR′R″R′″, —BR′R″, —B(OR′)(OR″), —B(OH)₂, —O—SO₂—R′, —C≡CH, —C≡C—SiR′₃, —ZnX′ or an endcap group, X′ and X″ denote halogen, R′, R″ and R′″ have independently of each other one of the meanings of R⁰ given in formula I, and preferably denote alkyl with 1 to 12 C atoms, and two of R′, R″ and R′″ may also form a cyclosilyl, cyclostannyl, cycloborane or cycloboronate group with 2 to 20 C atoms together with the respective hetero atom to which they are attached.

Preferred endcap groups R²¹ and R²² are H, C₁₋₂₀ alkyl, or optionally substituted C₆₋₁₂ aryl or C₂₋₁₀ heteroaryl, very preferably H or phenyl.

Especially preferred are repeating units and polymers of formulae II1, II2, III, III1a-III3e, IV and their subformulae wherein one or more of Ar¹, Ar², Ar³ and Ar⁴ denote arylene or heteroarylene, preferably having electron donor properties, selected from the group consisting of the following formulae

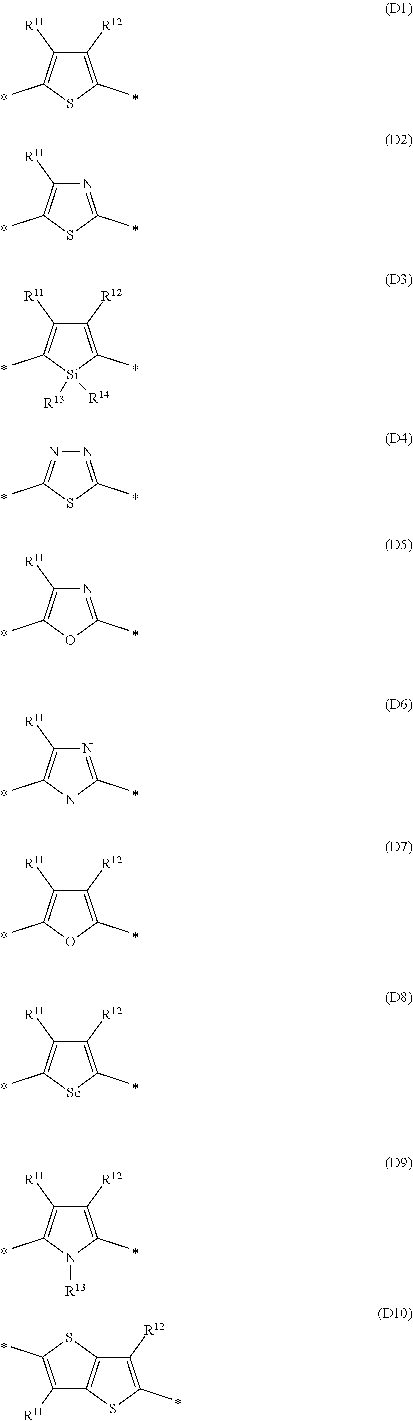

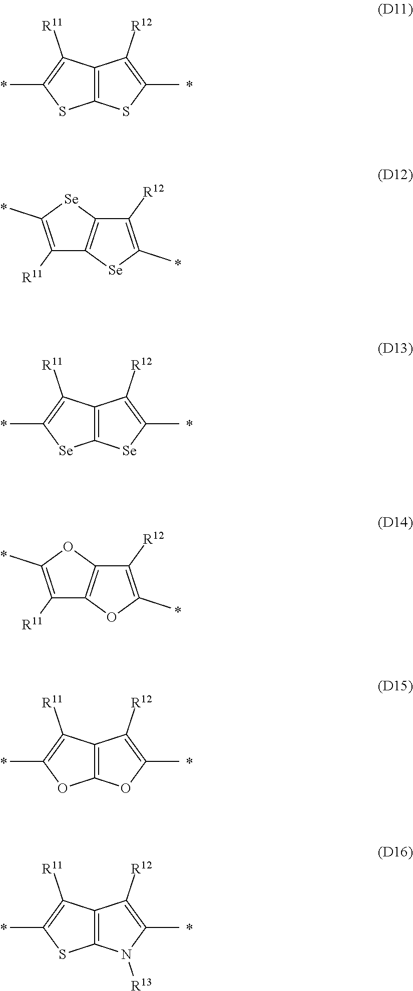

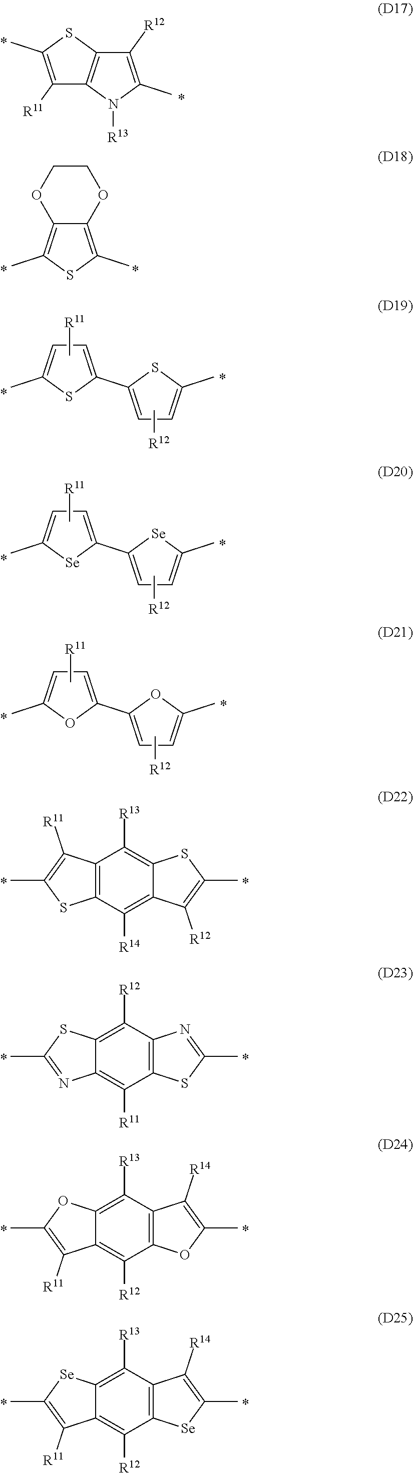

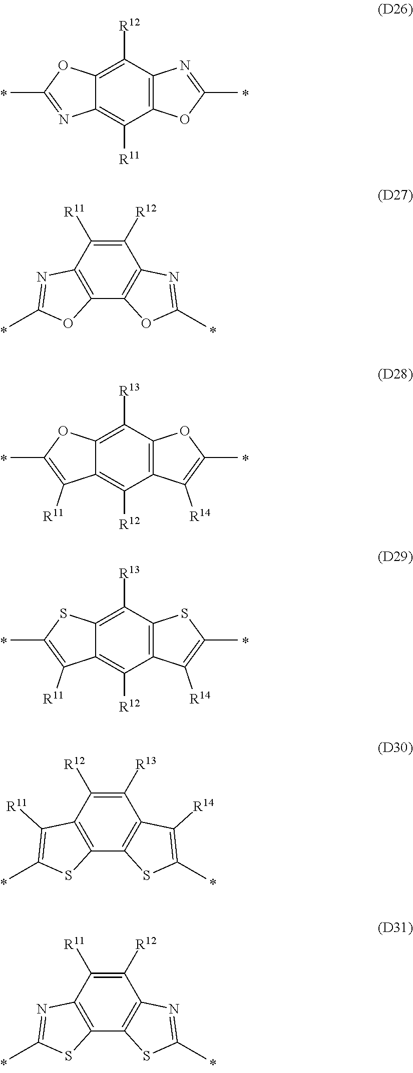

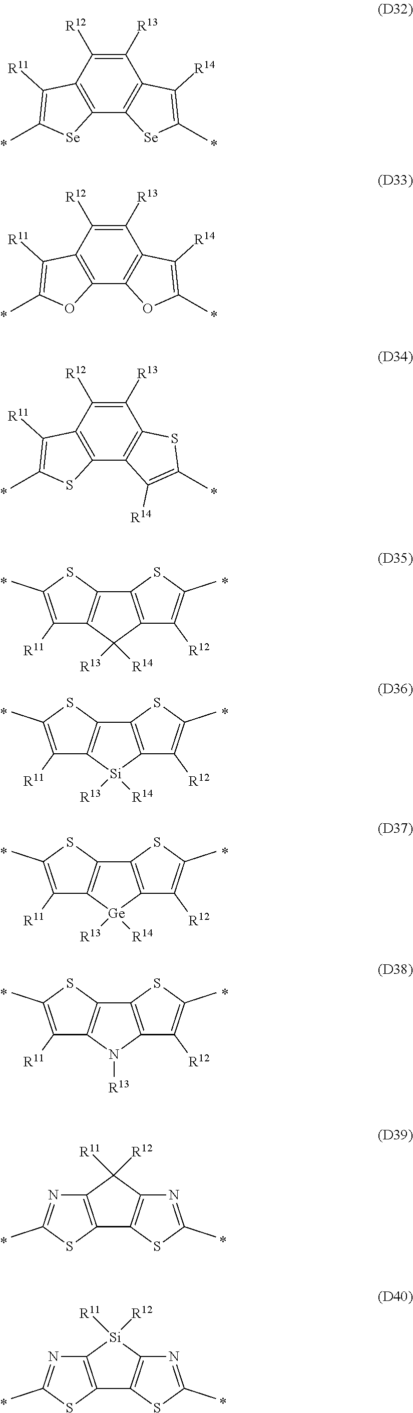

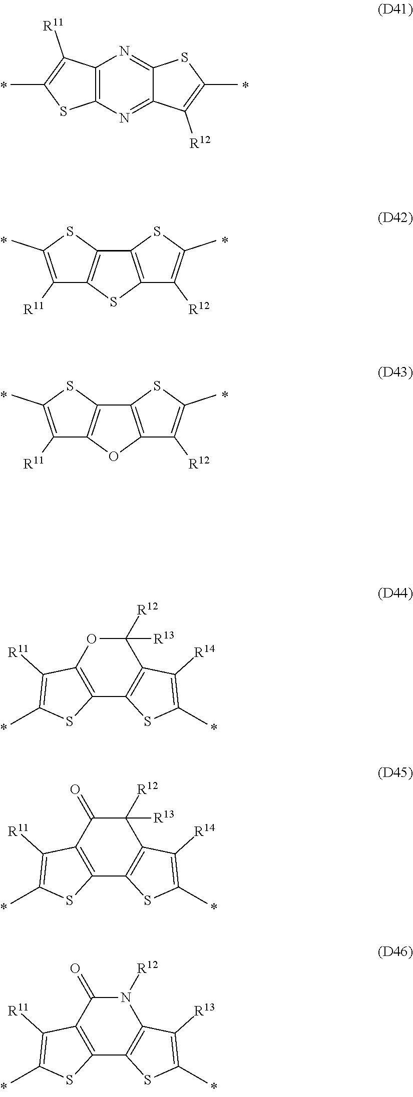

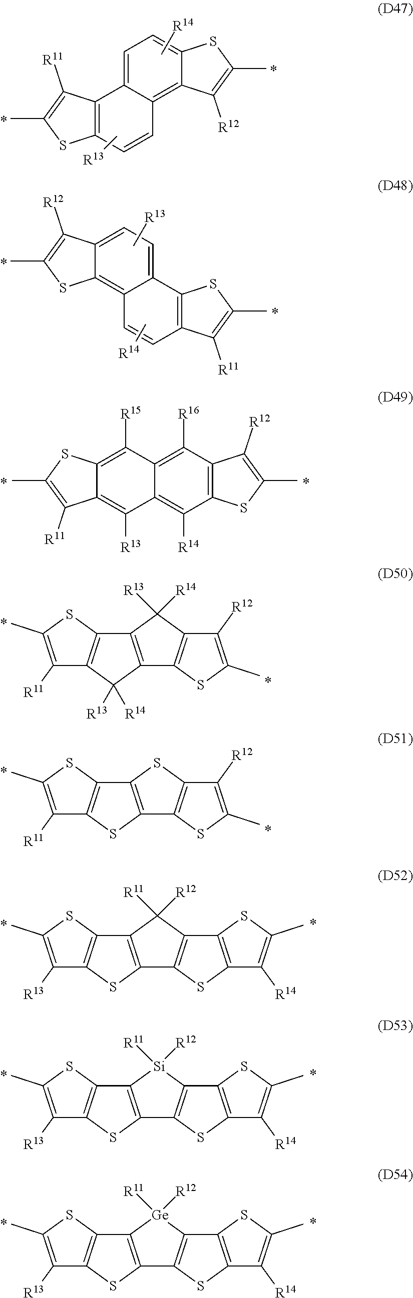

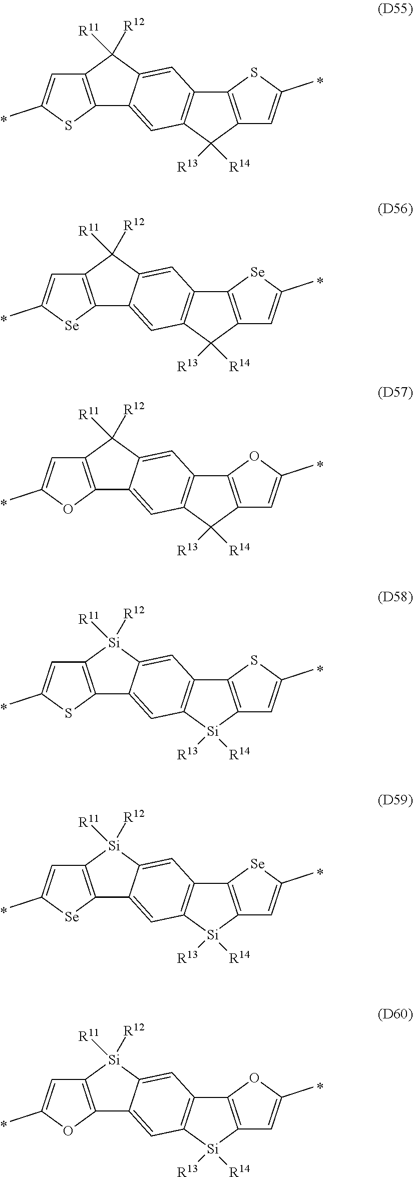

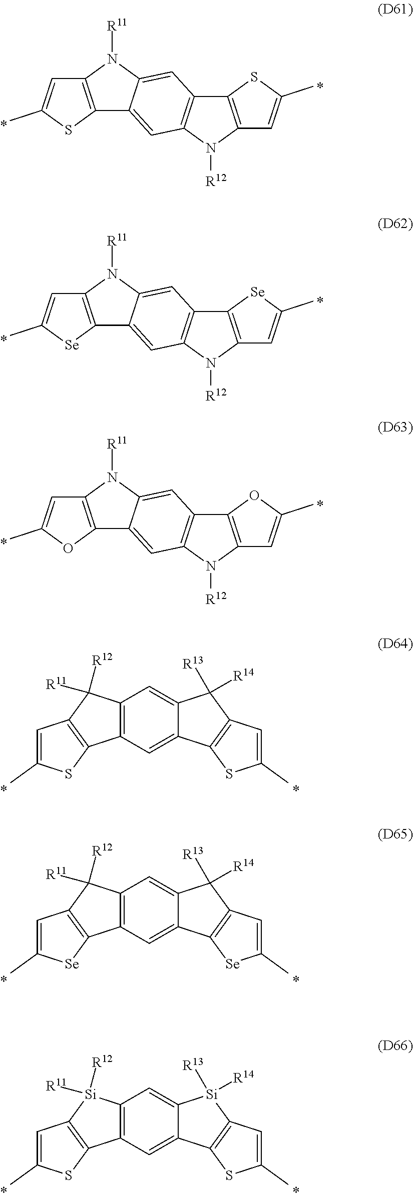

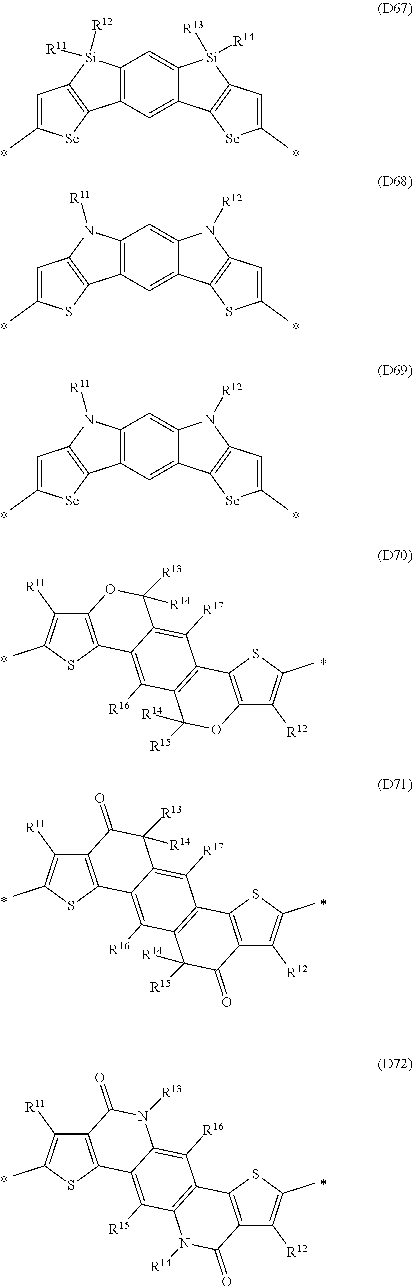

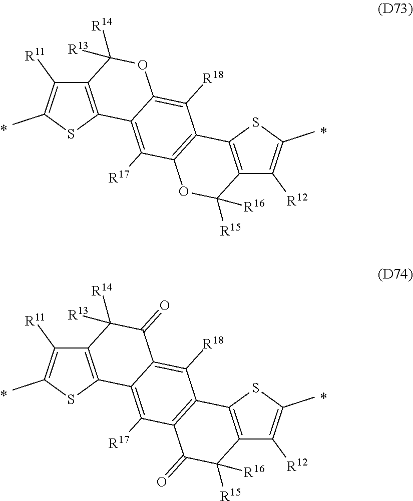

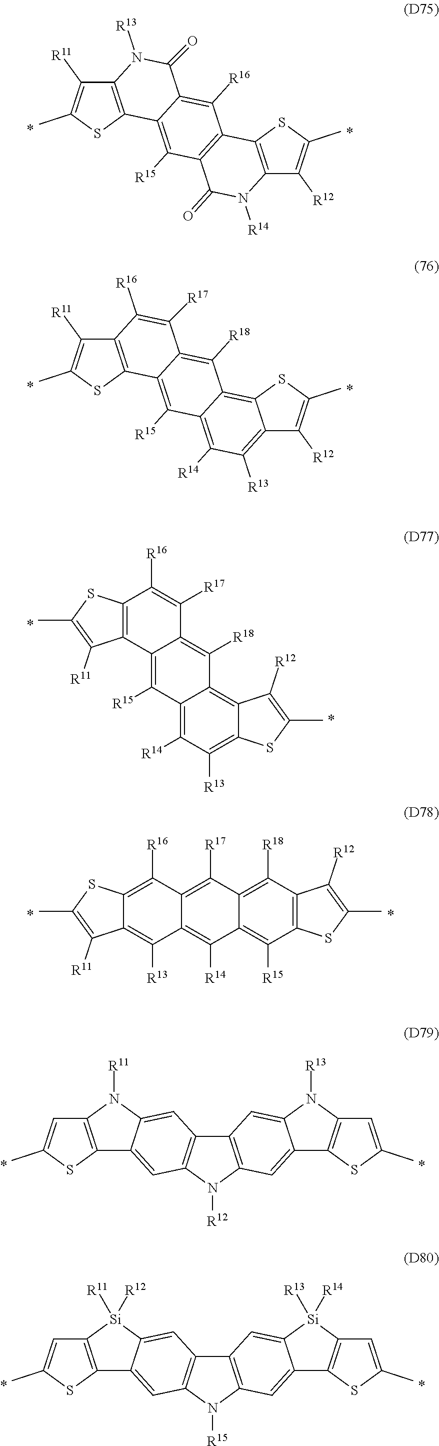

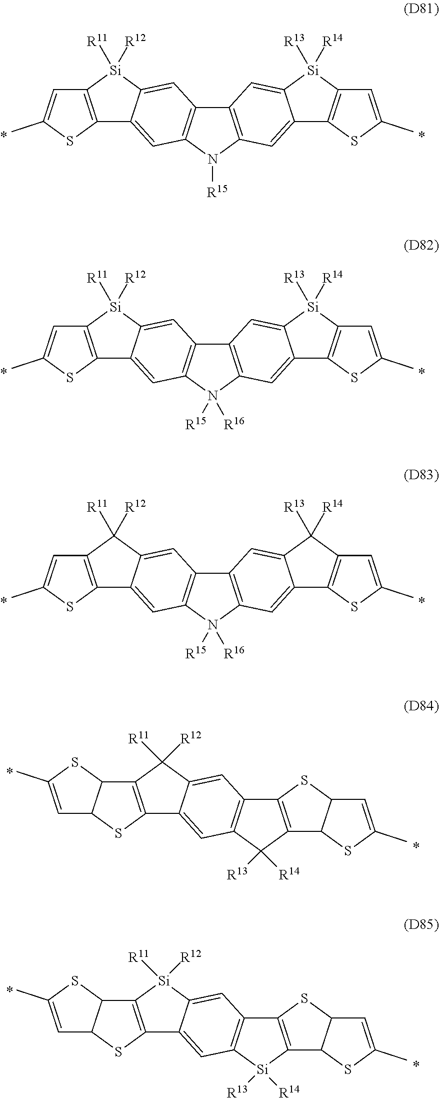

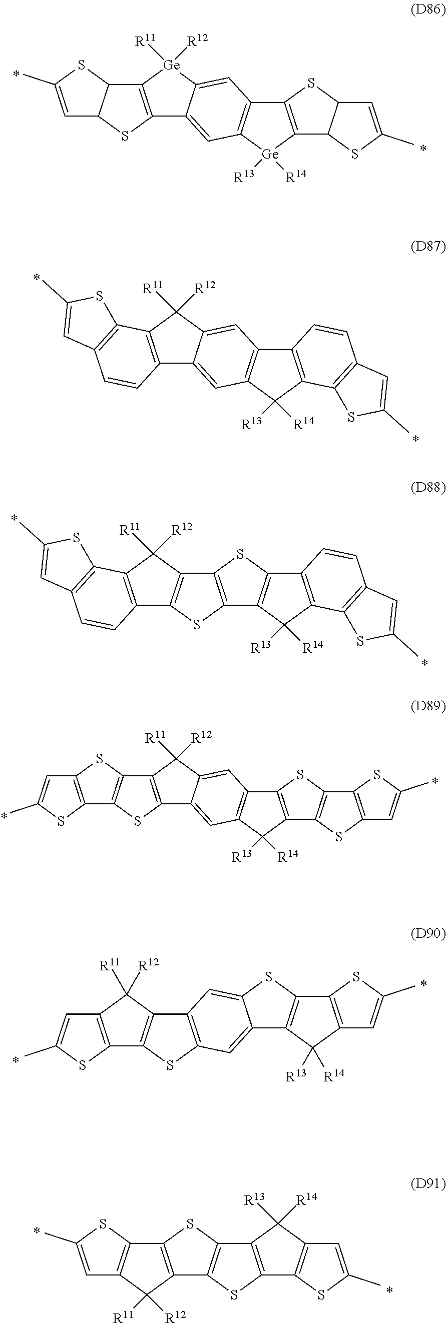

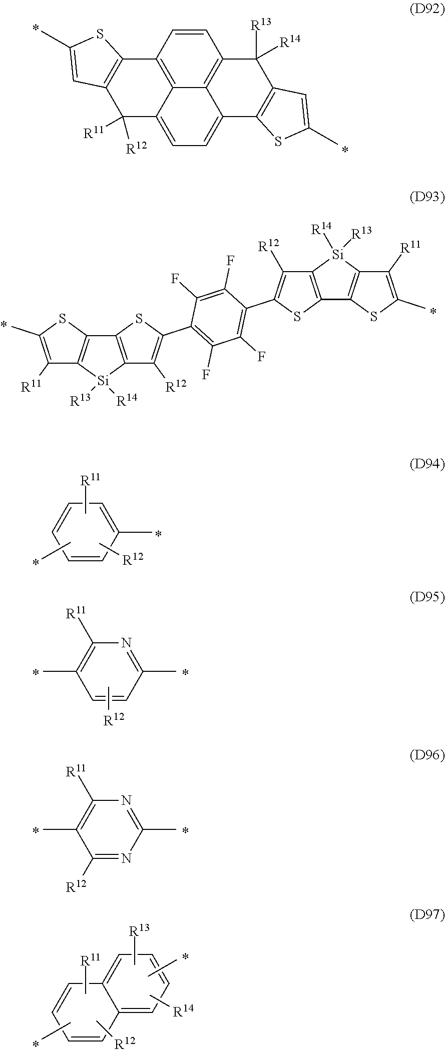

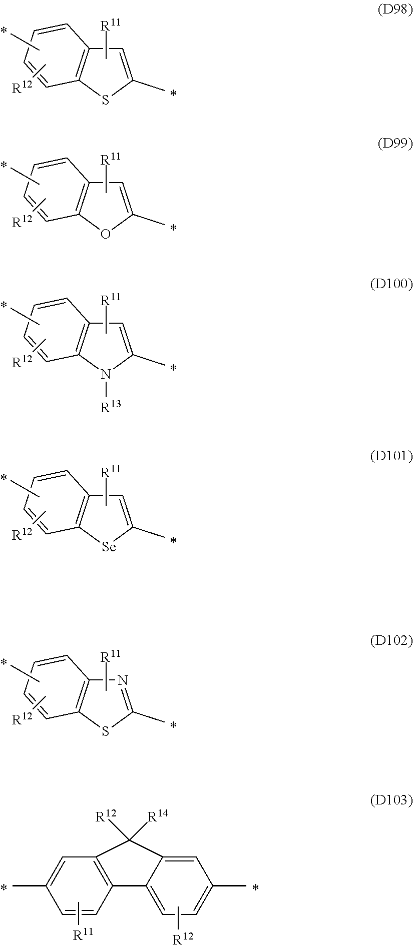

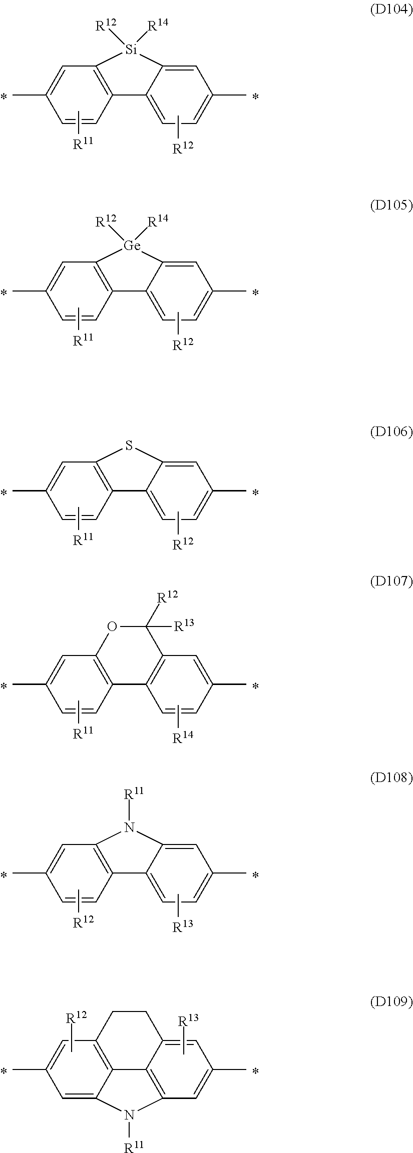

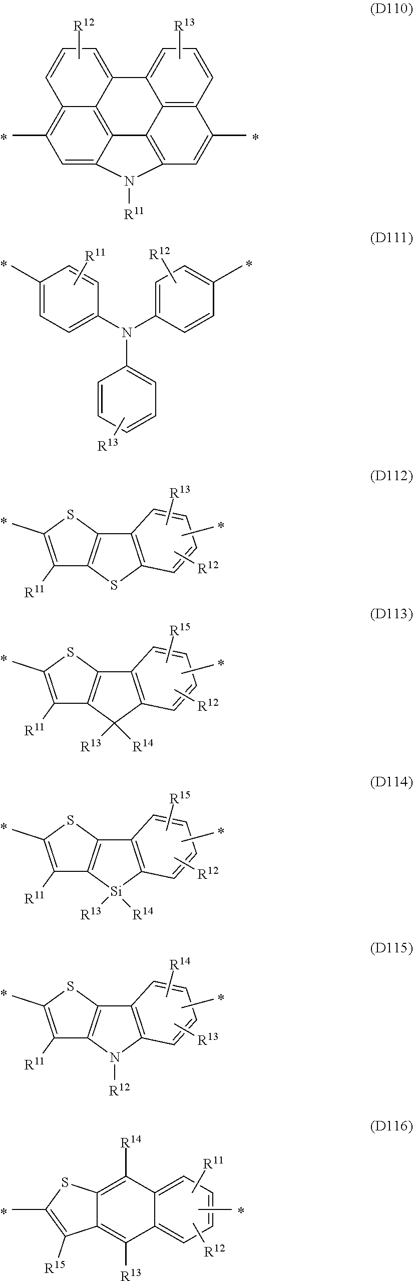

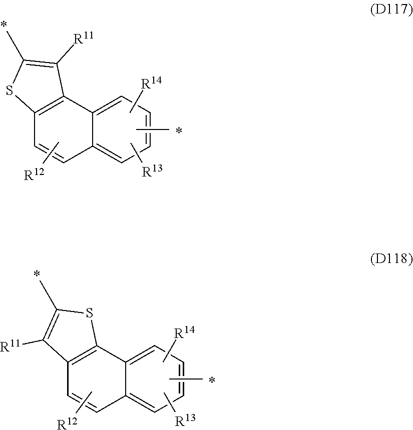

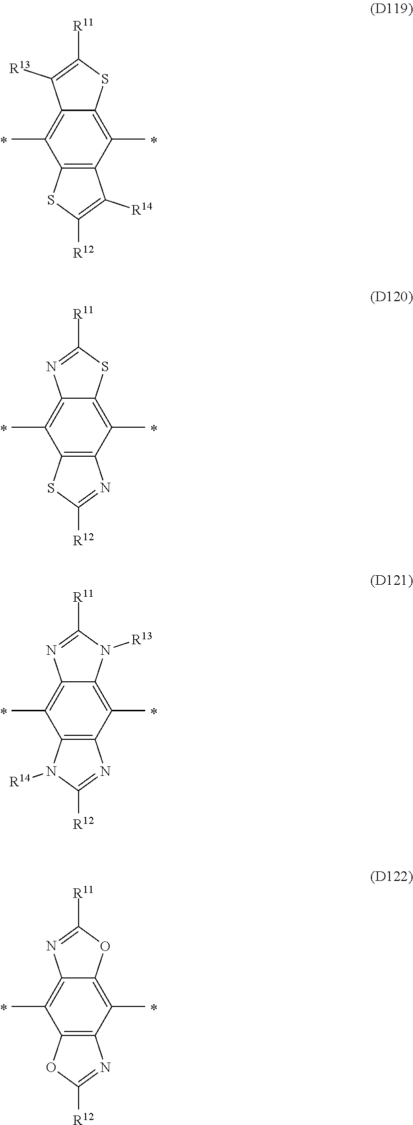

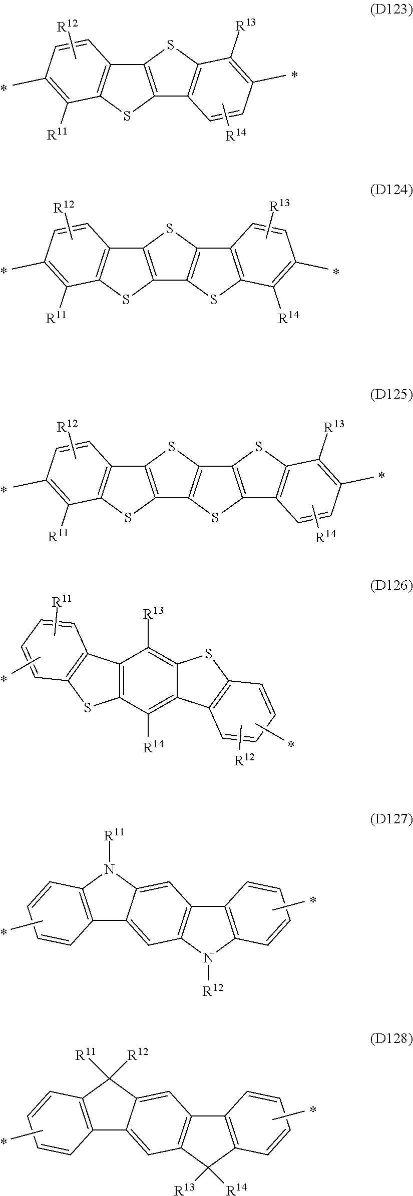

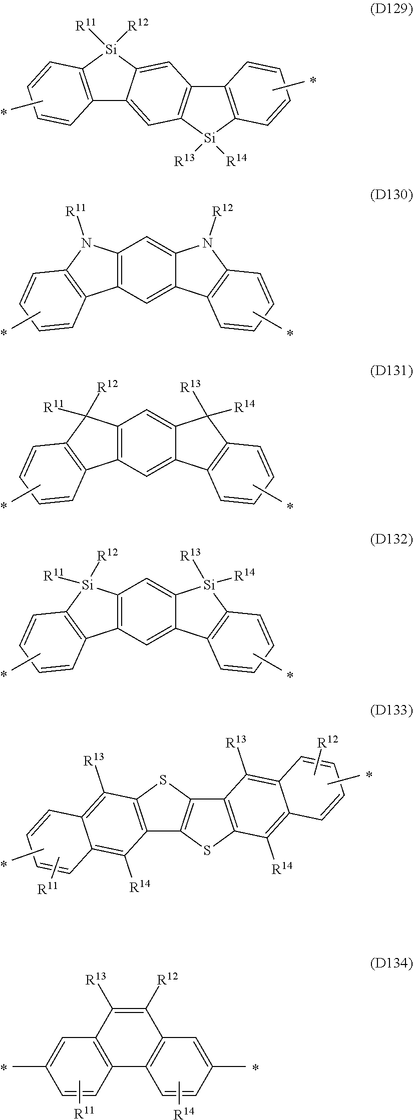

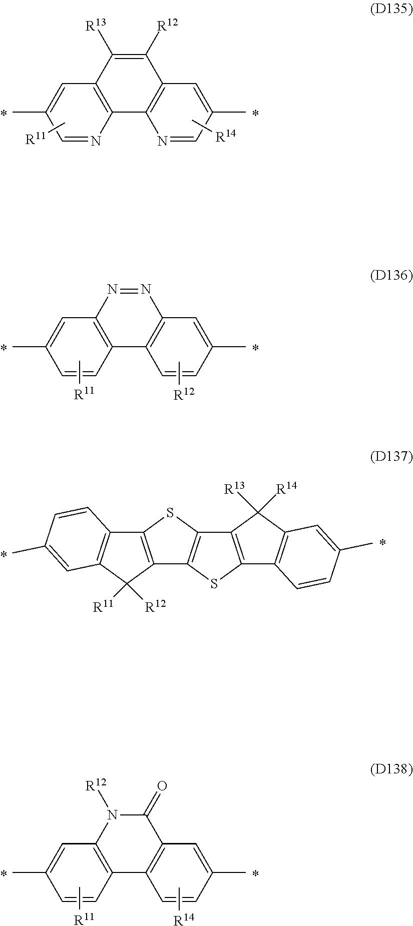

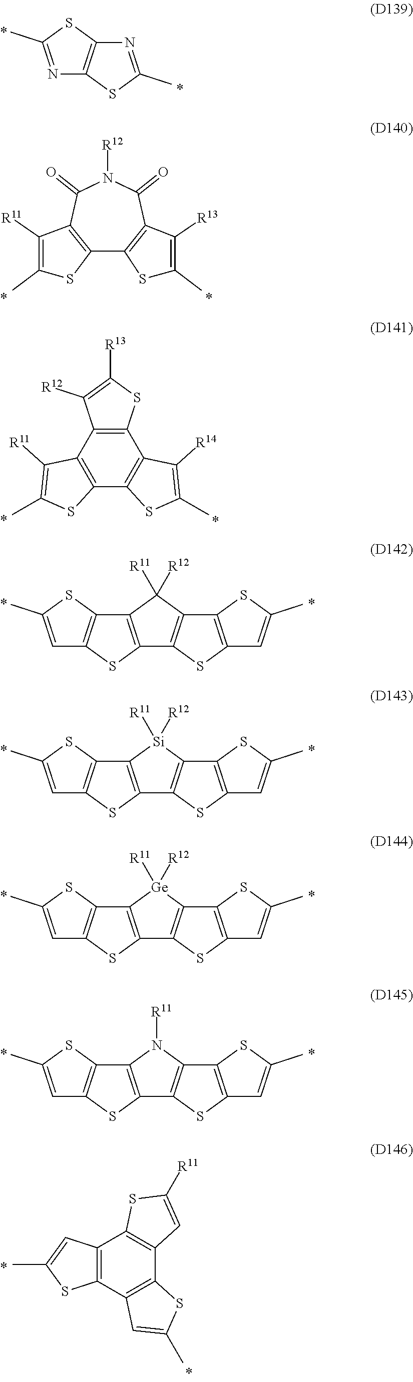

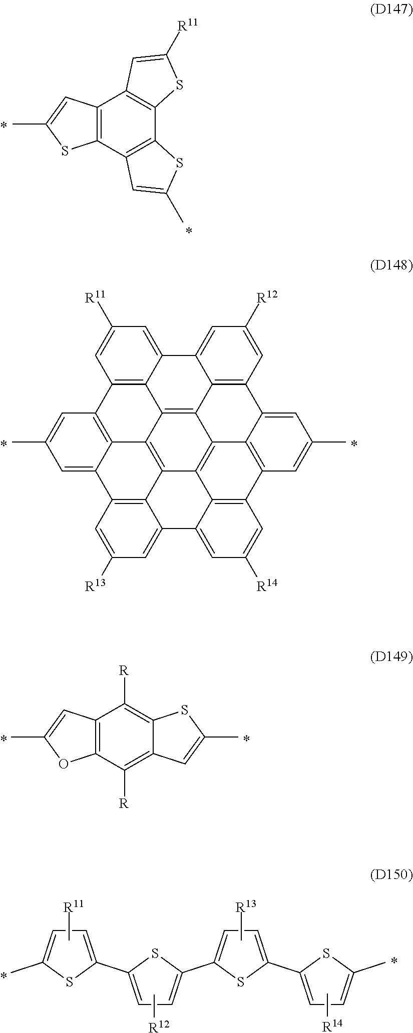

wherein R¹¹, R¹², R¹³, R¹⁴, R¹⁵, R¹⁶, R¹⁷ and R¹⁸ independently of each other denote H or have one of the meanings of L as defined above and below.

Preferred donor units are selected from formulae D1, D7, D10, D11, D19, D22, D29, D30, D35, D36, D37, D44, D55, D84, D87, D88, D89, D93, D94, D106, D111, D139, D140, D141 or D150 wherein preferably at least one of R¹¹, R¹², R¹³ and R¹⁴ is different from H.

Further preferred are repeating units and polymers of formulae II1, II2, III, III1a-III3e, IV and their subformulae wherein one or more of Ar¹, Ar², Ar³ and Ar⁴ denote arylene or heteroarylene, preferably having electron acceptor properties, selected from the group consisting of the following formulae

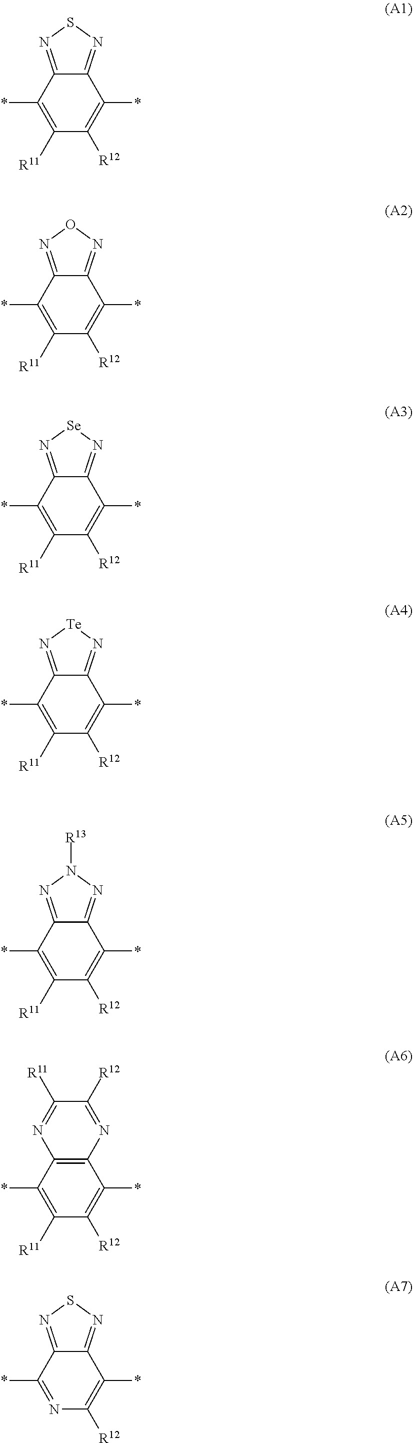

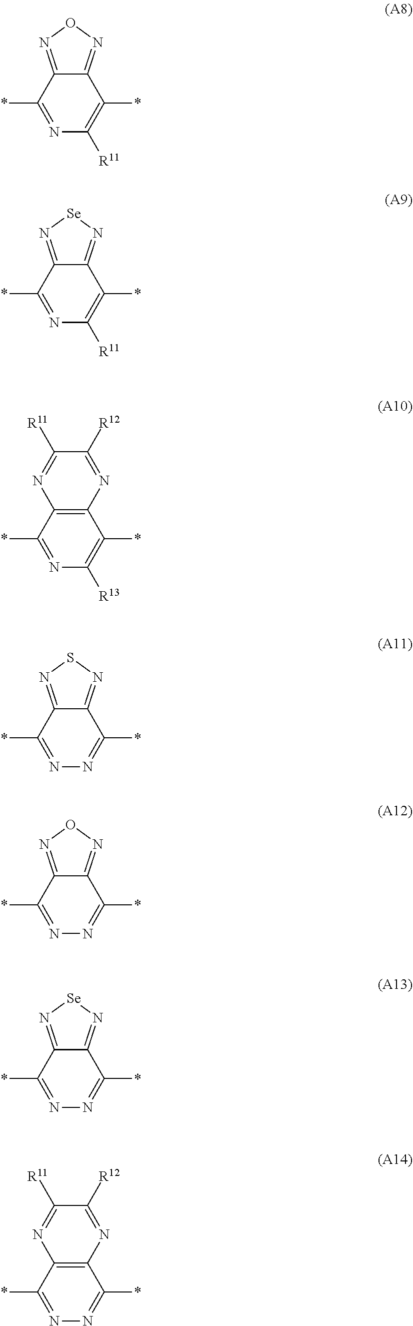

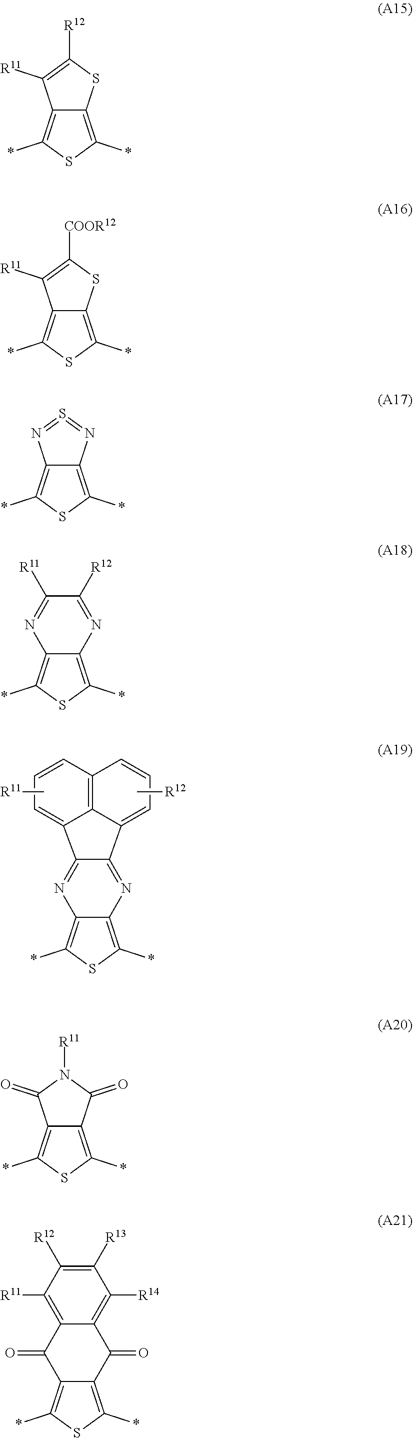

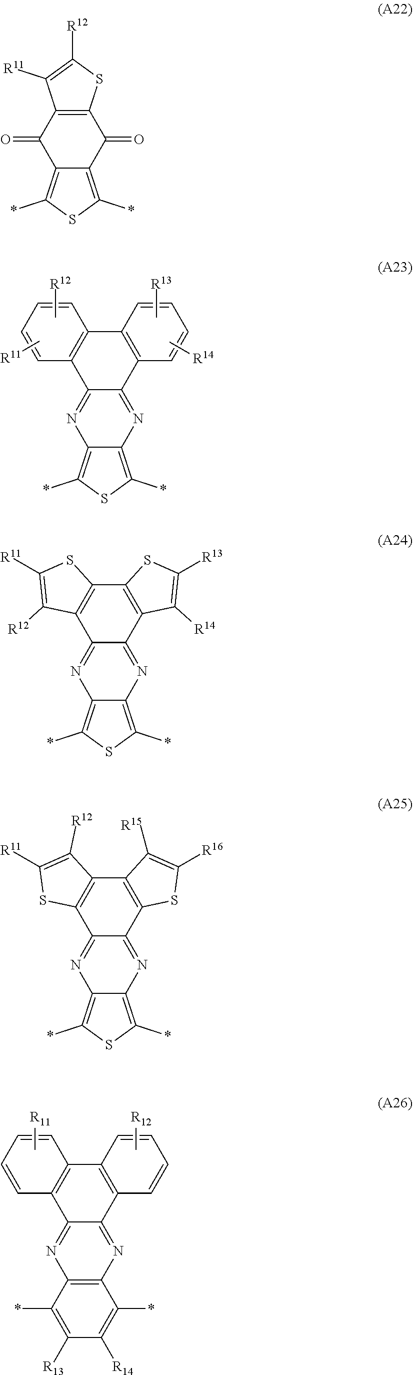

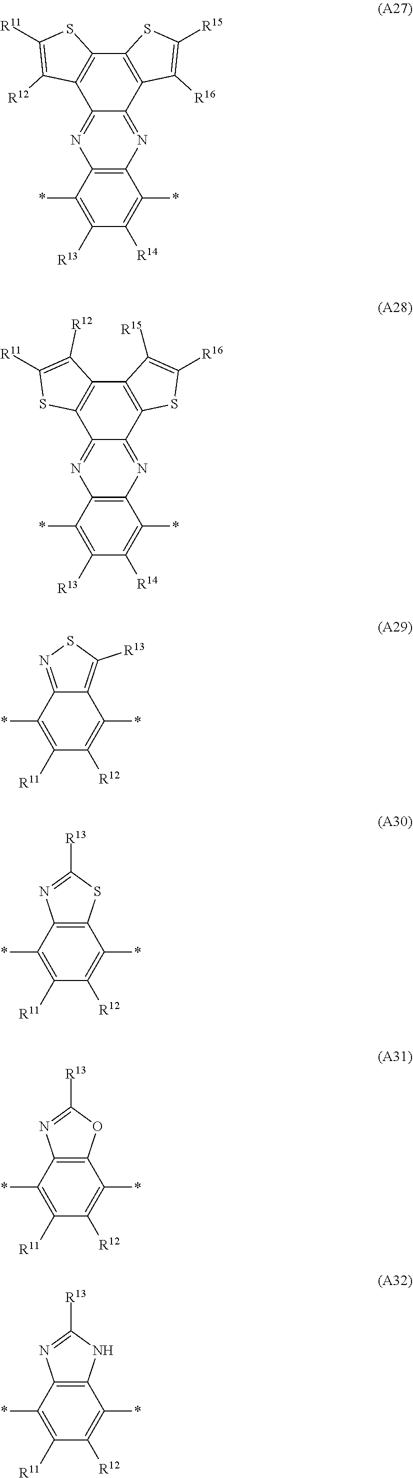

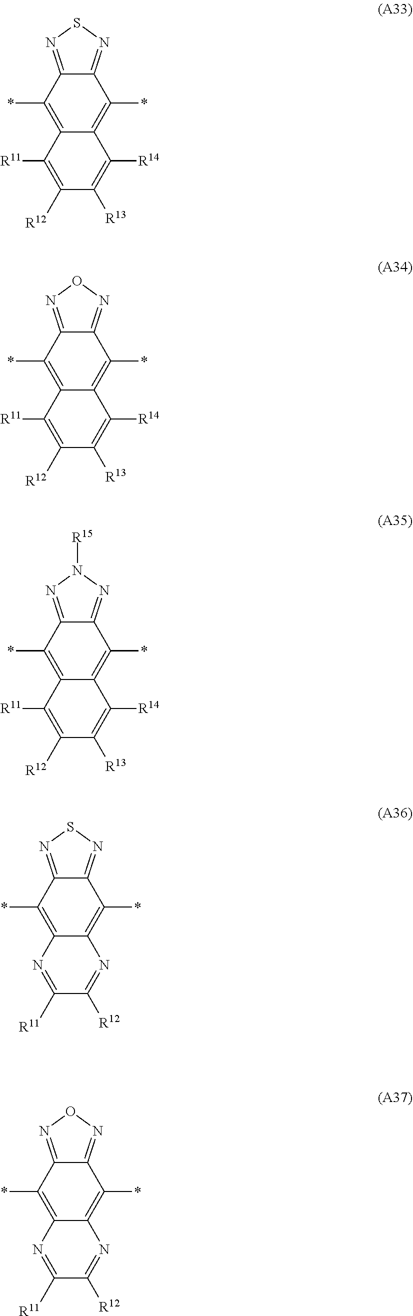

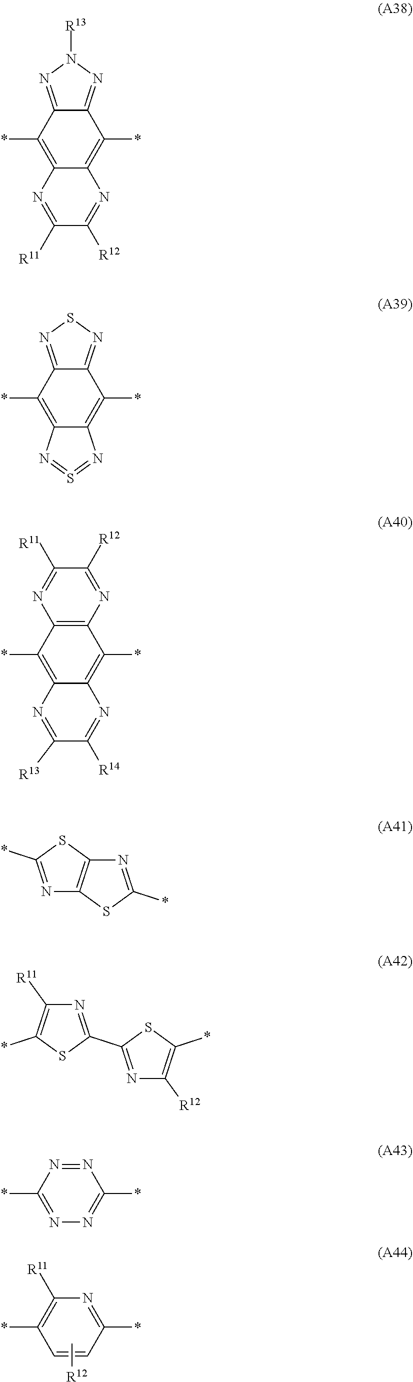

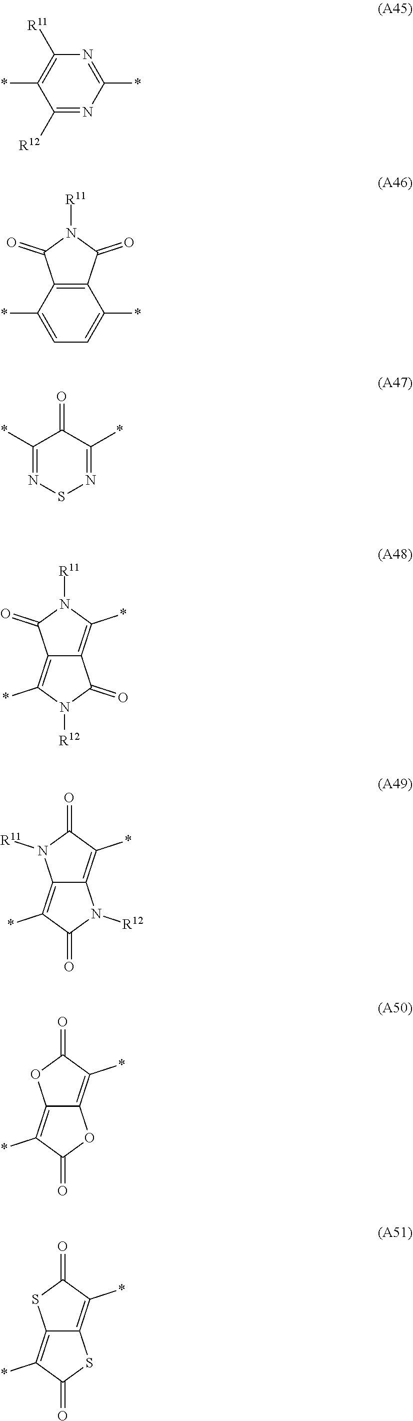

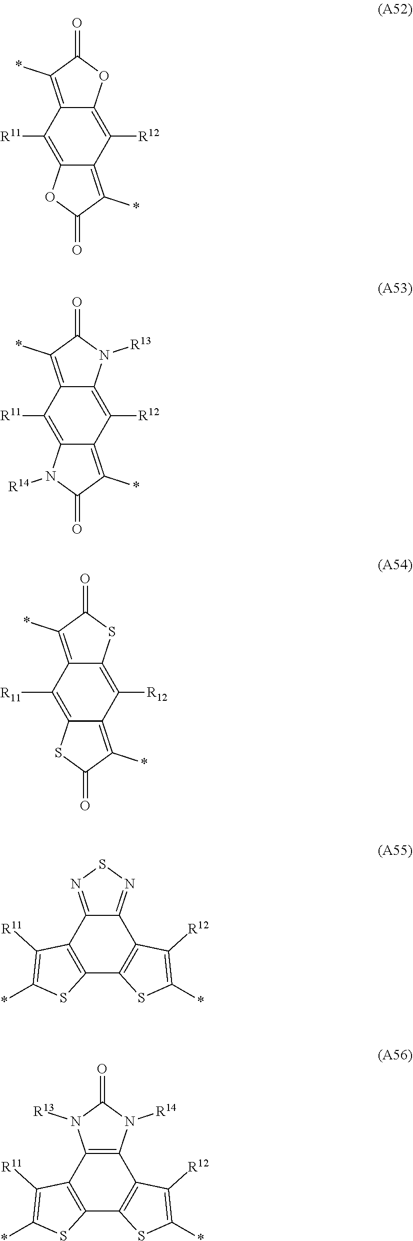

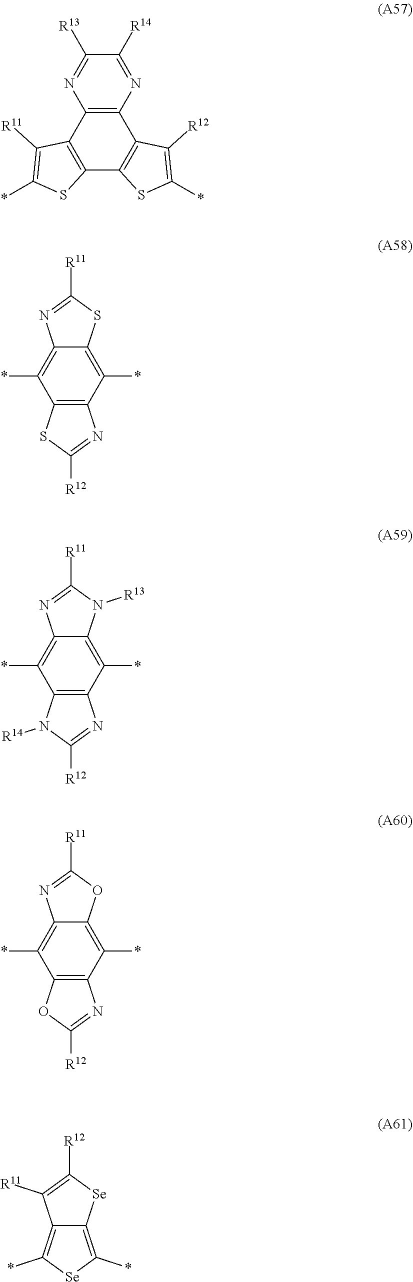

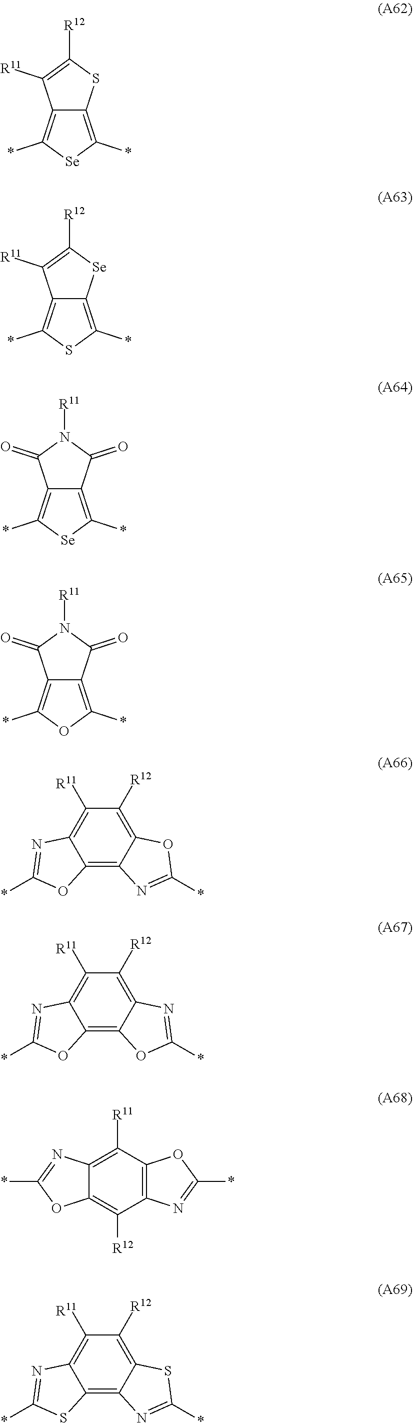

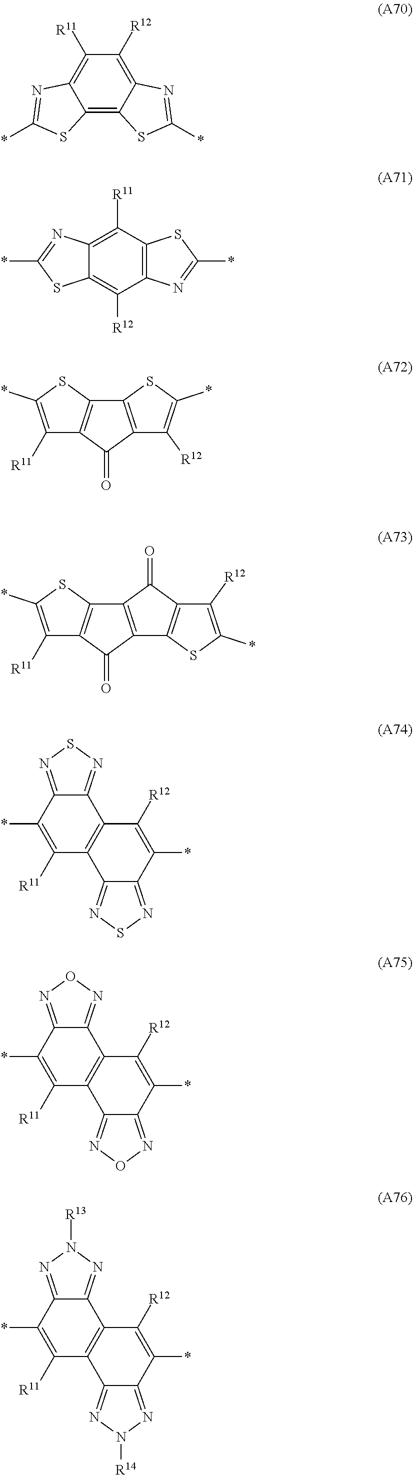

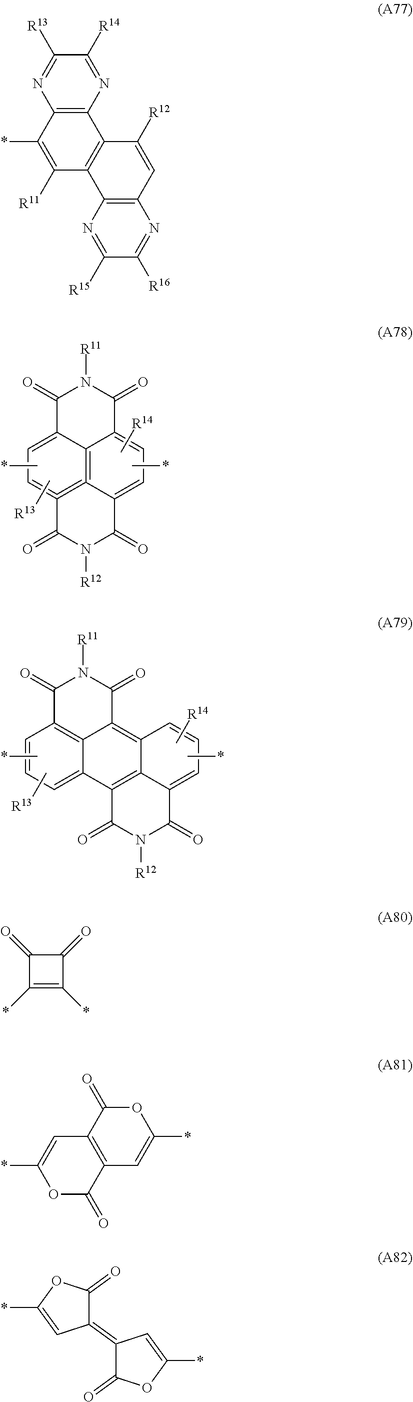

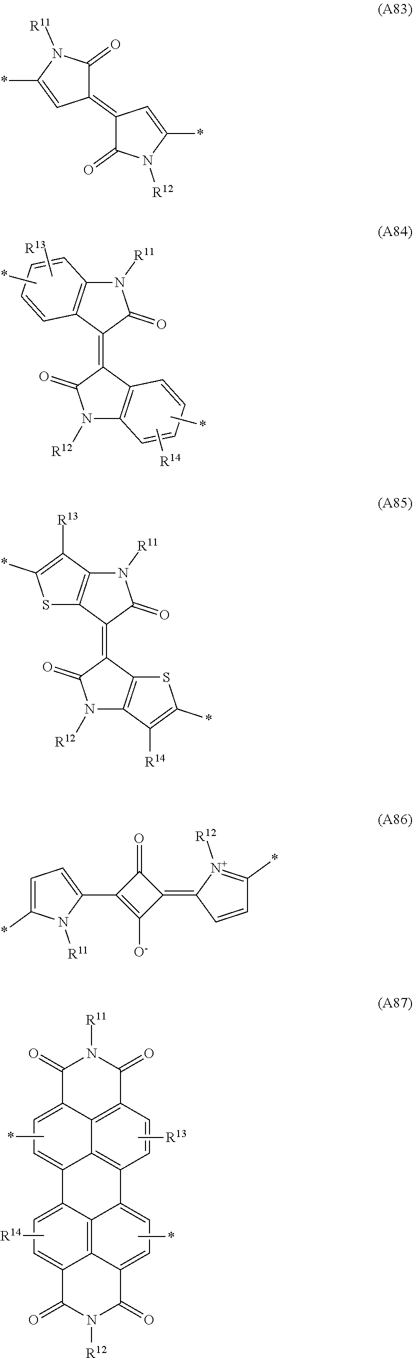

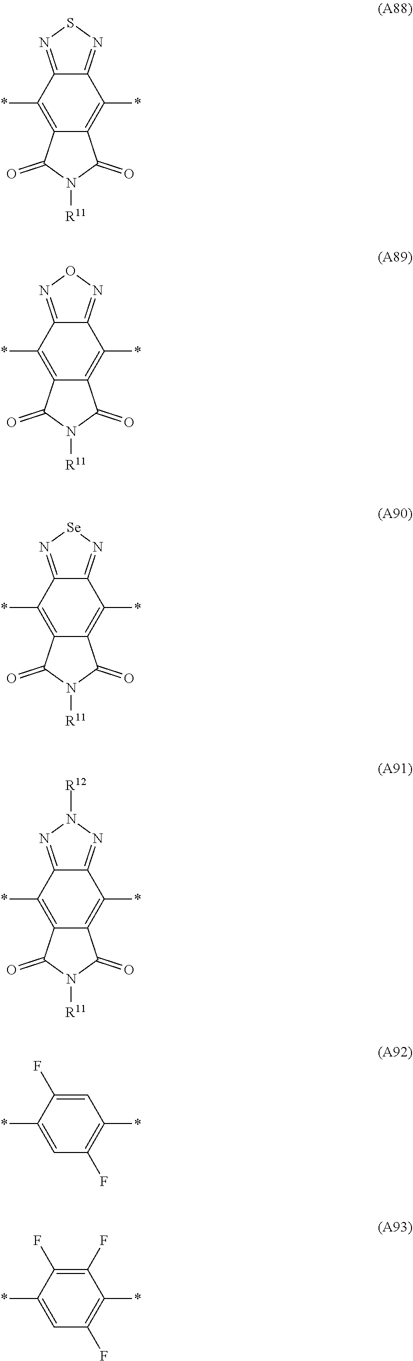

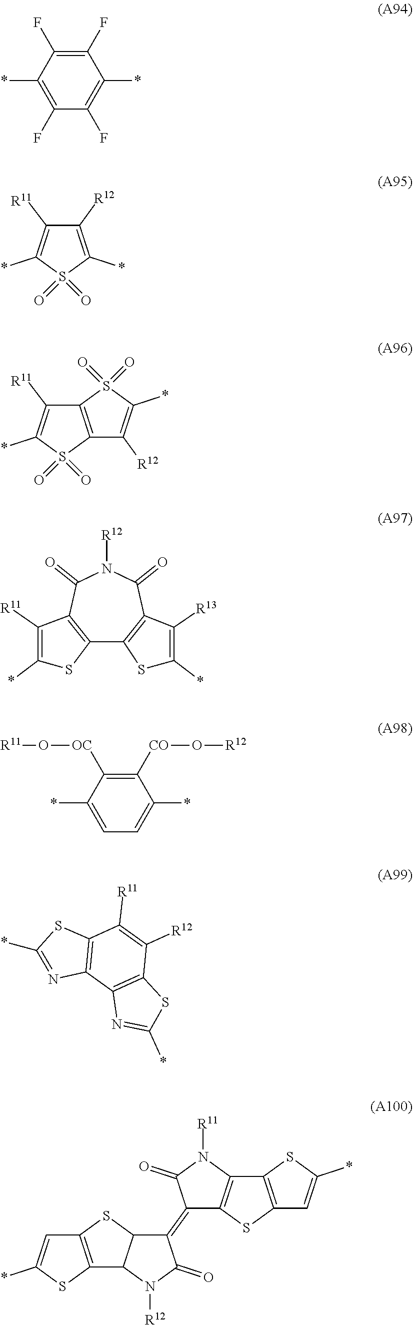

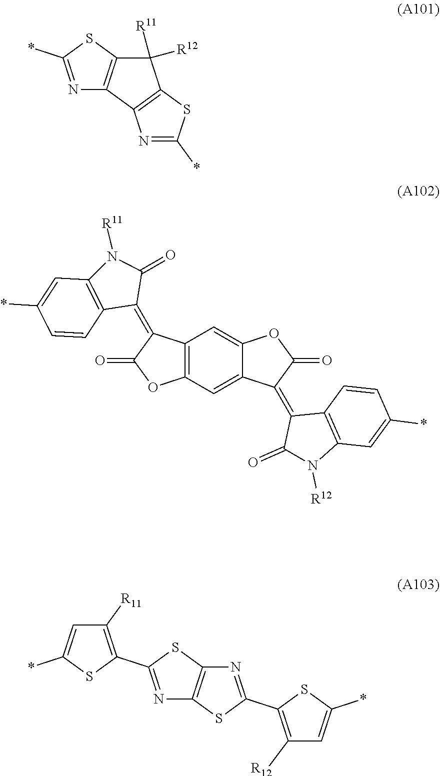

wherein R¹¹, R¹², R¹³, R¹⁴, R¹⁵ and R¹⁶ independently of each other denote H or have one of the meanings of L as defined above and below.

Preferred acceptor units are selected from formulae A1, A6, A7, A15, A16, A20, A36, A74, A84, A88, A92, A98 or A103 wherein preferably at least one of R¹¹, R¹², R¹³ and R¹⁴ is different from H.

Further preferred are repeating units and polymers of formulae II1, II2, III, III1a-III3e, IV and their subformulae wherein one or more of Ar¹, Ar², Ar³ and Ar⁴ denote arylene or heteroarylene selected from the group consisting of the following formulae

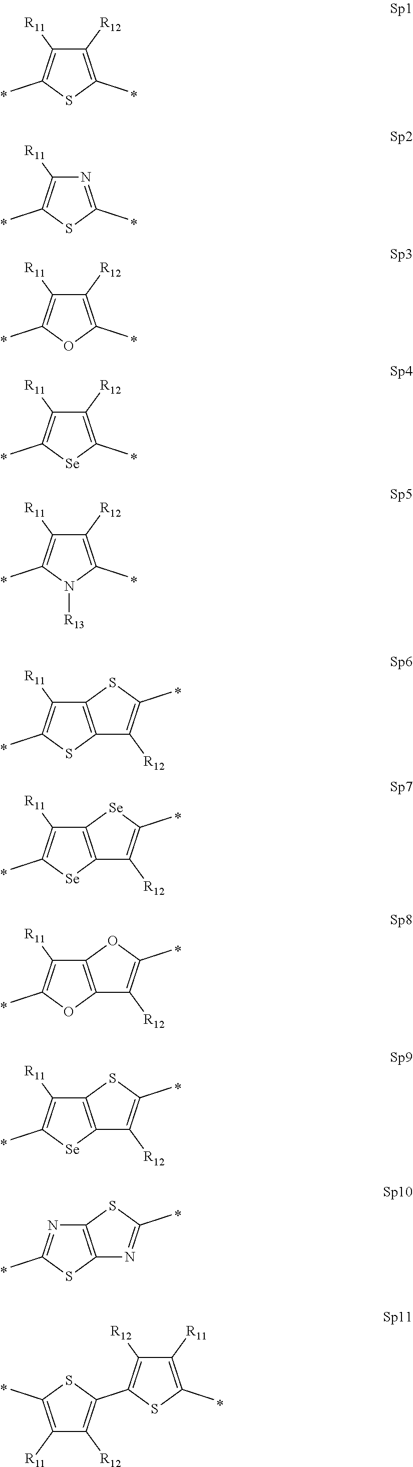

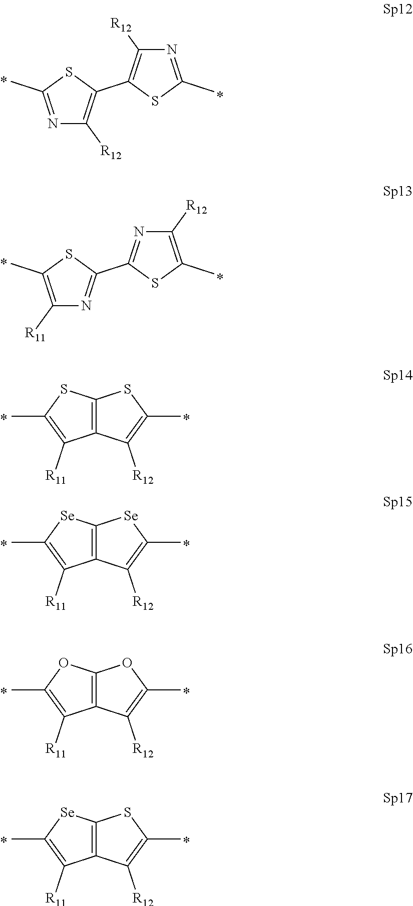

wherein R¹¹ and R¹² independently of each other denote H or have one of the meanings of L as defined above and below.

Very preferred are units selected from formulae Sp1, Sp2, Sp6, Sp10, Sp11, Sp12, Sp13 and Sp14, wherein preferably one of R¹¹ and R¹² is H or both R¹¹ and R¹² are H.

Further preferred are repeating units and polymers of formulae II1, II2, III, III1a-III3e, IV and their subformulae wherein
a) one or more of Ar¹, Ar², Ar³ and Ar⁴ denote arylene or heteroarylene, preferably having electron donor properties, selected from the group consisting of the formulae D1-D145, very preferably of the formulae D1, D7, D10, D11, D19, D22, D29, D30, D35, D36, D37, D44, D55, D84, D87, D88, D89, D93, D94, D106, D111, D139, D140, D141 and D150, and/or
b) one or more of Ar¹, Ar², Ar³ and Ar⁴ denote arylene or heteroarylene, preferably having electron accpetor properties, selected from the group consisting of the formulae A1-A98, very preferably of the formulae A1, A6, A7, A15, A16, A20, A36, A74, A84, A88, A92, A98 and A103, and
c) one or more of Ar¹, Ar², Ar³ and Ar⁴ denote arylene or heteroarylene selected from the group consisting of the formulae Sp1-Sp17, very preferably of the formulae Sp1, Sp2, Sp6, Sp10, Sp11, Sp12, Sp13 and Sp14.

Another preferred embodiment of the present invention relates to a small molecule or oligomer of formula VI

R^{T1}—(Ar¹)ₑ—(Ar²)_{f}—[(Ar³)_{g}—(Ar⁴)ₕ—U—(Ar⁵)ᵢ—(Ar⁶)ₖ]ₒ—(Ar⁷)ₗ—(Ar⁸)ₘ—R^{T2} VI

wherein the individual radicals, independently of each other and on each occurrence identically or differently, have the following meanings
U a unit of formula I or its subformulae,
Ar¹⁻⁸ arylene or heteroarylene that has from 5 to 20 ring atoms, is mono- or polycyclic, optionally contains fused rings, and is unsubstituted or substituted by one or more identical or different groups L as defined in formula I, or —CY¹═CY²— or
Y¹, Y² H, F, Cl or CN,
R^{T1}, R^{T2} a carbyl or hydrocarbyl group with 1 to 30 C atoms that is optionally substituted by one or more groups L and optionally comprises one or more hetero atoms,
e-m 0 or 1, preferably with at least one of e-m being 1,
o 1, 2 or 3.

Preferred groups R^{T1} and R^{T2} in formula I are selected from H, F, Cl, Br, —NO₂, —CN, —CF₃, R*, —CF₂—R*, —O—R*, —S—R*, —SO₂—R*, —SO₃—R*, —C(═O)—H, —C(═O)—R*, —C(═S)—R*, —C(═O)—CF₂—R*, —C(═O)—OR*, —C(═S)—OR*, —O—C(═O)—R*, —O—C(═S)—R*, —C(═O)—SR*, —S—C(═O)—R*, —C(═O)NR*R**, —NR*—C(═O)—R*, —NHR*, —NR*R**, —CR*═CR*R**, —C═C—R*, —C═C—SiR*R**R***, —SiR*R**R***, —CH═CH(CN), —CH═C(CN)₂, —C(CN)═C(CN)₂, —CH═C(CN)(R^{a}), CH═C(CN)—C(═O)—OR*, —CH═C(CO—OR*)₂, —CH═C(CO—NR*R**)₂, and the group consisting of the following formulae

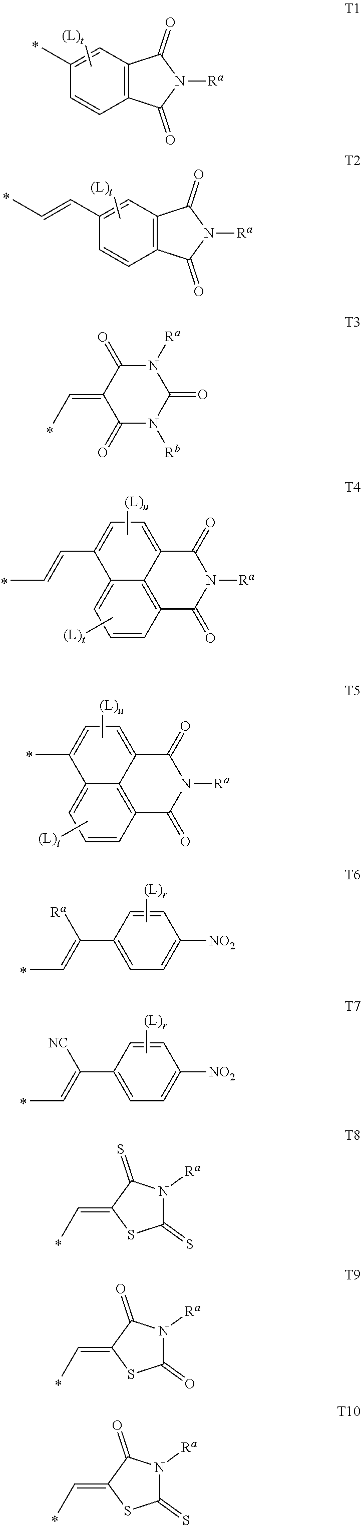

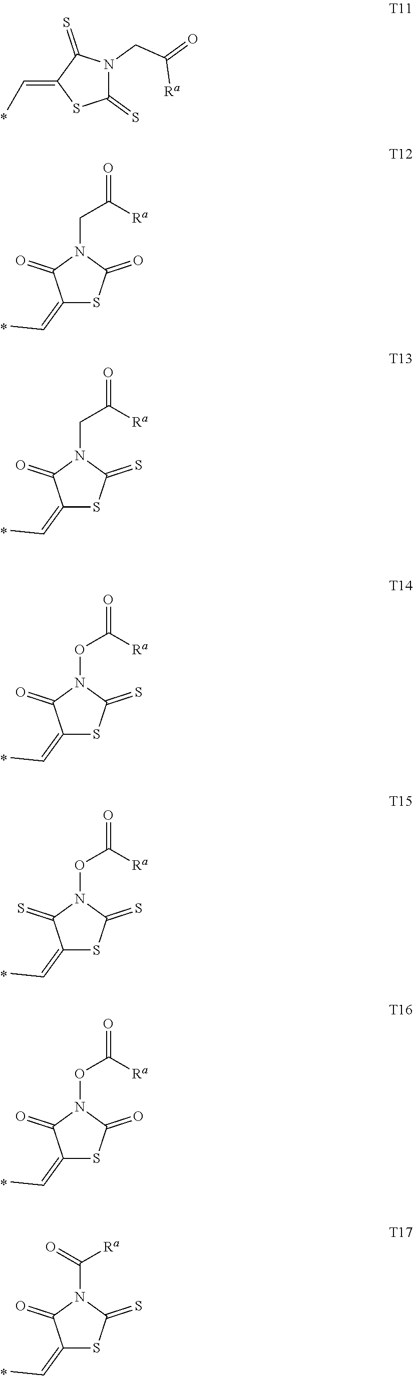

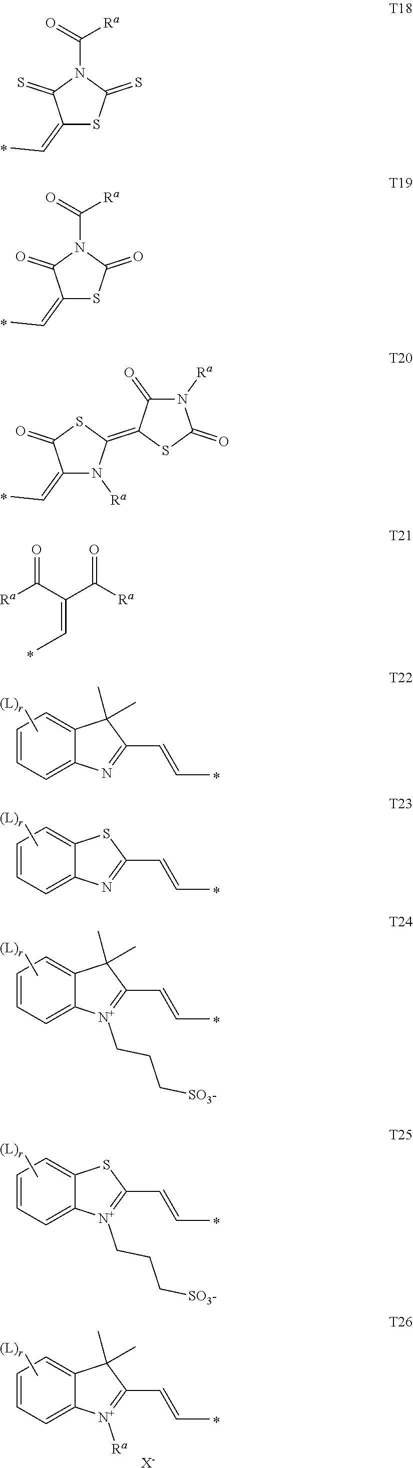

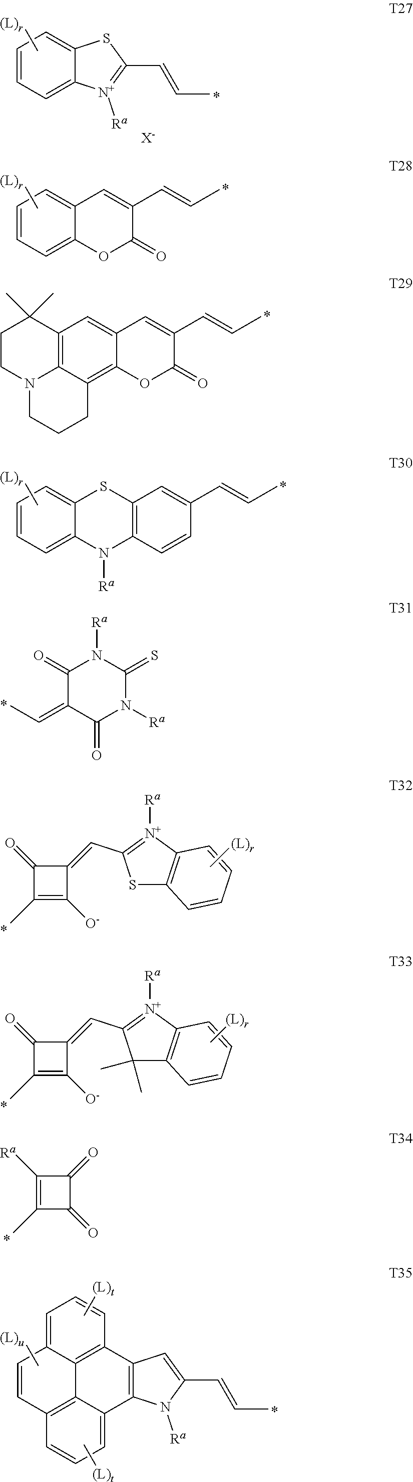

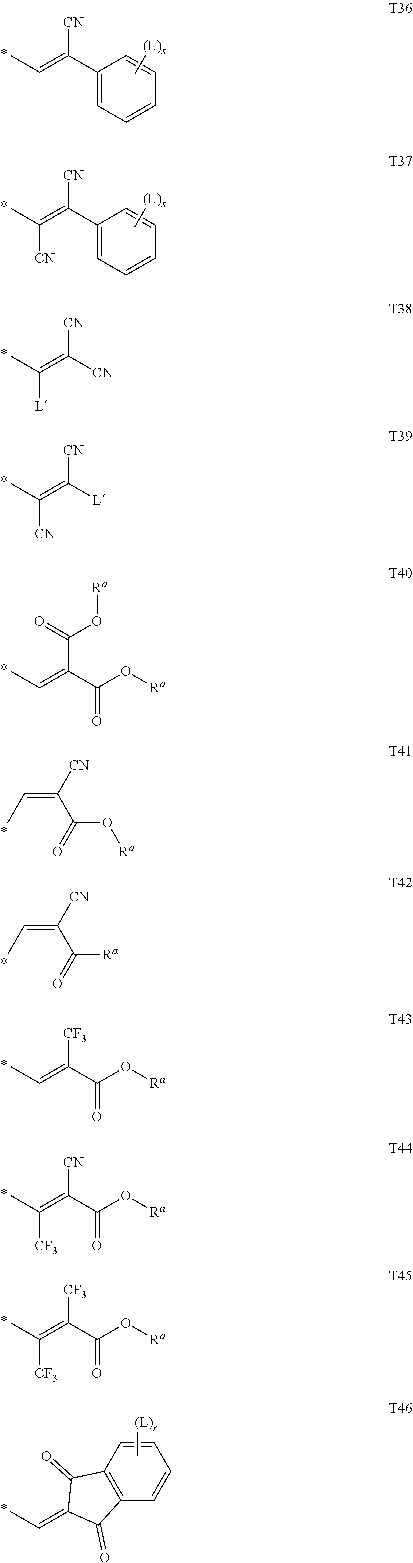

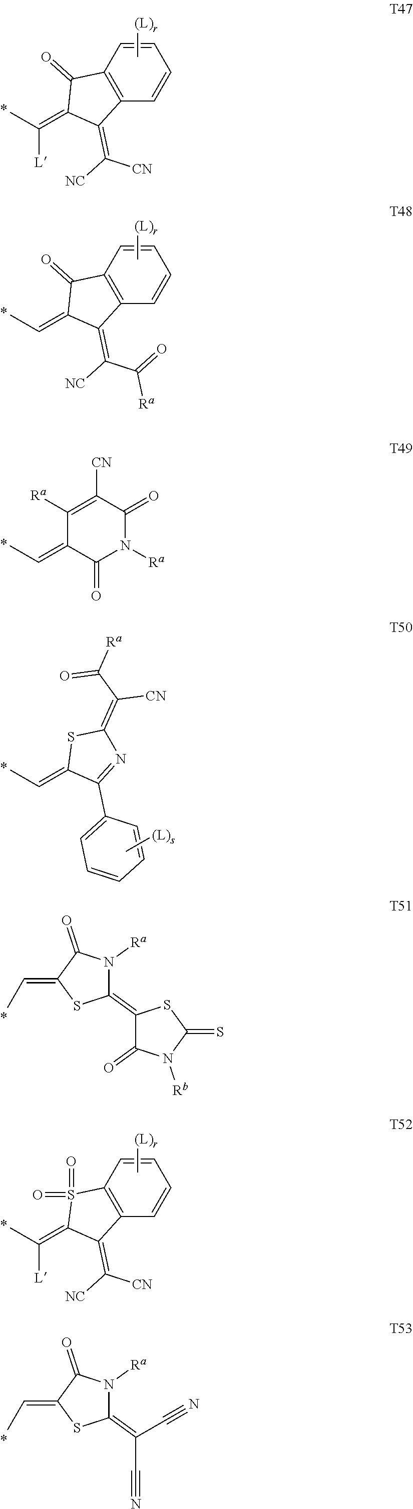

wherein the individual radicals, independently of each other and on each occurrence identically or differently, have the following meanings
R^{a}, R^{b} aryl or heteroaryl, each having from 4 to 30 ring atoms, optionally containing fused rings and being unsubstituted or substituted with one or more groups L, or one of the meanings given for L,
R*, R**, R*** alkyl with 1 to 20 C atoms which is straight-chain, branched or cyclic, and is unsubstituted, or substituted with one or more F or Cl atoms or CN groups, or perfluorinated, and in which one or more C atoms are optionally replaced by —O—, —S—, —C(═O)—, —C(═S)—, —SiR⁰R⁰⁰—, —NR⁰R⁰⁰—, —CHR⁰═CR⁰⁰— or —C≡C— such that O- and/or S-atoms are not directly linked to each other,

L F, Cl, —NO₂, —CN, —NC, —NCO, —NCS, —OCN, —SCN, R⁰, OR⁰, SR⁰, —C(═O)X⁰, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO₂R⁰, —OH, —NO₂, —CF₃, —SF₅, or optionally substituted silyl, or carbyl or hydrocarbyl with 1 to 30, preferably 1 to 20, C atoms that is optionally substituted and optionally comprises one or more hetero atoms, preferably F, —CN, R⁰, —OR⁰, —SR⁰, —C(═O)—R⁰, —C(═O)—OR⁰, —O—C(═O)—R⁰, —O—C(═O)—OR⁰, —C(═O)—NHR⁰, —C(═O)—NR⁰R⁰⁰,
L′ H or one of the meanings of L,
R⁰, R⁰⁰H or straight-chain or branched alkyl with 1 to 20, preferably 1 to 12 C atoms that is optionally fluorinated,
Y¹, Y² H, F, Cl or CN,
X⁰ halogen, preferably F or Cl,
r 0, 1, 2, 3 or 4,
s 0, 1, 2, 3, 4 or 5,
t 0, 1, 2 or 3,
u 0, 1 or 2.

Preferred compounds of formula VI are those wherein one or both, preferably both, of R^{T1} and R^{T2} denote an electron withdrawing group.

Preferred electron withdrawing groups R^{T1} and R^{T2} are selected from —CN, —C(═O)—OR*, —C(═S)—OR*, —CH═CH(CN), —CH═C(CN)₂, —C(CN)═C(CN)₂, —CH═C(CN)(R^{a}), CH═C(CN)—C(═O)—OR*, —CH═C(CO—OR*)₂, and formulae T1-T53.

Very preferred groups R^{T1} and R^{T2} are selected from the following formulae

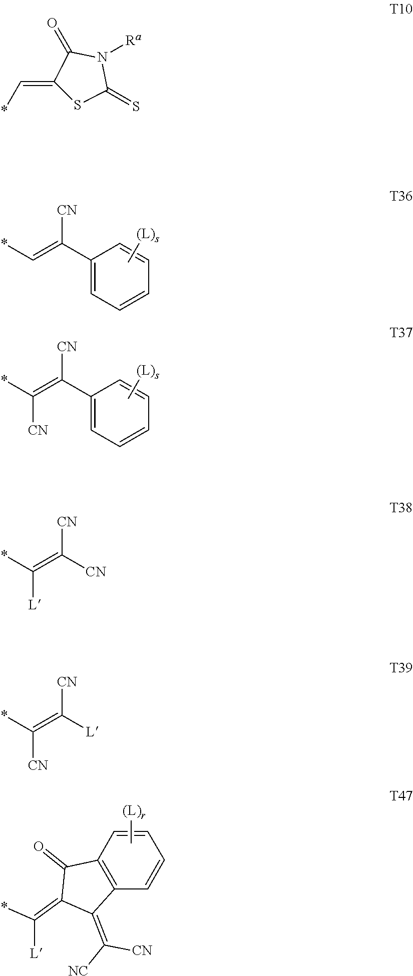

wherein L, r and s have the meanings given above and below, and L′ is H or has one of the meanings given for L. Preferably in these formulae L′ is H. Further preferably in these formulae r is 0.

The above formulae T1-T53 are meant to also include their respective E- or Z-stereoisomer with respect to the C═C bond in α-position to the adjacent group Ar^{1.8}, thus for example the group

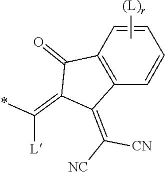

may also denote

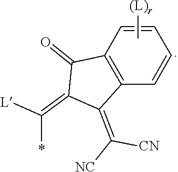

Preferred compounds of formula VI are selected from the following subformulae

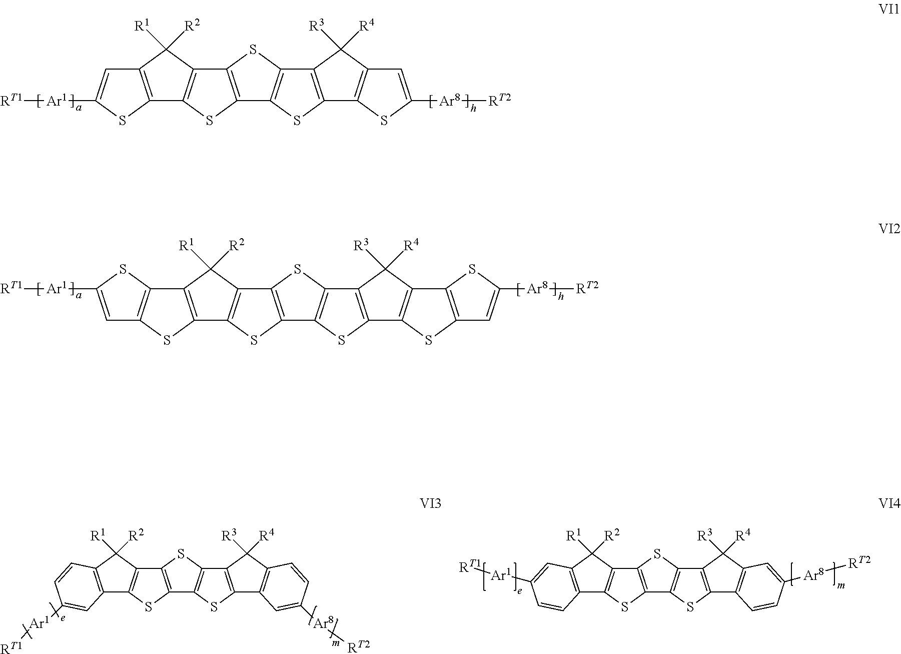

wherein R¹⁻⁴, R^{T1}, R^{T2}, Ar¹, Ar⁸, e and m have the meanings given above.

Further preferred are compounds of formula VI wherein Ar¹⁻⁸ are selected from the following groups
a) the group consisting of the formulae D1-D145, very preferably of the formulae D1, D7, D10, D11, D19, D22, D29, D30, D35, D36, D37, D44, D55, D84, D87, D88, D89, D93, D94, D106, D111, D139, D140, D141 and D150,
b) the group consisting of the formulae A1-A98, very preferably of the formulae A1, A6, A7, A15, A16, A20, A36, A74, A84, A88, A92, A98 and A103,
c) the group consisting of the formulae Sp1-Sp17, very preferably of the formulae Sp1, Sp2, Sp6, Sp10, Sp11, Sp12, Sp13 and Sp14.

Further preferred compounds of formula VI are selected from the following preferred embodiments, including any combination thereof:
m is 1,
a and h are 0, 1 or 2, and c-f are 0,
Ar¹⁻⁸ are selected from formulae Sp1, Sp2, Sp6, Sp10, Sp11, Sp12, Sp13 and Sp14,
R^{T1} and R^{T2} are selected from formulae T10, T36, T37, T38, T39 and T47,
R^{T1} and R^{T2} are selected from formula T47.

Further preferred compounds of formula VI are those wherein m is 1, a and h are 0 or 1, c-f are 0, Ar¹⁻⁸ are selected from formulae Sp1, Sp2, Sp6, Sp10, Sp11, Sp12, Sp13 and Sp14, and R^{T1} and R^{T2} are selected from formulae R1-R5, very preferably from formula R5.

Further preferred compounds of formula VI are selected from the following subformulae

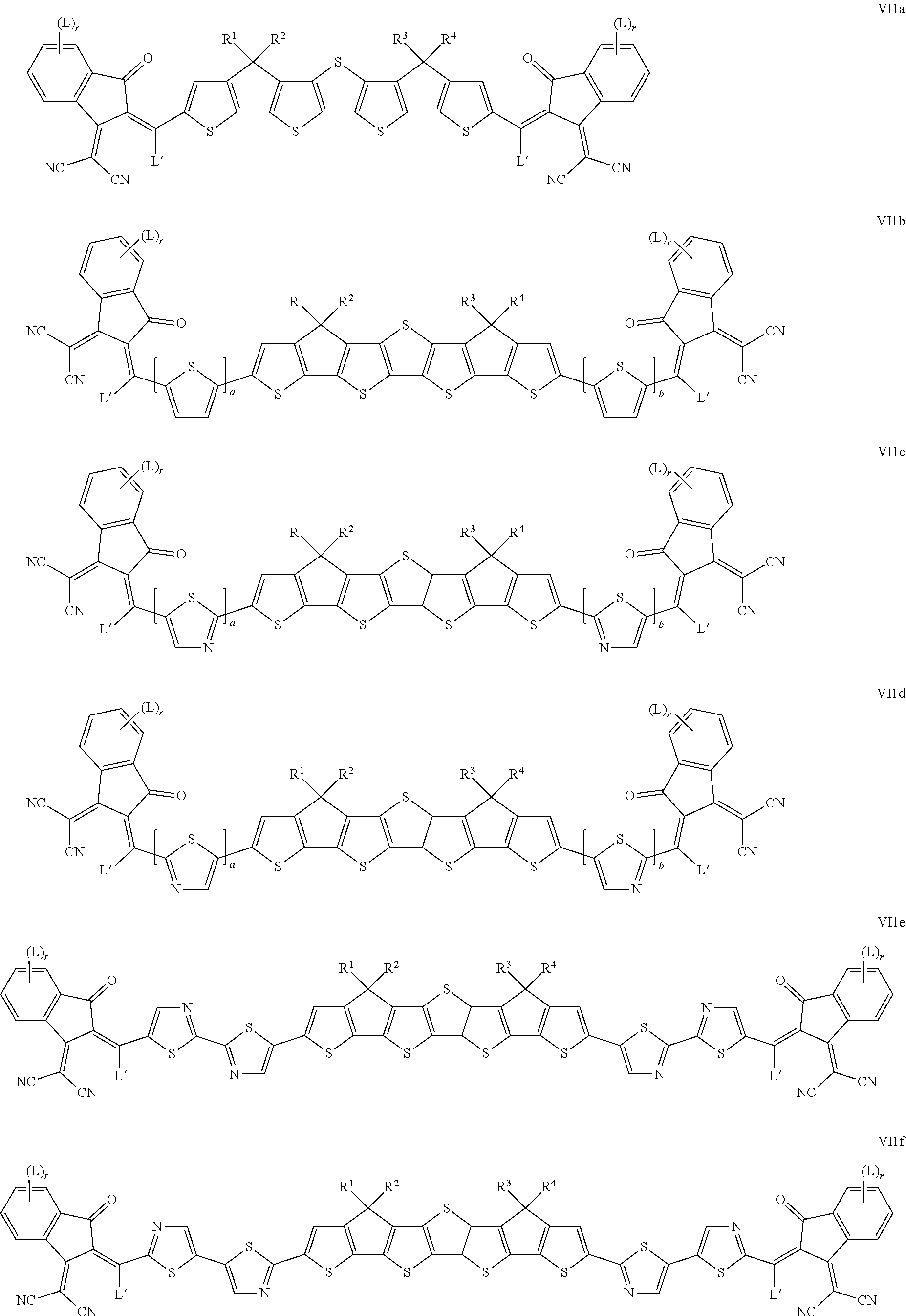

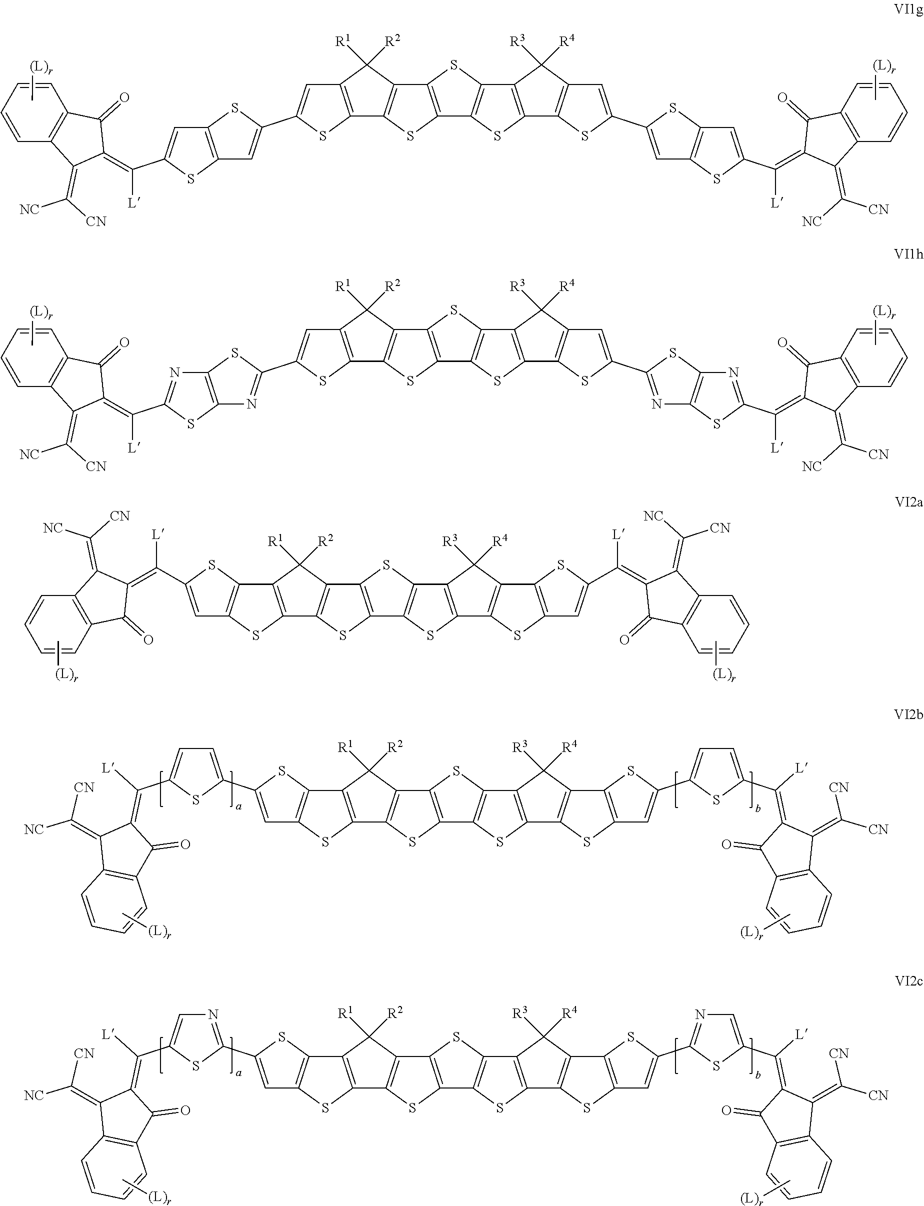

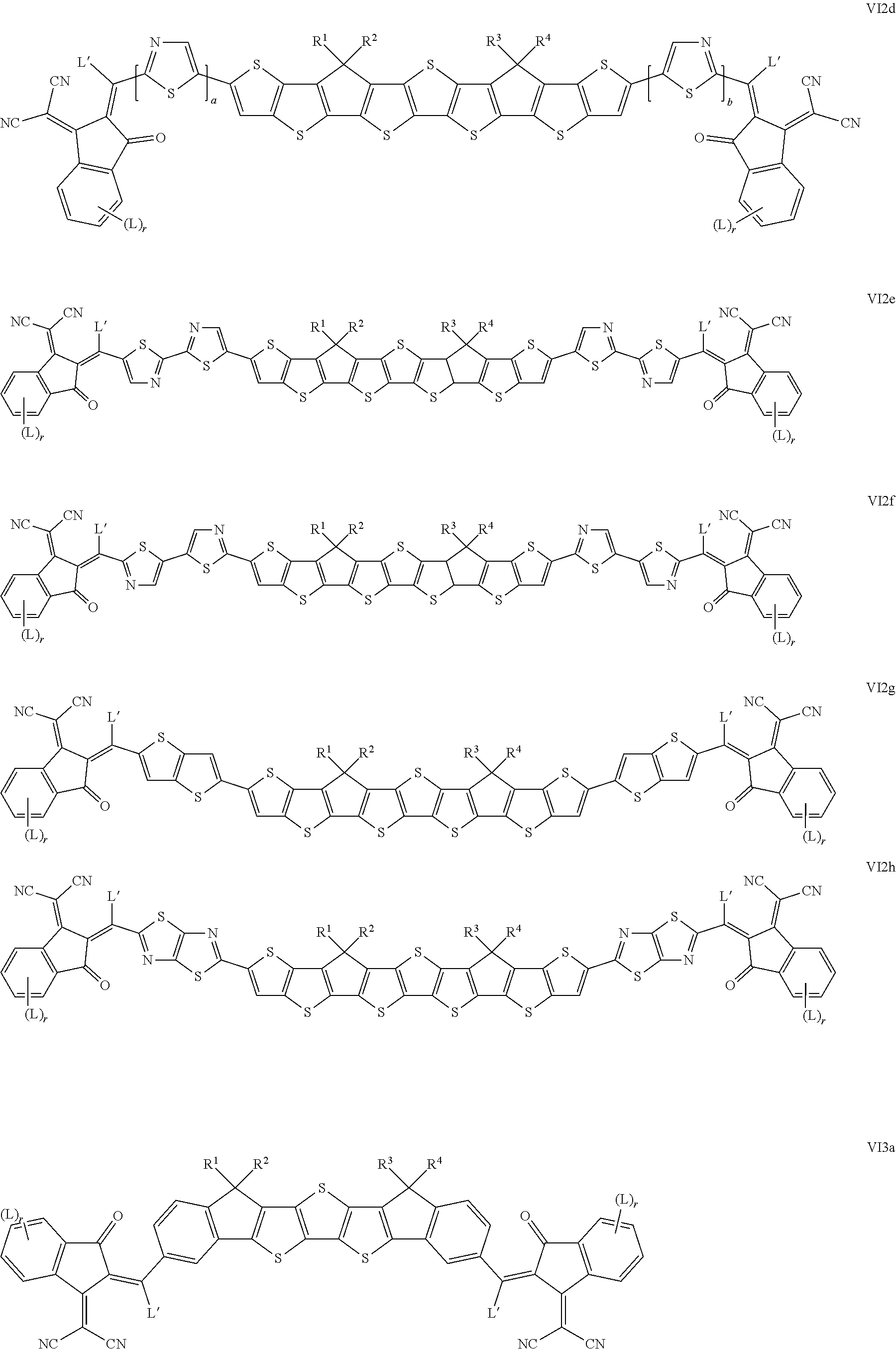

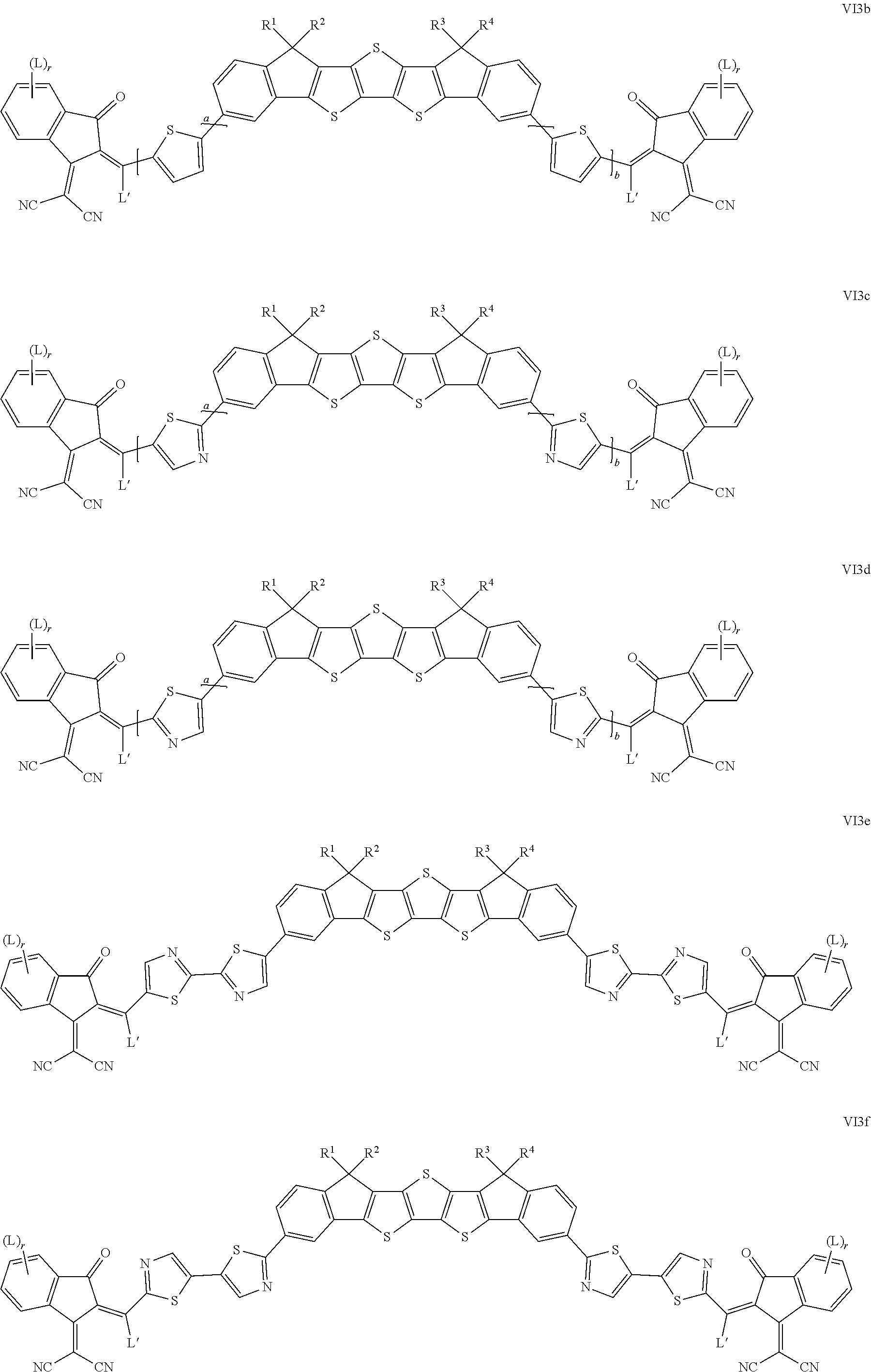

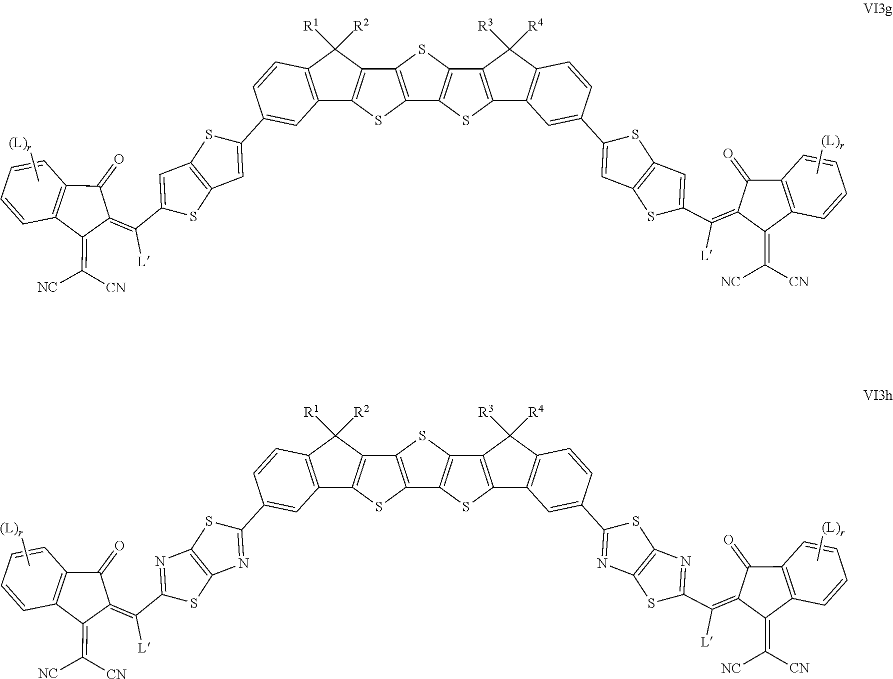

The above formulae VI1a-VI3h do also include their E- or Z-stereoisomers with respect to the C═C double bond of the terminal group in a-position to the adjacent group Ar¹ or Ar⁸, for example the group

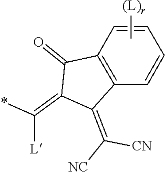

on each occurrence identically or differently may also denote

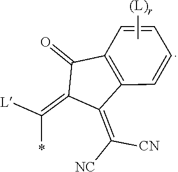

Another preferred embodiment of the present invention relates to a monomer of formula V1 or V2

R²³—(Ar¹)ₐ—U—(Ar²)_{b}—(Ar³)_{c}—(Ar⁴)_{d}—R²⁴ V1

R²³—(Ar¹)ₐ—(Ar²)_{b}—U—(Ar³)_{c}—(Ar⁴)_{d}—R²⁴ V2

wherein U, Ar¹⁻⁴, a, b, c and d have the meanings of formula II1, or one of the preferred meanings as described above and below, and R²³ and R²⁴ are independently of each other selected from the group consisting of H, which is preferably an activated C—H bond, Cl, Br, I, O-tosylate, O-triflate, O-mesylate, O-nonaflate, —SiMe₂F, —SiMeF₂, —O—SO₂Z¹, —B(OZ²)₂, —CZ³═C(Z³)₂, —C≡CH, —C≡CSi(Z¹)₃, —ZnX⁰ and —Sn(Z⁴)₃, wherein X⁰ is halogen, Z¹⁻⁴ are selected from the group consisting of alkyl and aryl, preferably C₁₋₁₀ alkyl and C₆₋₁₂ aryl, each being optionally substituted, and two groups Z² may also form a cycloboronate group having 2 to 20 C atoms together with the B- and O-atoms, and wherein at least one of R²³ and R²⁴ is different from H, and preferably both of R²³ and R²⁴ are different from H.

Very preferred are monomers of formula V1 and V2 and their subformulae wherein a+b+c+d≥1.

Further preferred are monomers of formula V1 and its subformulae wherein a+b+c+d=0.

Further preferred are monomers of formula V1 and V2 and their subformulae wherein R²³ and R²⁴ are selected from Br, —B(OZ²)₂ and Sn(Z⁴)₃.

Further preferred are monomers selected from the following subformulae

R²³—Ar¹—U—Ar²—R²⁴ V1a

R²³—U—R²⁴ V1b

R²³—Ar¹—U—R²⁴ V1c

R²³—U—Ar²—R²⁴ V1d

wherein U, Ar¹, Ar², R²³ and R²⁴ are as defined in formula V1.

Very preferred are monomers of formula V1 and V2 and their subformulae wherein R²³ and R²⁴ are selected from Br, B(OZ²)₂ and Sn(Z⁴)₃.

Further preferred are monomers of formulae V1, V2, V1a-V1d and their subformulae wherein Ar¹ and/or Ar² are selected from the following groups
a) the group consisting of the formulae D1-D145, very preferably of the formulae D1, D7, D10, D11, D19, D22, D29, D30, D35, D36, D37, D44, D55, D84, D87, D88, D89, D93, D94, D106, D111, D139, D140, D141 and D150,
b) the group consisting of the formulae A1-A98, very preferably of the formulae A1, A6, A7, A15, A16, A20, A36, A74, A84, A88, A92, A98 and A103,
c) the group consisting of the formulae Sp1-Sp17, very preferably of the formulae Sp1, Sp2, Sp6, Sp10, Sp11, Sp12, Sp13 and Sp14.

The polymers according to the present invention can be prepared for example by copolymerising one or more monomers of formula V1, V2 or V1a-V1d with each other or with one or monomers of the following formulae in an aryl-aryl coupling reaction

R23-Ar¹—R²⁴ MI

R²³—Ar²—R²⁴ MII

R²³—Ar³—R²⁴ MIII

R²³—Ar⁴—R²⁴ MIV

wherein Ar¹⁻⁴, R²³ and R²⁴ have the meanings given in formula II2 and V1 or one of the preferred meanings given above and below.

The polymer according to the present invention can be synthesized according to or in analogy to methods that are known to the skilled person and are described in the literature. Other methods of preparation can be taken from the examples.

For example, the polymer can be suitably prepared by aryl-aryl coupling reactions, such as Yamamoto coupling, C—H activation coupling, Suzuki coupling, Stille coupling, Sonogashira coupling, Heck coupling or Buchwald coupling. Suzuki coupling, Stille coupling and Yamamoto coupling are especially preferred. The monomers which are polymerised to form the repeat units of the polymers can be prepared according to methods which are known to the person skilled in the art.

Preferably the polymer is prepared from monomers selected from formulae V1, V2, V1a-d and MI-MIV as described above.

Another aspect of the invention is a process for preparing a polymer by coupling one or more identical or different monomers selected from formulae V1, V2, V1a-d with each other and/or with one or more co-monomers, preferably selected from formulae MI-MIV, in a polymerisation reaction, preferably in an aryl-aryl coupling reaction.

Preferred aryl-aryl coupling and polymerisation methods used in the processes described above and below are Yamamoto coupling, Kumada coupling, Negishi coupling, Suzuki coupling, Stille coupling, Sonogashira coupling, Heck coupling, C—H activation coupling, Ullmann coupling or Buchwald coupling. Especially preferred are Suzuki coupling, Negishi coupling, Stille coupling and Yamamoto coupling. Suzuki coupling is described for example in WO 00/53656 A1. Negishi coupling is described for example in J. Chem. Soc., Chem. Commun., 1977, 683-684. Yamamoto coupling is described in for example in T. Yamamoto et al., Prog. Polym. Sci., 1993, 17, 1153-1205, or WO 2004/022626 A1.Stille coupling is described for example in Z. Bao et al., J. Am. Chem. Soc., 1995, 117, 12426-12435. C—H activation is described for example for example in M. Leclerc et al, Angew. Chem. Int. Ed. 2012, 51, 2068-2071. For example, when using Yamamoto coupling, monomers having two reactive halide groups are preferably used. When using Suzuki coupling, monomers having two reactive boronic acid or boronic acid ester groups or two reactive halide groups are preferably used. When using Stille coupling, monomers having two reactive stannane groups or two reactive halide groups are preferably used. When using Negishi coupling, monomers having two reactive organozinc groups or two reactive halide groups are preferably used. When synthesizing a linear polymer by C—H activation polymerisation, preferably a monomer as described above is used wherein at least one reactive group is an activated hydrogen bond.

Preferred catalysts, especially for Suzuki, Negishi or Stille coupling, are selected from Pd(0) complexes or Pd(II) salts. Preferred Pd(0) complexes are those bearing at least one phosphine ligand such as Pd(Ph₃P)₄. Another preferred phosphine ligand is tris(ortho-tolyl)phosphine, i.e. Pd(o-Tol₃P)₄. Preferred Pd(II) salts include palladium acetate, i.e. Pd(Oac)₂ or trans-di(p-acetato)-bis[o-(di-o-tolylphosphino)benzyl]dipalladium(II). Alternatively the Pd(0) complex can be prepared by mixing a Pd(0) dibenzylideneacetone complex, for example tris(dibenzyl-ideneacetone)dipalladium(0), bis(dibenzylideneacetone)palladium(0), or Pd(II) salts e.g. palladium acetate, with a phosphine ligand, for example triphenylphosphine, tris(ortho-tolyl)phosphine, tris(o-methoxyphenyl)phosphine or tri(tert-butyl)phosphine. Suzuki polymerisation is performed in the presence of a base, for example sodium carbonate, potassium carbonate, cesium carbonated, lithium hydroxide, potassium phosphate or an organic base such as tetraethylammonium carbonate or tetraethylammonium hydroxide. Yamamoto polymerisation employs a Ni(0) complex, for example bis(1,5-cyclooctadienyl) nickel(0).

Suzuki, Stille or C—H activation coupling polymerisation may be used to prepare homopolymers as well as statistical, alternating and block random copolymers. Statistical, random block copolymers or block copolymers can be prepared for example from the above monomers, wherein one of the reactive groups is halogen and the other reactive group is a C—H activated bond, boronic acid, boronic acid derivative group or and alkylstannane. The synthesis of statistical, alternating and block copolymers is described in detail for example in WO 03/048225 A2 or WO 2005/014688 A2.

As alternatives to halogen as described above, leaving groups of formula —O—SO₂Z¹ can be used wherein Z¹ is as defined above. Particular examples of such leaving groups are tosylate, mesylate and triflate.

Preferred polymerisation conditions lead to alternating polymers which are particularly preferred for OTFT application, whereas statistical block co-polymers are prepared preferably for OPV and OPD application. Preferred polycondensation are Suzuki coupling, Stille coupling, Sonogashira coupling, Heck coupling or Buchwald coupling, Negishi coupling or C—H activation coupling where the first set of reactive groups is composed of —Cl, —Br, —I, O-tosylate, OP-triflate, O-mesylate and O-nonaflate and the second set of reactive groups is composed of —H, —SiR₂F, —SiRF₂, —B(OR)₂, —CR═CHR′, —C≡CH, —ZnX, —MgX and —Sn(R₃). If a Yamamoto coupling reaction is used to prepare the polymer, the reactive monomer ends are both composed independently of —Cl, —Br, —I, O-tosylate, O-triflate, O-mesylate and O-nonaflate.

Suitable and preferred methods for preparing compounds according to the present invention are illustrated in the reaction schemes below.

Schemes 1-4 show the synthesis of the units of formula I. Therein R and R′ have one of the meanings of R¹ given in formula I, for example alkyl, and Ar is arylene or heteroarylene as defined in formula I.

Scheme 1 exemplarily and schematically illustrates the synthesis of unfunctionalised monomers (Y=Si or Ge, X¹⁻³=S, O or Se).

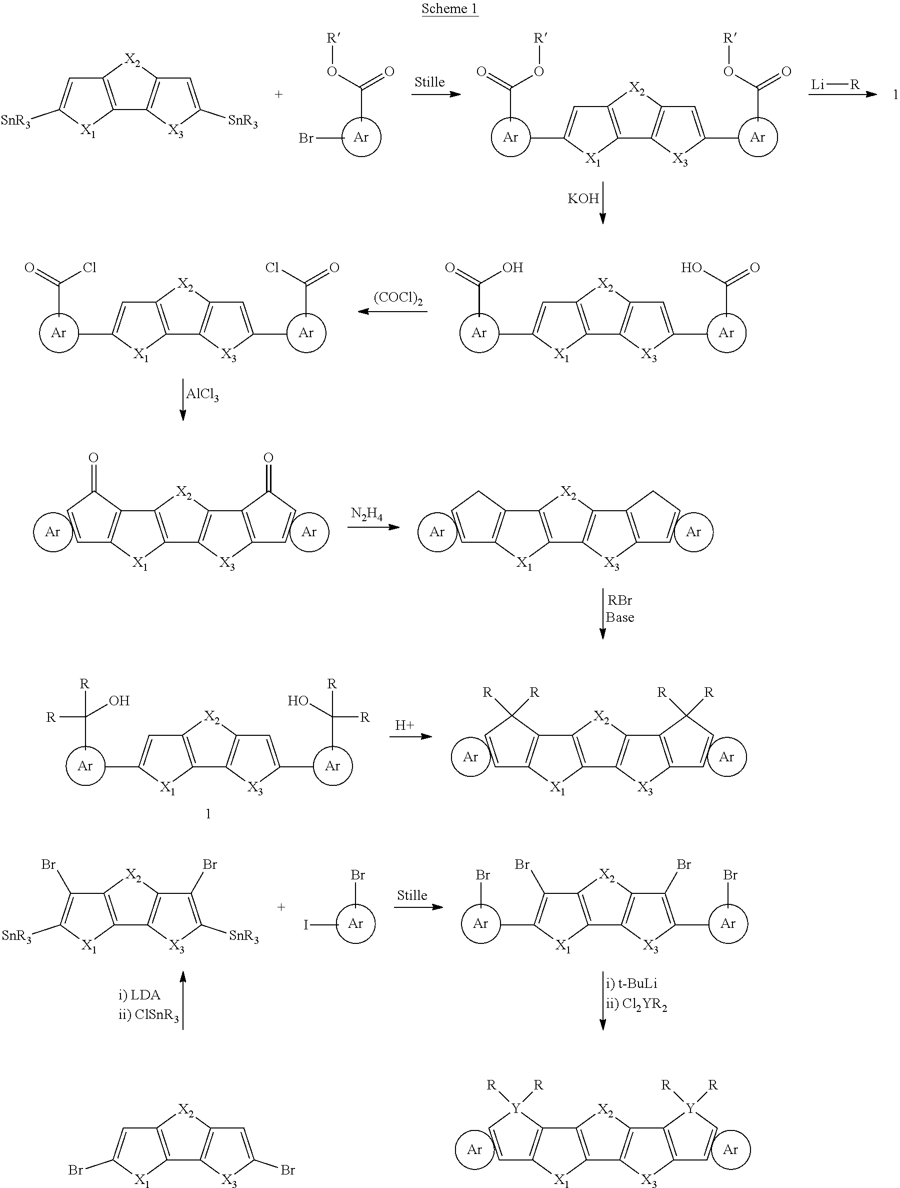

Scheme 2 exemplarily and schematically illustrates the synthesis of functionalised monomers (Y^{1,2}=C, Si or Ge, X¹⁻³=S, O or Se).

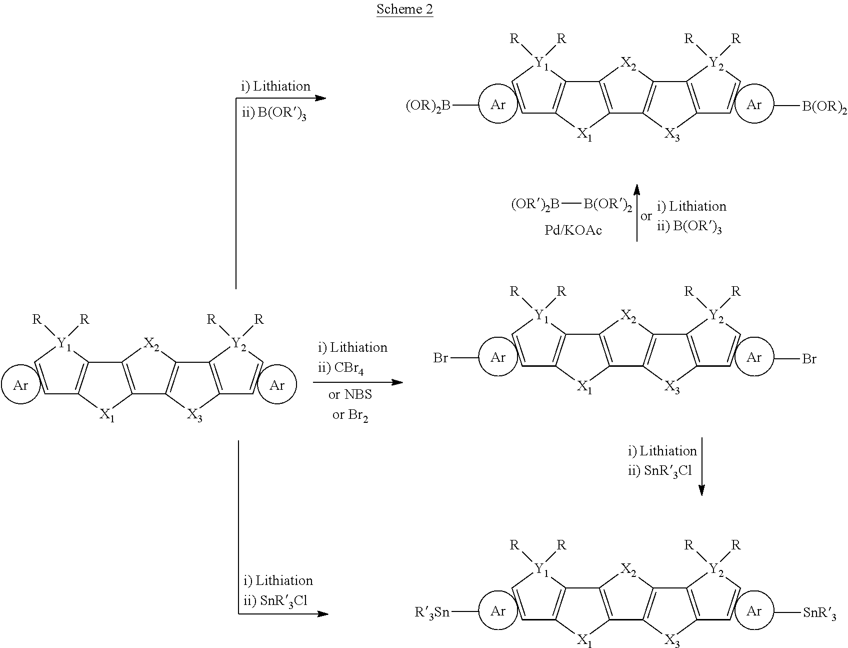

Scheme 3 exemplarily and schematically illustrates the synthesis of a homopolymer.

Scheme 3

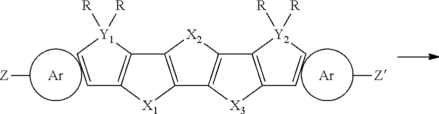

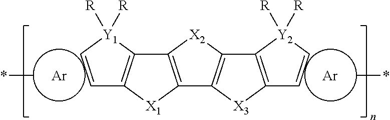

| Z | Z′ | Main Reagent |
|---|---|---|
| H | H | FeCl₃ |
| Br or I | Br or I | Ni(0) |
| SnR′₃ | Br or I | Pd/L |
| B(OR)₂ | Br or I | Pd/L, base |

Scheme 4 exemplarily and schematically illustrates the synthesis of copolymers.

Scheme 4

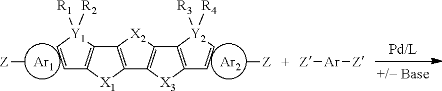

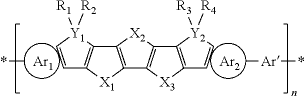

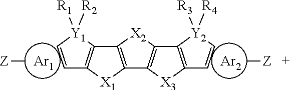

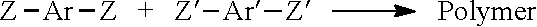

| Z | Z′ |
|---|---|
| Br or I | SnR′₃ |
| Br or I | B(OR′)₂ |
| SnR′₃ | Br or I |
| B(OR′)₂ | Br or I |

The novel methods of preparing a compound, monomer or polymer as described above and below, and the novel monomers and intermediates used therein, are further aspects of the invention.

The compounds according to the present invention can also be used in compositions or polymer blends, for example together with small molecules or other polymers having charge-transport, semiconducting, electrically conducting, photoconducting and/or light-emitting semiconducting properties, or for example with polymers having hole blocking, electron blocking properties for use as interlayers, charge blocking layers, charge transporting layer in OLED devices, OPV devices or perovskite based solar cells.

Small molecules according to the present invention which contain one or more electron withdrawing groups can also be used as n-type semiconductors. For example they can be used as replacement of, or in addition to, fullerenes, especially in mixtures or blends of p-type and n-type semiconductors for use in OPV or OPD devices. Preferred compounds for use as n-type semiconductors are those of formula VI or their subformulae, wherein R^{T1} and/or R^{T2} denote or contain an electron withdrawing group.

Another aspect of the invention relates to a composition, which may also be a polymer blend, comprising one or more compounds according to the present invention and one or more small molecule compounds and/or polymers having one or more of a charge-transport, semiconducting, electrically conducting, photoconducting, hole blocking and electron blocking property.

These compositions can be prepared by conventional methods that are described in prior art and known to the skilled person. Typically the compounds are mixed with each other or dissolved in suitable solvents and the solutions combined.

Another aspect of the invention relates to a formulation comprising one or more polymers, polymer blends or compositions as described above and below and one or more organic solvents.

Preferred solvents are aliphatic hydrocarbons, chlorinated hydrocarbons, aromatic hydrocarbons, ketones, ethers and mixtures thereof. Additional solvents which can be used include 1,2,4-trimethylbenzene, 1,2,3,4-tetra-methyl benzene, pentylbenzene, mesitylene, cumene, cymene, cyclohexylbenzene, diethylbenzene, tetralin, decalin, 2,6-lutidine, 2-fluoro-m-xylene, 3-fluoro-o-xylene, 2-chlorobenzotrifluoride, N,N-dimethylformamide, 2-chloro-6-fluorotoluene, 2-fluoroanisole, anisole, 2,3-dimethylpyrazine, 4-fluoroanisole, 3-fluoroanisole, 3-trifluoro-methylanisole, 2-methylanisole, phenetol, 4-methylanisole, 3-methylanisole, 4-fluoro-3-methylanisole, 2-fluorobenzonitrile, 4-fluoroveratrol, 2,6-dimethylanisole, 3-fluorobenzo-nitrile, 2,5-dimethylanisole, 2,4-dimethylanisole, benzonitrile, 3,5-dimethyl-anisole, N,N-dimethylaniline, ethyl benzoate, 1-fluoro-3,5-dimethoxy-benzene, 1-methylnaphthalene, N-methylpyrrolidinone, 3-fluorobenzo-trifluoride, benzotrifluoride, dioxane, trifluoromethoxy-benzene, 4-fluorobenzotrifluoride, 3-fluoropyridine, toluene, 2-fluoro-toluene, 2-fluorobenzotrifluoride, 3-fluorotoluene, 4-isopropylbiphenyl, phenyl ether, pyridine, 4-fluorotoluene, 2,5-difluorotoluene, 1-chloro-2,4-difluorobenzene, 2-fluoropyridine, 3-chlorofluoro-benzene, 1-chloro-2,5-difluorobenzene, 4-chlorofluorobenzene, chloro-benzene, o-dichlorobenzene, 2-chlorofluorobenzene, p-xylene, m-xylene, o-xylene or mixture of o-, m-, and p-isomers. Solvents with relatively low polarity are generally preferred. For inkjet printing solvents and solvent mixtures with high boiling temperatures are preferred. For spin coating alkylated benzenes like xylene and toluene are preferred.

Examples of especially preferred solvents include, without limitation, dichloromethane, trichloromethane, chlorobenzene, o-dichlorobenzene, tetrahydrofuran, anisole, 2,4-dimethylanisole, 1-methylnaphthalene, morpholine, toluene, o-xylene, m-xylene, p-xylene, 1,4-dioxane, acetone, methylethylketone, 1,2-dichloroethane, 1,1,1-trichloroethane, 1,1,2,2-tetrachloroethane, ethyl acetate, n-butyl acetate, N,N-dimethylformamide, dimethylacetamide, dimethylsulfoxide, 1,5-dimethyltetraline, propiophenone, acetophenone, tetraline, 2-methylthiophene, 3-methylthiophene, decaline, indane, methyl benzoate, ethyl benzoate, mesitylene and/or mixtures thereof.

The concentration of the polymers in the solution is preferably 0.1 to 10% by weight, more preferably 0.5 to 5% by weight. Optionally, the solution also comprises one or more binders to adjust the rheological properties, as described for example in WO 2005/055248 A1.

After the appropriate mixing and ageing, solutions are evaluated as one of the following categories: complete solution, borderline solution or insoluble. The contour line is drawn to outline the solubility parameter-hydrogen bonding limits dividing solubility and insolubility. ‘Complete’ solvents falling within the solubility area can be chosen from literature values such as published in “Crowley, J. D., Teague, G. S. Jr and Lowe, J. W. Jr., Journal of Paint Technology, 1966, 38 (496), 296”. Solvent blends may also be used and can be identified as described in “Solvents, W. H. Ellis, Federation of Societies for Coatings Technology, p 9-10, 1986”. Such a procedure may lead to a blend of ‘non’ solvents that will dissolve both the polymers of the present invention, although it is desirable to have at least one true solvent in a blend.

The compounds according to the present invention can also be used in patterned OSC layers in the devices as described above and below. For applications in modern microelectronics it is generally desirable to generate small structures or patterns to reduce cost (more devices/unit area), and power consumption. Patterning of thin layers comprising a polymer according to the present invention can be carried out for example by photolithography, electron beam lithography or laser patterning.

For use as thin layers in electronic or electrooptical devices the compounds, compositions or formulations according to the present invention may be deposited by any suitable method. Liquid coating of devices is more desirable than vacuum deposition techniques. Solution deposition methods are especially preferred. The formulations of the present invention enable the use of a number of liquid coating techniques. Preferred deposition techniques include, without limitation, dip coating, spin coating, ink jet printing, nozzle printing, letter-press printing, screen printing, gravure printing, doctor blade coating, roller printing, reverse-roller printing, offset lithography printing, dry offset lithography printing, flexographic printing, web printing, spray coating, curtain coating, brush coating, slot dye coating or pad printing.

Ink jet printing is particularly preferred when high resolution layers and devices needs to be prepared. Selected formulations of the present invention may be applied to prefabricated device substrates by ink jet printing or microdispensing. Preferably industrial piezoelectric print heads such as but not limited to those supplied by Aprion, Hitachi-Koki, InkJet Technology, On Target Technology, Picojet, Spectra, Trident, Xaar may be used to apply the organic semiconductor layer to a substrate. Additionally semi-industrial heads such as those manufactured by Brother, Epson, Konica, Seiko Instruments Toshiba TEC or single nozzle microdispensers such as those produced by Microdrop and Microfab may be used.

In order to be applied by ink jet printing or microdispensing, the polymers should be first dissolved in a suitable solvent. Solvents must fulfil the requirements stated above and must not have any detrimental effect on the chosen print head. Additionally, solvents should have boiling points >100° C., preferably >140° C. and more preferably >150° C. in order to prevent operability problems caused by the solution drying out inside the print head. Apart from the solvents mentioned above, suitable solvents include substituted and non-substituted xylene derivatives, di-C₁₋₂-alkyl formamide, substituted and non-substituted anisoles and other phenol-ether derivatives, substituted heterocycles such as substituted pyridines, pyrazines, pyrimidines, pyrrolidinones, substituted and non-substituted N,N-di-C₁₋₂-alkylanilines and other fluorinated or chlorinated aromatics.

A preferred solvent for depositing a compound according to the present invention by ink jet printing comprises a benzene derivative which has a benzene ring substituted by one or more substituents wherein the total number of carbon atoms among the one or more substituents is at least three. For example, the benzene derivative may be substituted with a propyl group or three methyl groups, in either case there being at least three carbon atoms in total. Such a solvent enables an ink jet fluid to be formed comprising the solvent with the compound or polymer, which reduces or prevents clogging of the jets and separation of the components during spraying. The solvent(s) may include those selected from the following list of examples: dodecylbenzene, 1-methyl-4-tert-butylbenzene, terpineol, limonene, isodurene, terpinolene, cymene, diethylbenzene. The solvent may be a solvent mixture, that is a combination of two or more solvents, each solvent preferably having a boiling point >100° C., more preferably >140° C. Such solvent(s) also enhance film formation in the layer deposited and reduce defects in the layer.

The ink jet fluid (that is mixture of solvent, binder and semiconducting compound) preferably has a viscosity at 20° C. of 1-100 mPa·s, more preferably 1-50 mPa·s and most preferably 1-30 mPa·s.

The compounds, compositions and formulations according to the present invention can additionally comprise one or more further components or additives selected for example from surface-active compounds, lubricating agents, wetting agents, dispersing agents, hydrophobing agents, adhesive agents, flow improvers, defoaming agents, deaerators, diluents which may be reactive or non-reactive, auxiliaries, colourants, dyes or pigments, sensitizers, stabilizers, nanoparticles or inhibitors.

The compounds and compositions according to the present invention are useful as charge transport, semiconducting, electrically conducting, photoconducting or light emitting material in optical, electrooptical, electronic, electroluminescent or photoluminescent components or devices. In these devices, a compound or composition of the present invention is typically applied as a thin layer or film.

Thus, the present invention also provides the use of the compound, composition or layer in an electronic device. The formulation may be used as a high mobility semiconducting material in various devices and apparatus. The formulation may be used, for example, in the form of a semiconducting layer or film. Accordingly, in another aspect, the present invention provides a semiconducting layer for use in an electronic device, the layer comprising a polymer, composition or polymer blend according to the invention. The layer or film may be less than about 30 microns. For various electronic device applications, the thickness may be less than about 1 micron thick. The layer may be deposited, for example on a part of an electronic device, by any of the aforementioned solution coating or printing techniques.

The invention additionally provides an electronic device comprising a polymer, polymer blend, composition or organic semiconducting layer according to the present invention. Especially preferred devices are OFETs, TFTs, ICs, logic circuits, capacitors, RFID tags, OLEDs, OLETs, OPEDs, OPVs, OPDs, solar cells, dye-sensitized solar cells (DSSC), perovskite-based solar cells, laser diodes, photoconductors, photodetectors, electrophotographic devices, electrophotographic recording devices, organic memory devices, sensor devices, charge injection layers, Schottky diodes, planarising layers, antistatic films, conducting substrates and conducting patterns.

Especially preferred electronic device are OFETs, OLEDs, OPV and OPD devices, in particular OPD and bulk heterojunction (BHJ) OPV devices. In an OFET, for example, the active semiconductor channel between the drain and source may comprise the layer of the invention. As another example, in an OLED device, the charge (hole or electron) injection or transport layer may comprise the layer of the invention.

For use in OPV or OPD devices the polymer according to the present invention is preferably used in a composition that comprises or contains, preferably consists of, one or more p-type semiconductors and one or more n-type semiconductors.

In a preferred embodiment at least one of the p-type semiconductors in the composition is a compound according to the present invention which is preferably a conjugated polymer. In this preferred embodiment the n-type semiconductor is preferably a fullerene or substituted fullerene.

In another preferred embodiment at least one of the n-type semiconductors in the composition is a compound according to the present invention which is preferably a small molecule, very preferably a compound of formula VI. In this preferred embodiment the p-type semiconductor is preferably a conjugated polymer.

In another preferred embodiment the OPV or OPD device comprises a composition comprising a compound according to the present invention as first n-type semiconductor, and further comprising an p-type semiconductor like a conjugated polymer, and a second n-type semiconductor, which is preferably a fullerene or substituted fullerene.

The n-type semiconductor or second n-type semiconductor in the composition of the aforementioned embodiments is for example an inorganic material such as zinc oxide (ZnOₓ), zinc tin oxide (ZTO), titanium oxide (TiOₓ), molybdenum oxide (MoOₓ), nickel oxide (NiOₓ), or cadmium selenide (CdSe), or an organic material such as graphene or a fullerene, a conjugated polymer or a fullerene or substituted fullerene.

The fullerene is for example an indene-C₆₀-fullerene bisaduct like ICBA, or a (6,6)-phenyl-butyric acid methyl ester derivatized methano C₆₀ fullerene, also known as “PCBM-C₆₀” or “C₆₀PCBM”, as disclosed for example in G. Yu, J. Gao, J. C. Hummelen, F. Wudl, A. J. Heeger, Science 1995, Vol. 270, p. 1789 ff and having the structure shown below, or structural analogous compounds with e.g. a C₆₁ fullerene group, a C₇₀ fullerene group, or a C₇₁ fullerene group, or an organic polymer (see for example Coakley, K. M. and McGehee, M. D. Chem. Mater. 2004, 16, 4533).

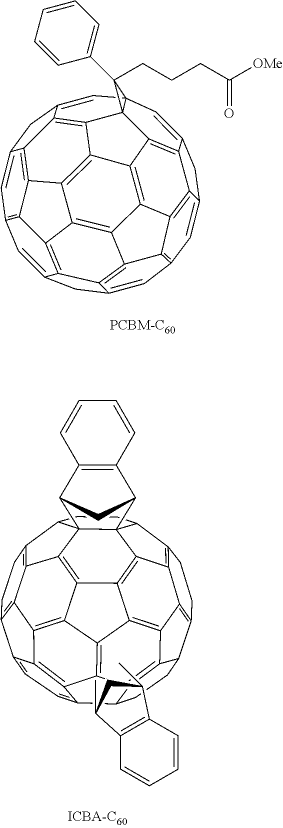

Preferably the fullerene is PCBM-C60, PCBM-C70, bis-PCBM-C60, bis-PCBM-C70, ICMA-c60 (1′,4′-dihydro-naphtho[2′,3′1,2][5,6]fullerene-C60), ICBA, oQDM-C60 (1′,4′-dihydro-naphtho[2′,3′:1,9][5,6]fullerene-C60-lh), or bis-oQDM-C60.

Further preferably the n-type semiconductor or second n-type semiconductor in the composition of the aforementioned embodiments is a fullerene or substituted fullerene of formula XII,

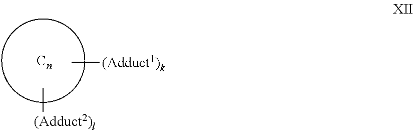

Cₙ denotes a fullerene composed of n carbon atoms, optionally with one or more atoms trapped inside,
Adduct¹ is a primary adduct appended to the fullerene Cₙ with any connectivity,
Adduct² is a secondary adduct, or a combination of secondary adducts, appended to the fullerene Cₙ with any connectivity,
k is an integer ≥1,
and
l is 0, an integer ≥1, or a non-integer >0.

In the formula XII and its subformulae, k preferably denotes 1, 2, 3 or, 4, very preferably 1 or 2.

The fullerene Cₙ in formula XII and its subformulae may be composed of any number n of carbon atoms Preferably, in the compounds of formula XII and its subformulae the number of carbon atoms n of which the fullerene Cₙ is composed is 60, 70, 76, 78, 82, 84, 90, 94 or 96, very preferably 60 or 70.

The fullerene Cₙ in formula XII and its subformulae is preferably selected from carbon based fullerenes, endohedral fullerenes, or mixtures thereof, very preferably from carbon based fullerenes.

Suitable and preferred carbon based fullerenes include, without limitation, (C₆₀₋ₗₕ)[5,6]fullerene, (C_{70-D5h})[5,6]fullerene, (C_{76-D2*})[5,6]fullerene, (C_{84-D2*})[5,6]fullerene, (C_{84-D2d})[5,6]fullerene, or a mixture of two or more of the aforementioned carbon based fullerenes.

The endohedral fullerenes are preferably metallofullerenes. Suitable and preferred metallofullerenes include, without limitation, La@C₆₀, La@C₈₂, Y@C₈₂, Sc₃N@C₈₀, Y₃N@C₈₀, Sc₃C₂@C₈₀ or a mixture of two or more of the aforementioned metallofullerenes.

Preferably the fullerene Cₙ is substituted at a [6,6] and/or [5,6] bond, preferably substituted on at least one [6,6] bond.

Primary and secondary adduct, named “Adduct” in formula XII and its subformulae, is preferably selected from the following formulae

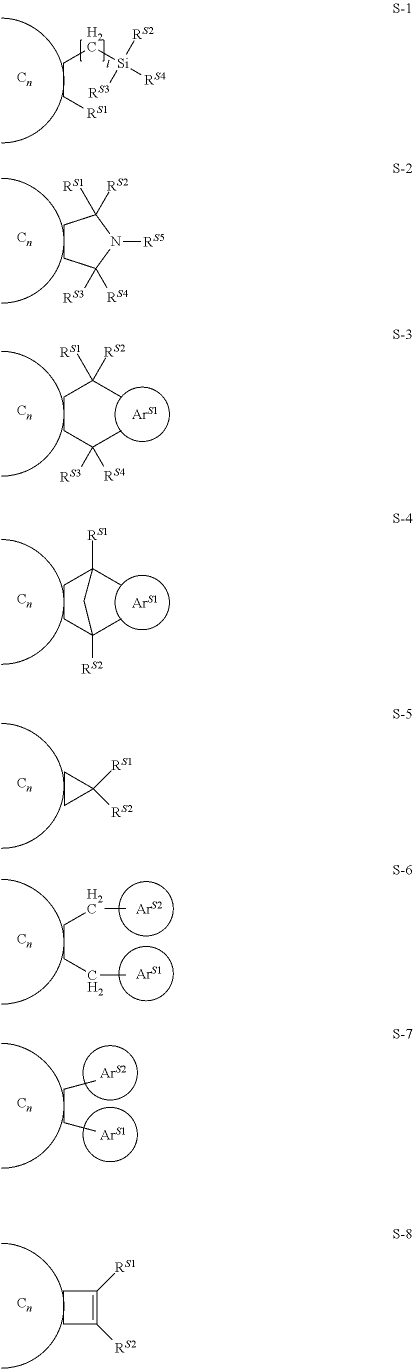

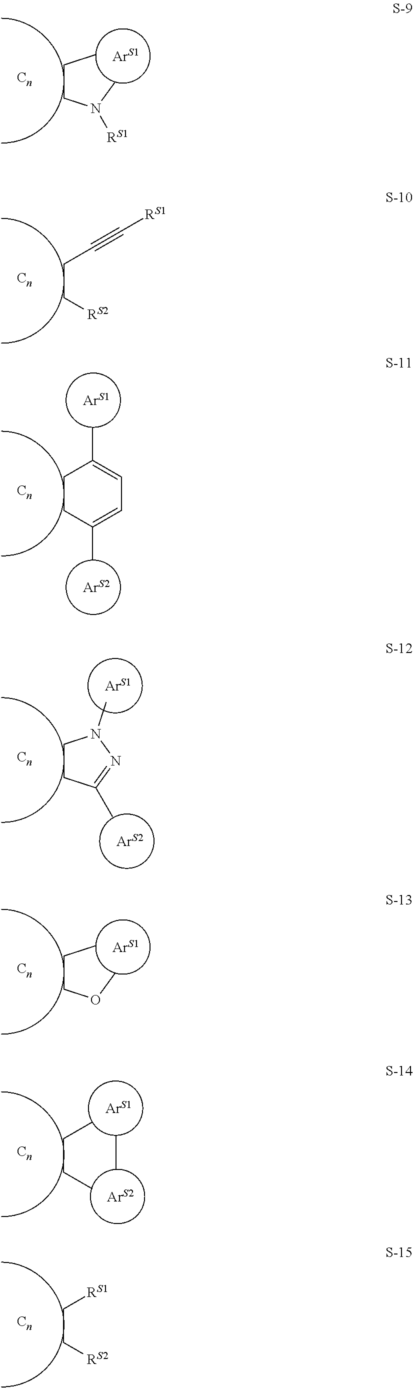

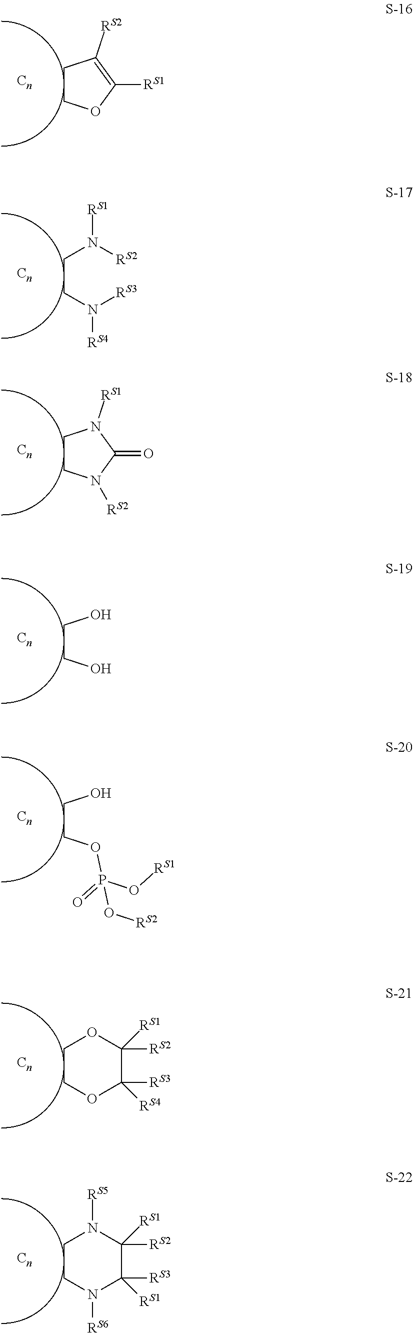

wherein Cₙ is as defined in formula XII,
Ar^{S1}, Ar^{S2} denote, independently of each other, an arylene or heteroarylene group with 5 to 20, preferably 5 to 15, ring atoms, which is mono- or polycyclic, and which is optionally substituted by one or more identical or different substituents having one of the meanings of L as defined above and below, and
R^{S1}, R^{S2}, R^{S3}, R^{S4}, R^{S5} and R^{S6} independently of each other denote H, CN or have one of the meanings of L as defined above and below.

Preferred compounds of formula XII are selected from the following subformulae:

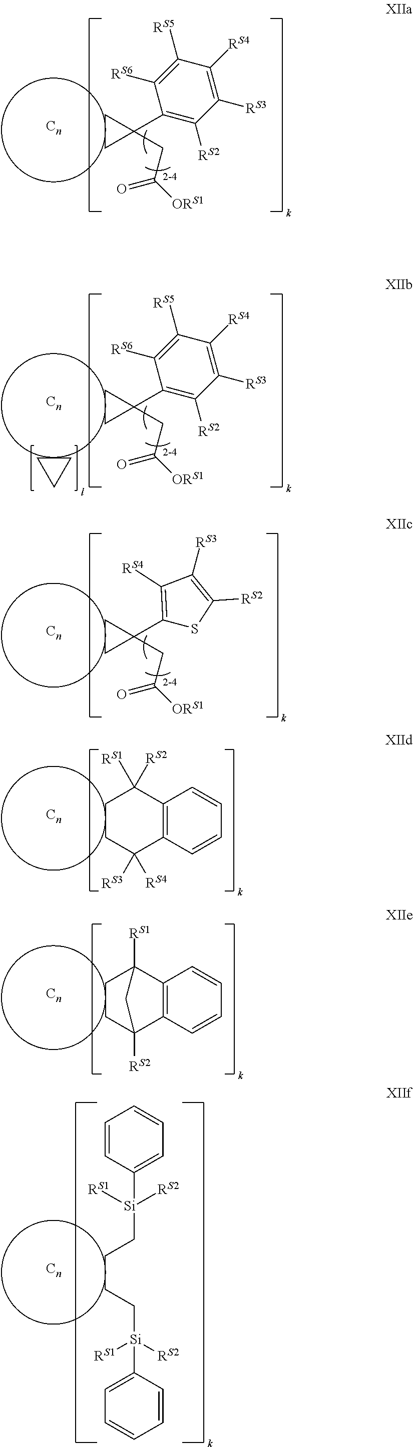

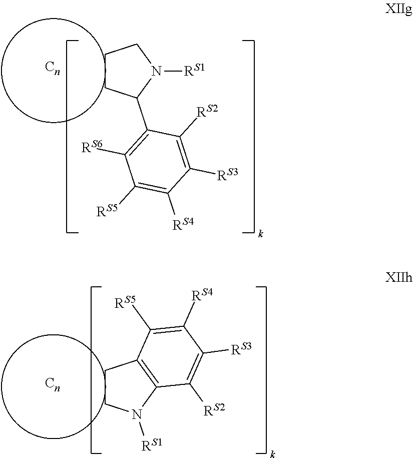

wherein Cₙ, k and l are as defined in formula XII, and
R^{S1}, R^{S2}, R^{S3}, R^{S4} R^{S5} and R^{S6} independently of each other denote H or have one of the meanings of L as defined above and below.

Further preferably the n-type semiconductor or second n-type semiconductor in the composition of the aforementioned embodiments is selected from graphene, metal oxides, like for example, ZnOx, TiOx, ZTO, MoOx, NiOx, quantum dots, like for example, CdSe or CdS, or conjugated polymers, like for example a polynaphthalenediimide or polyperylenediimide as described, for example, in WO2013142841 A1.

The OPV or OPD device according to the present invention preferably comprises a first transparent or semi-transparent electrode on a transparent or semi-transparent substrate on one side of the active layer, and a second metallic or semi-transparent electrode on the other side of the active layer.

Preferably, the photoactive layer in an OPV or OPD device according to the present invention is further blended with additional organic and inorganic compounds to enhance the device properties. For example, metal particles such as Au or Ag nanoparticules or Au or Ag nanoprism for enhancements in light harvesting due to near-field effects (i.e. plasmonic effect) as described, for example in Adv. Mater. 2013, 25 (17), 2385-2396 and Adv. Ener. Mater. 10.1002/aenm.201400206, a molecular dopant such as 2,3,5,6-tetrafluoro-7,7,8,8-tetracyanoquinodimethane for enhancement in photoconductivity as described, for example in Adv. Mater. 2013, 25(48), 7038-7044, or a stabilising agent consisting of a UV absorption agent and/or anti-radical agent and/or antioxidant agent such as 2-hydroxybenzophenone, 2-hydroxyphenylbenzotriazole, oxalic acid anilides, hydroxyphenyl triazines, merocyanines, hindered phenol, N-aryl-thiomorpholine, N-aryl-thiomorpholine-1-oxide, N-aryl-thiomorpholine-1,1-dioxide, N-aryl-thiazolidine, N-aryl-thiazolidine-1-oxide, N-aryl-thiazolidine-1,1-dioxide and 1,4-diazabicyclo[2.2.2]octane as described, for example, in WO2012095796 A1 and in WO2013021971 A1.

The device preferably may further comprise a UV to visible photo-conversion layer such as described, for example, in J. Mater. Chem. 2011, 21, 12331 or a NIR to visible or IR to NIR photo-conversion layer such as described, for example, in J. Appl. Phys. 2013, 113, 124509.

Further preferably the OPV or OPD device comprises, between the active layer and the first or second electrode, one or more additional buffer layers acting as hole transporting layer and/or electron blocking layer, which comprise a material such as metal oxides, like for example, ZTO, MoOₓ, NiOₓ, a doped conjugated polymer, like for example PEDOT:PSS and polypyrrole-polystyrene sulfonate (PPy:PSS), a conjugated polymer, like for example polytriarylamine (PTAA), an organic compound, like for example substituted triaryl amine derivatives such as N,N′-diphenyl-N,N′-bis(1-naphthyl)(1,1′-biphenyl)-4,4′diamine (NPB), N,N′-diphenyl-N,N′-(3-methylphenyl)-1,1′-biphenyl-4,4′-diamine (TPD), graphene based materials, like for example, graphene oxide and graphene quantum dots or alternatively as hole blocking layer and/or electron transporting layer, which comprise a material such as metal oxide, like for example, ZnOₓ, TiOₓ, AZO (aluminium doped zinc oxide), a salt, like for example LiF, NaF, CsF, a conjugated polymer electrolyte, like for example poly[3-(6-trimethylammoniumhexyl)thiophene], poly(9,9-bis(2-ethylhexyl)-fluorene]-b-poly[3-(6-trimethylammoniumhexyl)thiophene], or poly[(9,9-bis(3″-(N,N-dimethylamino)propyl)-2,7-fluorene)-alt-2,7-(9,9-dioctylfluorene)], a polymer, like for example poly(ethyleneimine) or crosslinked N-containing compound derivatives or an organic compound, like for example tris(8-quinolinolato)-aluminium(III) (Alq₃), phenanthroline derivative or C₆₀ or C₇₀ based fullerenes, like for example, as described in Adv. Energy Mater. 2012, 2, 82-86.

In a composition comprising a small molecule compound according to the present invention and further comprising a polymer, the ratio polymer:small molecule compound is preferably from 5:1 to 1:5 by weight, more preferably from 1:1 to 1:3 by weight, most preferably 1:1 to 1:2 by weight.

In a composition comprising a polymer compound according to the present invention and further comprising a fullerene or modified fullerene, the ratio polymer:fullerene is preferably from 5:1 to 1:5 by weight, more preferably from 2:1 to 1:3 by weight, most preferably 1:1 to 1:2 by weight.

The composition according to the present invention may also comprise polymeric binder, preferably from 5 to 95% by weight. Examples of binder include polystyrene (PS), polypropylene (PP), polydimethylsilane (PDMS), and polymethylmethacrylate (PMMA).

To produce thin layers in BHJ OPV devices the compounds, compositions and formulations of the present invention may be deposited by any suitable method. Liquid coating of devices is more desirable than vacuum deposition techniques. Solution deposition methods are especially preferred. The formulations of the present invention enable the use of a number of liquid coating techniques. Preferred deposition techniques include, without limitation, dip coating, spin coating, ink jet printing, nozzle printing, letter-press printing, screen printing, gravure printing, doctor blade coating, roller printing, reverse-roller printing, offset lithography printing, dry offset lithography printing, flexographic printing, web printing, spray coating, curtain coating, brush coating, slot dye coating or pad printing. For the fabrication of OPV devices and modules area printing method compatible with flexible substrates are preferred, for example slot dye coating, spray coating and the like.

In the preparation of a formulation according to the present invention, suitable solvents are preferably selected to ensure full dissolution of both the p-type and n-type component, and take into account the boundary conditions (for example rheological properties) introduced by the chosen printing method.

Organic solvent are generally used for this purpose. Typical solvents can be aromatic solvents, halogenated solvents or chlorinated solvents, including chlorinated aromatic solvents. Examples include, but are not limited to chlorobenzene, 1,2-dichlorobenzene, chloroform, 1,2-dichloroethane, dichloromethane, carbon tetrachloride, toluene, cyclohexanone, ethylacetate, tetrahydrofuran, anisole, 2,4-dimethylanisole, 1-methylnaphthalene, morpholine, toluene, o-xylene, m-xylene, p-xylene, 1,4-dioxane, acetone, methylethylketone, 1,2-dichloroethane, 1,1,1-trichloroethane, 1,1,2,2-tetrachloroethane, ethyl acetate, n-butyl acetate, N,N-dimethylformamide, dimethylacetamide, dimethylsulfoxide, 1,5-dimethyltetraline, propiophenone, acetophenone, tetraline, 2-methylthiophene, 3-methylthiophene, decaline, indane, methyl benzoate, ethyl benzoate, mesitylene and combinations thereof.

The OPV device can for example be of any type known from the literature (see e.g. Waldauf et al., Appl. Phys. Lett., 2006, 89, 233517).

A first preferred OPV device according to the invention comprises the following layers (in the sequence from bottom to top):
optionally a substrate,
a high work function electrode, preferably comprising a metal oxide, like for example ITO and FTO, serving as anode,
an optional conducting polymer layer or hole transport layer, preferably comprising an organic polymer or polymer blend, for example PEDOT:PSS (poly(3,4-ethylenedioxythiophene): poly(styrene-sulfonate), substituted triaryl amine derivatives, for example, TBD (N,N′-dyphenyl-N—N′-bis(3-methylphenyl)-1,1′biphenyl-4,4′-diamine) or NBD (N,N′-dyphenyl-N—N′-bis(1-napthylphenyl)-1,1′biphenyl-4,4′-diamine),
a layer, also referred to as “photoactive layer”, comprising a p-type and an n-type organic semiconductor, which can exist for example as a p-type/n-type bilayer or as distinct p-type and n-type layers, or as blend or p-type and n-type semiconductor, forming a BHJ,
optionally a layer having electron transport properties, for example comprising LiF, TiOₓ, ZnOₓ, PFN, a poly(ethyleneimine) or crosslinked nitrogen containing compound derivatives or a phenanthroline derivatives
a low work function electrode, preferably comprising a metal like for example aluminum, serving as cathode,
   wherein at least one of the electrodes, preferably the anode, is transparent to visible and/or NIR light, and
   wherein the p-type or n-type semiconductor is a compound according to the present invention.

A second preferred OPV device according to the invention is an inverted OPV device and comprises the following layers (in the sequence from bottom to top):
optionally a substrate,
a high work function metal or metal oxide electrode, comprising for example ITO and FTO, serving as cathode, a layer having hole blocking properties, preferably comprising a metal oxide like TiOₓ or ZnOₓ, or comprising an organic compound such as polymer like poly(ethyleneimine) or crosslinked nitrogen containing compound derivatives or phenanthroline derivatives,
a photoactive layer comprising a p-type and an n-type organic semiconductor, situated between the electrodes, which can exist for example as a p-type/n-type bilayer or as distinct p-type and n-type layers, or as blend or p-type and n-type semiconductor, forming a BHJ,
an optional conducting polymer layer or hole transport layer, preferably comprising an organic polymer or polymer blend, for example of PEDOT:PSS, nafion or substituted triaryl amine derivatives, for example TBD or NBD,
an electrode comprising a high work function metal like for example silver, serving as anode,
   wherein at least one of the electrodes, preferably the cathode, is transparent to visible and/or NIR light, and
   wherein the p-type or n-type semiconductor is a compound according to the present invention.

In the OPV devices of the present invention the p-type and n-type semiconductor materials are preferably selected from the materials, like the compound/polymer or compound/polymer/fullerene systems as described above.

When the active layer is deposited on the substrate, it forms a BHJ that phase separates at nanoscale level. For discussion on nanoscale phase separation see Dennler et al, Proceedings of the IEEE, 2005, 93 (8), 1429 or Hoppe et al, Adv. Func. Mater, 2004, 14(10), 1005. An optional annealing step may be then necessary to optimize blend morpohology and consequently OPV device performance.

Another method to optimize device performance is to prepare formulations for the fabrication of OPV(BHJ) devices that may include high boiling point additives to promote phase separation in the right way. 1,8-Octanedithiol, 1,8-diiodooctane, nitrobenzene, 1-chloronaphthalene, N,N-dimethylformamide, dimethylacetamide, dimethylsulfoxide and other additives have been used to obtain high-efficiency solar cells. Examples are disclosed in J. Peet. et al, Nat. Mater., 2007, 6. 497 or Fréchet et al. J. Am. Chem. Soc., 2010, 132, 7595-7597.

Another preferred embodiment of the present invention relates to the use of a compound or composition according to the present invention as dye, hole transport layer, hole blocking layer, electron transport layer and/or electron blocking layer in a DSSC or a perovskite-based solar cells, and to a DSSC or perovskite-based solar cells comprising a compound composition or polymer blend according to the present invention.

DSSCs and perovskite-based DSSCs can be manufactured as described in the literature, for example in Chem. Rev. 2010, 110, 6595-6663, Angew. Chem. Int. Ed. 2014, 53, 2-15 or in WO2013171520A1

The compounds and compositions of the present invention are also suitable for use in the semiconducting channel of an OFET. Accordingly, the invention also provides an OFET comprising a gate electrode, an insulating (or gate insulator) layer, a source electrode, a drain electrode and an organic semiconducting channel connecting the source and drain electrodes, wherein the organic semiconducting channel comprises a compound or composition according to the present invention. Other features of the OFET are well known to those skilled in the art.

OFETs where an OSC material is arranged as a thin film between a gate dielectric and a drain and a source electrode, are generally known, and are described for example in U.S. Pat. Nos. 5,892,244, 5,998,804, 6,723,394 and in the references cited in the background section. Due to the advantages, like low cost production using the solubility properties of the compounds according to the invention and thus the processibility of large surfaces, preferred applications of these FETs are such as integrated circuitry, TFT displays and security applications.

The gate, source and drain electrodes and the insulating and semiconducting layer in the OFET device may be arranged in any sequence, provided that the source and drain electrode are separated from the gate electrode by the insulating layer, the gate electrode and the semiconductor layer both contact the insulating layer, and the source electrode and the drain electrode both contact the semiconducting layer.

An OFET device according to the present invention preferably comprises:
a source electrode,
a drain electrode,
a gate electrode,
a semiconducting layer,
one or more gate insulator layers,
optionally a substrate.
   wherein the semiconductor layer comprises a compound or composition according to the present invention.

The OFET device can be a top gate device or a bottom gate device. Suitable structures and manufacturing methods of an OFET device are known to the skilled in the art and are described in the literature, for example in US 2007/0102696 A1.

The gate insulator layer preferably comprises a fluoropolymer, like e.g. the commercially available Cytop 809M® or Cytop 107M® (from Asahi Glass). Preferably the gate insulator layer is deposited, e.g. by spin-coating, doctor blading, wire bar coating, spray or dip coating or other known methods, from a formulation comprising an insulator material and one or more solvents with one or more fluoro atoms (fluorosolvents), preferably a perfluorosolvent. A suitable perfluorosolvent is e.g. FC75® (available from Acros, catalogue number 12380). Other suitable fluoropolymers and fluorosolvents are known in prior art, like for example the perfluoropolymers Teflon AF® 1600 or 2400 (from DuPont) or Fluoropel® (from Cytonix) or the perfluorosolvent FC 43® (Acros, No. 12377). Especially preferred are organic dielectric materials having a low permittivity (or dielectric contant) from 1.0 to 5.0, very preferably from 1.8 to 4.0 (“low k materials”), as disclosed for example in US 2007/0102696 A1 or U.S. Pat. No. 7,095,044.

In security applications, OFETs and other devices with semiconducting materials according to the present invention, like transistors or diodes, can be used for RFID tags or security markings to authenticate and prevent counterfeiting of documents of value like banknotes, credit cards or ID cards, national ID documents, licenses or any product with monetry value, like stamps, tickets, shares, cheques etc.

Alternatively, the compounds and compositions according to the invention can be used in OLEDs, e.g. as the active display material in a flat panel display applications, or as backlight of a flat panel display like e.g. a liquid crystal display. Common OLEDs are realized using multilayer structures. An emission layer is generally sandwiched between one or more electron-transport and/or hole-transport layers. By applying an electric voltage electrons and holes as charge carriers move towards the emissive layer where their recombination leads to the excitation and hence luminescence of the lumophor units contained in the emission layer.

The compounds and compositions according to the invention can be employed in one or more of a buffer layer, electron or hole transport layer, electron or hole blocking layer and emissive layer, corresponding to their electrical and/or optical properties. Furthermore their use within the emissive layer is especially advantageous, if the compounds according to the invention show electroluminescent properties themselves or comprise electroluminescent groups or compounds. The selection, characterization as well as the processing of suitable monomeric, oligomeric and polymeric compounds or materials for the use in OLEDs is generally known by a person skilled in the art, see, e.g., Müller et al, Synth. Metals, 2000, 111-112, 31-34, Alcala, J. Appl. Phys., 2000, 88, 7124-7128 and the literature cited therein.

According to another use, the compounds and compositions according to the present invention, especially those showing photoluminescent properties, may be employed as materials of light sources, e.g. in display devices, as described in EP 0 889 350 A1 or by C. Weder et al., Science, 1998, 279, 835-837.

A further aspect of the invention relates to both the oxidised and reduced form of a compound according to the present invention. Either loss or gain of electrons results in formation of a highly delocalised ionic form, which is of high conductivity. This can occur on exposure to common dopants. Suitable dopants and methods of doping are known to those skilled in the art, e.g. from EP 0 528 662, U.S. Pat. No. 5,198,153 or WO 96/21659.

The doping process typically implies treatment of the semiconductor material with an oxidating or reducing agent in a redox reaction to form delocalised ionic centres in the material, with the corresponding counterions derived from the applied dopants. Suitable doping methods comprise for example exposure to a doping vapor in the atmospheric pressure or at a reduced pressure, electrochemical doping in a solution containing a dopant, bringing a dopant into contact with the semiconductor material to be thermally diffused, and ion-implantantion of the dopant into the semiconductor material.

When electrons are used as carriers, suitable dopants are for example halogens (e.g., I₂, Cl₂, Br₂, ICl, ICl₃, IBr and IF), Lewis acids (e.g., PF₅, AsF₅, SbF₅, BF₃, BCl₃, SbCl₅, BBr₃ and SO₃), protonic acids, organic acids, or amino acids (e.g., HF, HCl, HNO₃, H₂SO₄, HClO₄, FSO₃H and ClSO₃H), transition metal compounds (e.g., FeCl₃, FeOCl, Fe(ClO₄)₃, Fe(4-CH₃C₆H₄SO₃)₃, TiCl₄, ZrCl₄, HfCl₄, NbF₅, NbCl₅, TaCl₅, MoF₅, MoCl₅, WF₅, WCl₆, UF₆ and LnCl₃ (wherein Ln is a lanthanoid), anions (e.g., Cl⁻, Br⁻, I⁻, I₃⁻, HSO₄⁻, SO₄²⁻, NO₃⁻, ClO₄⁻, BF₄⁻, PF₆⁻, AsF₆⁻, SbF₆⁻, FeCl₄⁻, Fe(CN)₆³⁻, and anions of various sulfonic acids, such as aryl-SO₃⁻). When holes are used as carriers, examples of dopants are cations (e.g., H⁺, Li⁺, Na⁺, K⁺, Rb⁺ and Cs⁺), alkali metals (e.g., Li, Na, K, Rb, and Cs), alkaline-earth metals (e.g., Ca, Sr, and Ba), O₂, XeOF₄, (NO₂⁺) (SbF₆⁻), (NO₂⁺) (SbCl₆⁺), (NO₂⁺)(BF₄⁻), AgClO₄, H₂IrCl₆, La(NO₃)₃.6H₂O, FSO₂OOSO₂F, Eu, acetylcholine, R₄N⁺, (R is an alkyl group), R₄P⁺ (R is an alkyl group), R₆As⁺ (R is an alkyl group), and R₃S⁺ (R is an alkyl group).

The conducting form of a compound according to the present invention can be used as an organic “metal” in applications including, but not limited to, charge injection layers and ITO planarising layers in OLED applications, films for flat panel displays and touch screens, antistatic films, printed conductive substrates, patterns or tracts in electronic applications such as printed circuit boards and condensers.

The compounds and compositions according to the present invention may also be suitable for use in organic plasmon-emitting diodes (OPEDs), as described for example in Koller et al., Nat. Photonics, 2008, 2, 684.

According to another use, the compounds according to the present invention can be used alone or together with other materials in or as alignment layers in LCD or OLED devices, as described for example in US 2003/0021913. The use of charge transport compounds according to the present invention can increase the electrical conductivity of the alignment layer. When used in an LCD, this increased electrical conductivity can reduce adverse residual dc effects in the switchable LCD cell and suppress image sticking or, for example in ferroelectric LCDs, reduce the residual charge produced by the switching of the spontaneous polarisation charge of the ferroelectric LCs. When used in an OLED device comprising a light emitting material provided onto the alignment layer, this increased electrical conductivity can enhance the electroluminescence of the light emitting material. The compounds according to the present invention having mesogenic or liquid crystalline properties can form oriented anisotropic films as described above, which are especially useful as alignment layers to induce or enhance alignment in a liquid crystal medium provided onto said anisotropic film. The polymers according to the present invention may also be combined with photoisomerisable compounds and/or chromophores for use in or as photoalignment layers, as described in US 2003/0021913 A1.

According to another use the compounds and compositions according to the present invention, especially their water-soluble derivatives (for example with polar or ionic side groups) or ionically doped forms, can be employed as chemical sensors or materials for detecting and discriminating DNA sequences. Such uses are described for example in L. Chen, D. W. McBranch, H. Wang, R. Helgeson, F. Wudl and D. G. Whitten, Proc. Natl. Acad. Sci. U.S.A., 1999, 96, 12287; D. Wang, X. Gong, P. S. Heeger, F. Rininsland, G. C. Bazan and A. J. Heeger, Proc. Natl. Acad. Sci. U.S.A., 2002, 99, 49; N. DiCesare, M. R. Pinot, K. S. Schanze and J. R. Lakowicz, Langmuir, 2002, 18, 7785; D. T. McQuade, A. E. Pullen, T. M. Swager, Chem. Rev., 2000, 100, 2537.

Unless the context clearly indicates otherwise, as used herein plural forms of the terms herein are to be construed as including the singular form and vice versa.

Throughout the description and claims of this specification, the words “comprise” and “contain” and variations of the words, for example “comprising” and “comprises”, mean “including but not limited to”, and are not intended to (and do not) exclude other components.

It will be appreciated that variations to the foregoing embodiments of the invention can be made while still falling within the scope of the invention. Each feature disclosed in this specification, unless stated otherwise, may be replaced by alternative features serving the same, equivalent or similar purpose. Thus, unless stated otherwise, each feature disclosed is one example only of a generic series of equivalent or similar features.

All of the features disclosed in this specification may be combined in any combination, except combinations where at least some of such features and/or steps are mutually exclusive. In particular, the preferred features of the invention are applicable to all aspects of the invention and may be used in any combination. Likewise, features described in non-essential combinations may be used separately (not in combination).

Above and below, unless stated otherwise percentages are percent by weight and temperatures are given in degrees Celsius. Values of the di-electric constant ε (“permittivity”) refer to values taken at 20° C. and 1,000 Hz.

The invention will now be described in more detail by reference to the following examples, which are illustrative only and do not limit the scope of the invention.

### Example 1

### Dimethyl-6,6′-(dithieno[3,2-b:2′,3′-d]thiophene-2,6-diyl)bis(3-bromobenzoate)

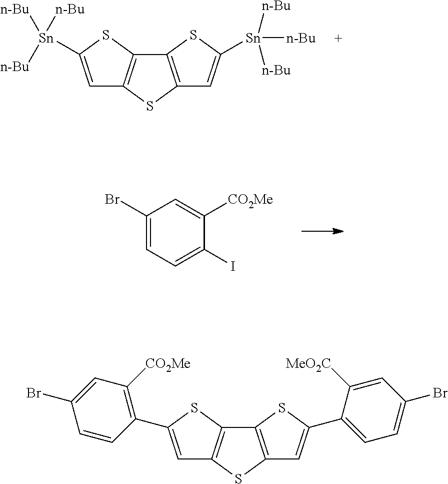

To a degassed mixture of 2,6-bis(tributylstannyl)dithieno[3,2-b:2′,3′-d]thiophene (10 g, 12.9 mmol), methyl 5-bromo-2-iodobenzoate (10.1 g, 29.7 mmol) and anhydrous toluene (200 cm³) was added tri-o-tolyl phosphine (0.1 g, 0.32 mmol) and bis(triphenylphosphine)palladium (II) dichloride (0.12 g, 0.17 mmol). The mixture was further degassed for 10 minutes and then heated at 80° C. for 21 hours. After cooling to 23° C., the reaction mixture was poured into water (250 cm³) and the organic layer decanted. The organic layer was washed with brine (200 cm³), dried over anhydrous magnesium sulphate, filtered and the solvent removed in vacuo. The residue was triturated in acetonitrile and the solid collected by filtration. The solid was purified by recrystallisation (acetonitrile:dichloromethane; 1:1) to give 1.5 g of product. Filtrates from the trituration and recrystallisation were combined and purified by silica gel chromatography (dichloromethane:heptanes; 3:2) to give further product. Both portions were combined to give dimethyl-6,6′-(dithieno[3,2-b:2′,3′-d]thiophene-2,6-diyl)bis(3-bromobenzoate) (1.75 g, 40%) as a yellow solid. ¹H-NMR (400 MHz, CDCl₃) 7.93 (2H, d, J 2.4), 7.68 (2H, dd, J 2.8, 7.9), 7.43 (2H, d, J 8.3), 7.26 (2H, s), 3.80 (6H, s).

### (Dithieno[3,2-b:2′,3′-d]thiophene-2,6-diylbis(3-bromo-6,1-phenylene))bis(bis(4-hexadecylphenyl)methanol)

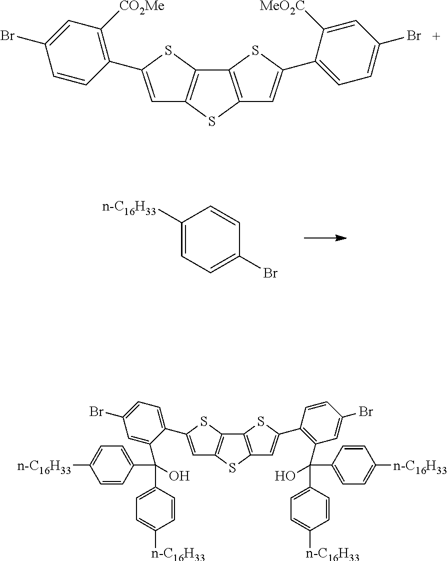

To a mixture of 1-bromo-4-hexadecylbenzene (9.8 g, 25.7 mmol) and anhydrous tetrahydrofuran (140 cm³) at −65° C. was added n-butyllithium (11 cm³, 27.5 mmol, 2.5 M in hexanes) dropwise over a period of 20 minutes. The resulting suspension was stirred at −65° C. for 3.5 hours before dimethyl 6,6′-(dithieno[3,2-b:2′,3′-d]thiophene-2,6-diyl)bis(3-bromobenzoate) (3.20 g, 5.1 mmol) was added. The reaction mixture was stirred and warmed slowly over 17 hours to 23° C. Water (100 cm³) and tert-butyl methyl ether (100 cm³) were added and the mixture stirred for 30 minutes. The organic layer was decanted and the aqueous layer extracted with tert-butyl methyl ether (3×50 cm³). The combined organics dried over anhydrous magnesium sulphate, filtered and the solvent removed in vacuo. The residue purified by silica gel chromatography (heptanes:ethyl acetate; 19:1) to give (dithieno[3,2-b:2′,3′-d]thiophene-2,6-diylbis(3-bromo-6,1-phenylene))bis(bis(4-hexadecylphenyl)methanol) (6.9 g, 76%) as an orange oil. ¹H-NMR (400 MHz, CDCl₃) 7.46 (2H, dd, J 2.3, 8.1), 7.21 (2H, d, J 8.1), 7.05-7.13 (18H, m), 6.16 (2H, s), 3.27 (2H, s), 2.63 (8H, m), 1.63 (8H, m), 1.27-1.34 (104H, m), 0.89 (12H, t, J 6.8).

Compound 1

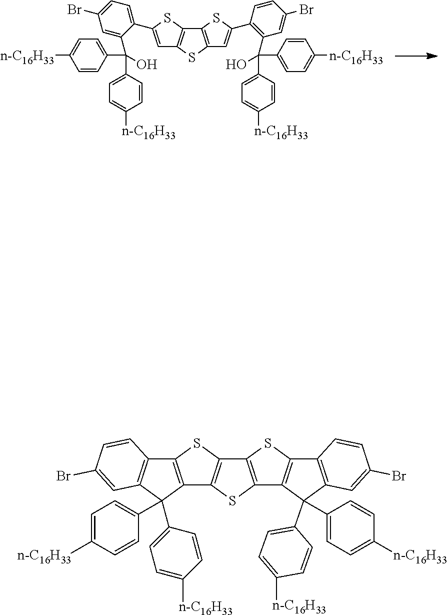

To a mixture of (dithieno[3,2-b:2′,3′-d]thiophene-2,6-diylbis(3-bromo-6,1-phenylene))bis(bis(4-hexadecyl phenyl)methanol) (6.9 g, 3.9 mmol) and dichloromethane (180 cm³) was added p-toluene sulfonic acid (1.48 g, 7.8 mmol) and the reaction mixture heated at reflux for 5 hours. The mixture cooled to 23° C. and the solvent removed in vacuo. The residue purified by silica pad (heptanes) and recrystallisation (2-butanone) to give compound 1 (2.6 g, 38%) as a yellow solid. ¹H-NMR (400 MHz, CDCl₃) 7.51 (2H, d, J 1.8), 7.45 (2H, dd, J 1.8, 8.1), 7.31 (2H, d, J 8.1), 2.53 (8H, m), 1.52-1.62 (8H, m), 1.20-1.39 (104H, m), 0.89 (12H, m).

Polymer 1

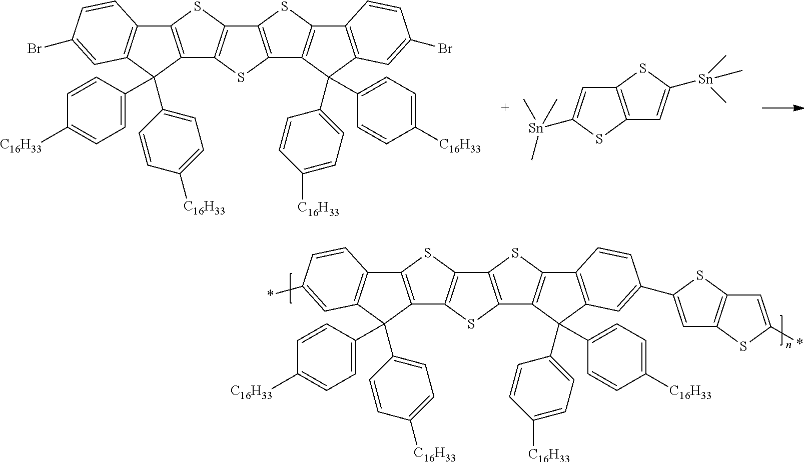

To mixture of compound 1 (500.0 mg, 0.289 mmol), 2,5-bis-trimethylstannanyl-thieno[3,2-b]thiophene (134.4, 0.289 mmol), tris(dibenzylideneacetone)dipalladium(0) (4.1 mg, 0.0058 mmol), tri-o-tolyl phosphine (7.0 mg, 0.023 mmol), toluene (15.0 cm³) and N,N-dimethylformamide (3.0 cm³) was degassed by bubbling nitrogen for 30 minutes. The reaction mixture was then heated at 110° C. under nitrogen in a pre-heated block for 17 hours. Bromobenzene (0.03 cm²) was added and the mixture heated at 110° C. for 20 minutes. Phenytributyltin (0.19 cm³) was added and the mixture heated at 110° C. for 20 minutes. The mixture allowed to cool slightly and then precipitated into stirred methanol (150 cm³). The solid collected by filtration and subjected to Soxhlet extraction: acetone, 40-60 petrol, cyclohexane and chloroform. The chloroform extract was poured into stirred methanol (500 cm³) and the solid collected by filtration to give polymer 1 (469 mg, 95%) as an orange solid. GPC (chlorobenzene, 50° C.) Mₙ=21,400 g/mol, M_{w}=76,900 g/mol.

### Example 2

Compound 2

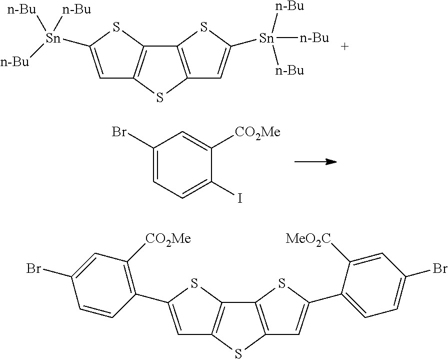

A mixture of 2,6-bis(tributylstannyl)dithieno[3,2-b:2′,3′-d]thiophene (6.5 g, 8.4 mmol), 2-bromo-3-thiophenecarboxylic acid methyl ester (4.1 g, 18 mmol), tri-o-tolylphosphine (200 mg, 0.67 mmol), N,N-dimethylformamide (30 cm³) and anhydrous toluene (150 cm³) was degassed for 25 minutes. Bis(triphenylphosphine)palladium (II) dichloride (118 mg, 0.17 mmol) was added and the mixture stirred at a gentle reflux for 17 hours. The reaction mixture was allowed to cool to 23° C. and poured into methanol (500 cm³). The precipitate collected by filtration and purified by silica gel chromatography (gradient from 40-60 petrol to dichloromethane) to give compound 2 (1.26 g, 32%) as a yellow solid. ¹H-NMR (300 MHz, CDCl₃) 7.75 (2H, s), 7.52 (2H, d, J 5.5), 7.24 (2H, d, J 5.5), 3.87 (6H, s).

Compound 3

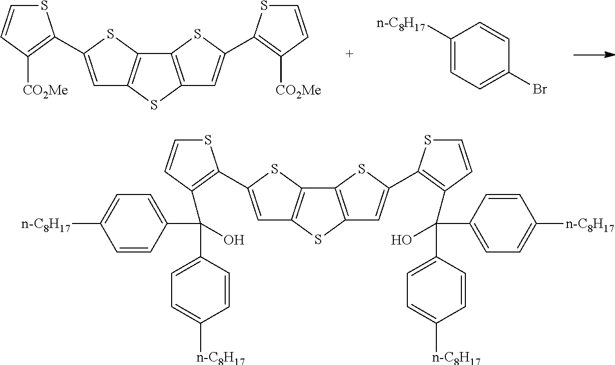

To a solution of 1-bromo-4-octylbenzene (3.56 g, 13 mmol) in anhydrous tetrahydrofuran (150 cm³) at −78° C. was added dropwise n-butyllithium (5.8 cm³, 15 mmol, 2.5 M in hexanes) over 30 minutes. The solution was then stirred at −78° C. for 1 hour before addition of compound 2 (1.26 g, 2.6 mmol). The mixture was stirred for 5 minutes, the cooling removed and the mixture stirred at 23° C. for 17 hours. Water (50 cm³) was added to the reaction mixture and the organic was extracted with ether (100 cm³). The organic layer was dried over anhydrous magnesium sulfate, filtered and the solvent removed in vacuo to give crude compound 3 (3.1 g) as a brown/yellow oil. ¹H-NMR (300 MHz, CDCl₃) 7.24-7.29 (2H, m), 7.07-7.21 (16H, m), 6.72 (2H, s), 6.49 (2H, d, J 5.3), 3.22 (2H, s), 2.52-2.64 (8H, m), 1.50-1.66 (8H, m), 1.13-1.39 (40H, m), 0.79-0.93 (12H, m).

Compound 4

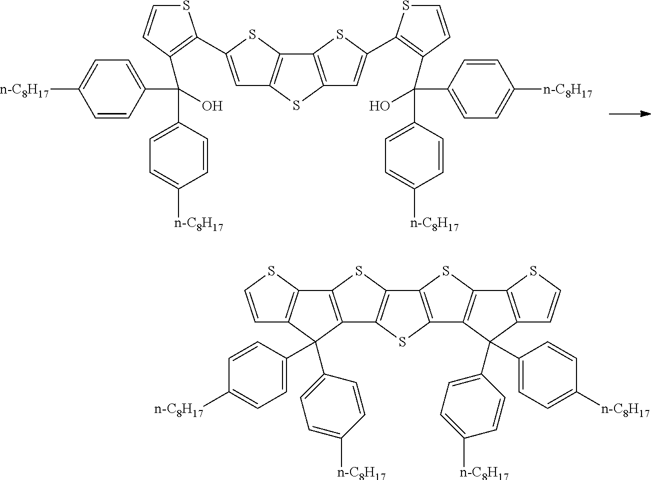

To a solution of crude compound 3 (3.1 g, 2.6 mmol) in dichloromethane (100 cm³) was added p-toluenesulfonic acid (0.91 g, 5.3 mmol), the mixture heated at reflux for 4 hours and at 30° C. for 17 hours. The mixture allowed to cool to 23° C. and passed through a short silica pad (dichloromethane). The residue purified by silica gel chromatography (gradient from 40-60 petrol to 40-60 petrol:dichloromethane; 9:1) to give compound 4 (430 mg, 14%) as a yellow solid. ¹H-NMR (300 MHz, CDCl₃) 7.19 (2H, d, J 5.0), 7.09-7.15 (8H, m), 7.19 (2H, d, J 5.0), 6.99-7.04 (8H, m), 2.44-2.58 (8H, m), 1.48-1.62 (8H, m), 1.16-1.37 (40H, m), 0.81-0.92 (12H, m).

Compound 5

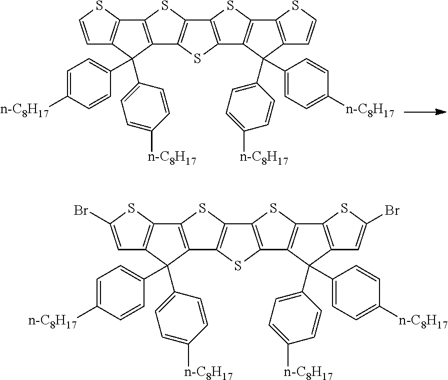

To compound 4 (430 mg, 0.38 mmol) in anhydrous tetrahydrofuran (30 cm³) at 23° C. was added N-bromosuccinimide (138 mg, 0.77 mmol). The reaction mixture was then stirred at 23° C. for 3 hours. Water (50 cm³) was added and the organics extracted with ether (3×50 cm³). The combined organics dried over anhydrous magnesium sulfate, filtered and the solvent removed in vacuo. The residue purified by silica gel chromatography (gradient from 40-60 petrol to 40-60 petrol:dichloromethane; 19:1) to give compound 5 (380 mg, 78%) as a yellow solid. ¹H-NMR (300 MHz, CDCl₃) 7.05-7.11 (8H, m), 7.05 (2H, s), 6.99-7.04 (8H, m), 2.47-2.58 (8H, m), 1.49-1.61 (8H, m), 1.18-1.37 (40H, m), 0.82-0.91 (12H, m).

Polymer 2

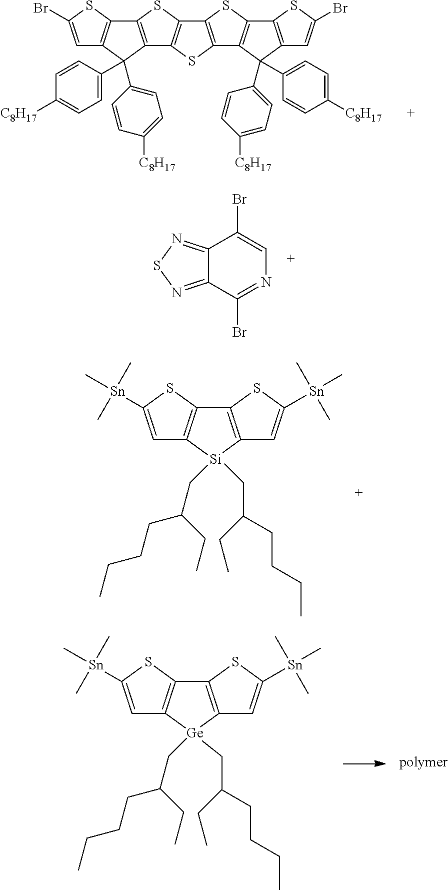

To a mixture of compound 5 (137.6 mg, 0.106 mmol), 4,7-dibromo-[1,2,5]thiadiazolo[3,4-c]pyridine (125.3 mg, 0.425 mmol), 7,7-bis-(2-ethyl-hexyl)-2,5-bis-trimethylstannanyl-7H-3,4-dithia-7-sila-cyclopenta[a]pentalene (208.5 mg, 0.280 mmol), 7,7-bis-(2-ethyl-hexyl)-2,5-bis-trimethylstannanyl-7H-3,4-dithia-7-germa-cyclopenta[a]pentalene (197.8 mg, 0.251 mmol), tri-o-tolyl phosphine (28 mg, 0.09 mmol) and tris(dibenzylideneacetone)dipalladium(0) (17.7 mg, 0.03 mmol) was added degassed anhydrous toluene (10 cm³) and the reaction mixture further degassed by bubbling nitrogen for 20 minutes. The mixture heated at 110° C. in a pre-heated block for 17 hours. Bromo-benzene (0.03 cm³) added and the mixture stirred at 110° C. for 30 minutes followed by addition of phenyltributyltin (0.14 cm³). The reaction mixture heated at 110° C. for a further 30 minutes. The reaction mixture allowed to cool slightly and poured into stirred methanol (100 cm³). The solid collected by filtration and subjected to Soxhlet extraction: acetone, 40-60 petrol, 80-100 petrol and cyclohexanes. The cyclohexanes extract was concentrated in vacuo and poured into stirred 2-propanol (200 cm³) and the polymer precipitate collected by filtration to give polymer 2 (148 mg, 36%) as a black solid. GPC (chlorobenzene, 50° C.) Mₙ=21,000 g/mol, M_{w}=47,000 g/mol.

### Example 3

Compound 6

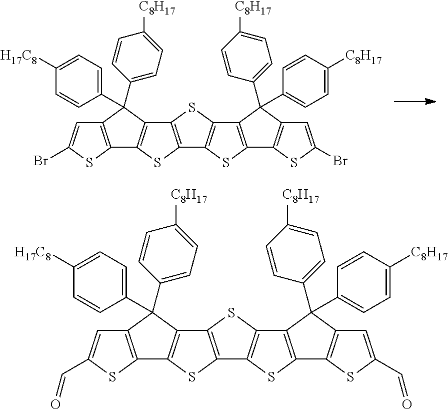

To a solution of compound 5 (245 mg, 0.19 mmol) in anhydrous tetrahydrofuran (9 cm³) at −78° C. was added dropwise n-butyllithium (0.25 cm³, 0.62 mmol, 2.5 M in hexane) over 20 minutes. After addition, the reaction mixture was stirred at −78° C. for 70 minutes before N,N-dimethylformamide (0.38 cm³, 4.9 mmol) was added. The mixture was then allowed to warm to 23° C. with stirring over 65 hours. Dichloromethane (30 cm³) and water (60 cm³) were added and the organic layer extracted, additionally washing the aqueous layer with dichloromethane (20 cm³). The combined organic extracts are then washed with water (50 cm³) and brine (75 cm³) diluted with water (25 cm³), extracting the aqueous layer each time with additional dichloromethane (25 cm³). The combined organic extracts are dried over anhydrous magnesium sulfate, filtered and the solvent removed in vacuo. The crude was partially purified by column chromatography using a graded solvent system (40-60 petrol:dichloromethane; 1:0-1:4) to give compound 6 (68 mg, 30%) as a partially pure yellow/orange solid. 1H NMR (400 MHz, CDCl₃) 9.82 (2H, s), 7.68 (2H, s), 7.09-7.14 (8H, m), 7.03-7.07 (8H, m), 2.54 (8H, t, J 7.9), 1.52-1.61 (8H, m), 1.21-1.36 (40H, m), 0.86-0.89 (12H, m).

Compound 7

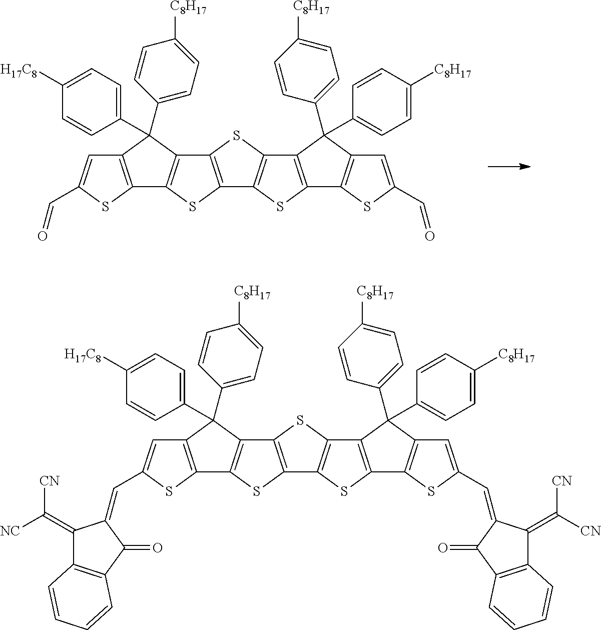

To a solution of partially purified compound 6 (68 mg, 0.06 mmol) in anhydrous chloroform (6 cm³) was added pyridine (0.32 cm³, 4.0 mmol). The mixture was then degassed with nitrogen before 3-(dicyanomethylidene)indan-1-one (77 mg, 0.40 mmol) was added. The solution was then further degassed for 10 minutes and stirred at 23° C. for 2.5 hours. The reaction mixture was then added to methanol (100 cm³), washing in with methanol (2×10 cm³) and dichloromethane (5 cm³). The suspension was stirred for 10 minutes before the precipitated solid was collected by vacuum filtration and washed with methanol (3×10 cm³). The crude product was then partially purified by column chromatography using a graded solvent system (40-60 petrol:dichloromethane; 1:0-2:3). A portion of the partially purified material is then further purified by preparative TLC (40-60 petrol:dichloromethane; 2:3) to give compound 7 as a blue solid. 1H NMR (400 MHz, CD₂Cl₂) 8.83 (2H, s), 8.63-8.67 (2H, m), 7.88-7.93 (2H, m), 7.70-7.82 (6H, m), 6.92-7.29 (16H, m), 2.56 (8H, t, J 7.8), 0.60-1.84 (60H, m).

### Example 4—Transistor Fabrication and Measurement

Top-gate thin-film organic field-effect transistors (OFETs) were fabricated on glass substrates with photolithographically defined Au source-drain electrodes. A 7 mg/cm³ solution of the organic semiconductor in dichlorobenzene was spin-coated on top (an optional annealing of the film is carried out at 100° C., 150° C. or 200° C. for between 1 and 5 minutes) followed by a spin-coated fluoropolymer dielectric material (Lisicon® D139 from Merck, Germany). Finally a photolithographically defined Au gate electrode was deposited. The electrical characterization of the transistor devices was carried out in ambient air atmosphere using computer controlled Agilent 4155C Semiconductor Parameter Analyser. Charge carrier mobility in the saturation regime (μₛₐₜ) was calculated for the compound. Field-effect mobility was calculated in the saturation regime (V_{d}>(V_{g}−V₀)) using equation (1):

$${( \frac{{dI}_{d}^{sat}}{{dV}_{g}} )}_{{V}_{d}} = \frac{{WC}_{i}}{L} {μ}^{sat} ( {V}_{g} - {V}_{0} ) \qquad ( 1 )$$

where W is the channel width, L the channel length, Cᵢ the capacitance of insulating layer, V_{g} the gate voltage, V₀ the turn-on voltage, and μₛₐₜ is the charge carrier mobility in the saturation regime. Turn-on voltage (V₀) was determined as the onset of source-drain current.

The mobility (μₛₐₜ) for polymer 1 is 0.06 cm²/Vs.

### Example 5—Bulk Heterojunction Organic Photodetector Devices (OPDs)

Devices are fabricated onto glass substrates with six pre-patterned ITO dots of 5 mm diameter to provide the bottom electrode. The ITO substrates are cleaned using a standard process of ultrasonication in Decon90 solution (30 minutes) followed by washing with de-ionized water (×3) and ultrasonication in de-ionized water (30 minutes). The ZnO ETL layer was deposited by spin coating a ZnO nanoparticle dispersion onto the substrate and drying on a hotplate for 10 minutes at a temperature between 100 and 140° C. A formulation of polymer and [6,6]-phenyl-C₇₁-butyric acid methyl ester (PCBM[C70]) was prepared at a 1:1.5 or a 1:2 ratio in 1,2-dichlorobenzene at a concentration of 20 mg/ml, and stirred for 17 hours at 60° C. The formulation was then filtered through a 0.2 μm PTFE filter and the formulation used to coat the active layer. The active layer was deposited using blade coating (K101 Control Coater System from RK). The stage temperature was set to 70° C., the blade gap set between 2-15 μm and the speed set between 2-8 m/min targeting a final dry film thickness of 500 nm. Following coating the active layer was annealed at 100° C. for 10 minutes. The MoO₃ HTL layer was deposited by E-beam vacuum deposition from MoO₃ pellets at a rate of 1 Å/s, targeting 15 nm thickness. Finally, the top silver electrode was deposited by thermal evaporation through a shadow mask, to achieve Ag thickness between 40-80 nm.

The J-V curves are measured using a Keithley 4200 system under light and dark conditions at a bias from +5 to −5 V. The light source was a 580 nm LED with power 0.5 mW/cm².

The EQE of OPD devices are characterized between 400 and 1100 nm under −2V bias, using an External Quantum Efficiency (EQE) Measurement System from LOT-QuantumDesign Europe. The EQE value at 850 nm for Polymer 2 is 21%.

### Examples 6-8: Small Molecules with Terminal Withdrawing Groups

Molecular structures were optimized at B3LYP/6-31G* level using Firefly QC package (see Alex A. Granovsky, Firefly version 8, www http://classic.chem.msu.su/gran/firefly/index.html), which is partially based on the GAMESS (US) source code (see M. W. Schmidt, K. K. Baldridge, J. A. Boatz, S. T. Elbert, M. S. Gordon, J. H. Jensen, S. Koseki, N. Matsunaga, K. A. Nguyen, S. Su, T. L. Windus, M. Dupuis, J. A. Montgomery J. Comput. Chem. 14, 1347-1363 (1993)).

E_{HOMO} and E_{LUMO} are defined as the eigenvalues of, respectively, the highest occupied and lowest unoccupied Kohn-Sham molecular orbitals, and are used as approximations of, respectively, ionisation potential (IP) and electron affinity (EA). E_{g} is defined as |E_{LUMO}-E_{HOMO}| and is the transport band gap of the material. S⁰-S¹ is the vertical excitation energy from the ground state S⁰ to the first singlet excited state S¹, and is used as the measure of the optical band gap E_{g}(opt).

An approximate relation between E_{HOMO}, E_{LUMO} and E_{g} of donor and acceptor materials in a bulk-heterojunction is known as the Scharber model [M. C. Scharber, D. Mühlbacher, M. Koppe, P. Denk, C. Waldauf, A. J. Heeger, C. J. Brabec, Adv. Mater. 2006, 18, 789-794]. It is widely accepted that when the donor material of the donor-acceptor blend absorbs light and forms an excited state, the excited electron must hop onto the neighbouring acceptor site in order for the free carriers to be formed. The driving force of this process is the energetic difference between the excited state of the donor material and the electron affinity (approximated by E_{LUMO}) of the acceptor material and has been empirically found to be at least ca. 0.35 eV for charge generation to be efficient [D. Veldman, S. C. J. Meskers, R. A. J. Janssen, Adv. Funct. Mater. 2009, 19, 1939-1948; M. C. Scharber, N. S. Sariciftci, Progr. Polym. Sci. 38 (2013) 1929-1940]. Therefore, tuning of acceptor's E_{LUMO} is of paramount importance, lowering its value will increase the driving force for charge generation and may allow using lower-bandgap donor material, whilst increasing E_{LUMO} may hinder charge generation. For the present OSC materials, owing to their small optical band gap, another mechanism is also possible: light absorption by the acceptor followed by hole injection to the donor material, driven by the energy difference between E_{HOMO} of donor and acceptor, respectively [W. Zhao, D. Qian, S. Zhang, S. Li, O. Inganäs, F. Gao, J. Hou, Adv. Mater. 2016, DOI: 10.1002/adma.201600281]. This mechanism is responsible for non-negligible external quantum efficiency beyond the absorption edge of the donor material, and retaining of this advantage of the acceptor material requires careful tuning of HOMO energy.

### Comparative Example 1

Compound C1 as shown below is calculated as a reference.

| | | E_{HOMO}/ | E_{LUMO}/ | E_{g}/ | S⁰-S¹/ |
|---|---|---|---|---|---|
| No. | Structure | eV | eV | eV | eV |
| C1 | 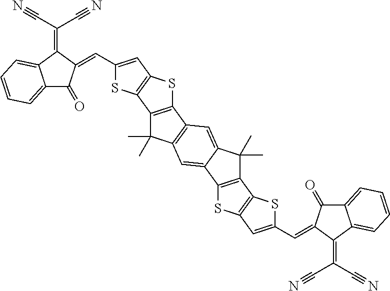 [SMILES: CC1(C)c2cc3c(cc2-c2sc4cc(/C=C5\C(=O)c6ccccc6C5=C(C#N)C#N)sc4c21)C(C)(C)c1c-3sc2cc(/C=C3\C(=O)c4ccccc4C3=C(C#N)C#N)sc12] | −5.54 | −3.41 | 2.13 | 1.86 |

### Examples 6-8

The computed values of E_{HOMO}, E_{LUMO}, E_{g} and S⁰-S¹ of compound C1 (whilst being different from experimentally determined IP, EA and E_{g}) are compared with the computed values of compounds 6-8 of formula VI.

| | | E_{HOMO}/ | E_{LUMO}/ | E_{g}/ | S⁰-S¹/ |
|---|---|---|---|---|---|
| No. | Structure | eV | eV | eV | eV |
| 6 | 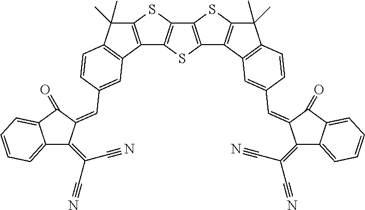 [SMILES: CC1(C)c2ccc(/C=C3\C(=O)c4ccccc4C3=C(C#N)C#N)cc2-c2c1sc1c2sc2c3c(sc21)C(C)(C)c1ccc(/C=C2\C(=O)c4ccccc4C2=C(C#N)C#N)cc1-3] | −5.46 | −3.31 | 2.15 | 1.86 |
| 7 | 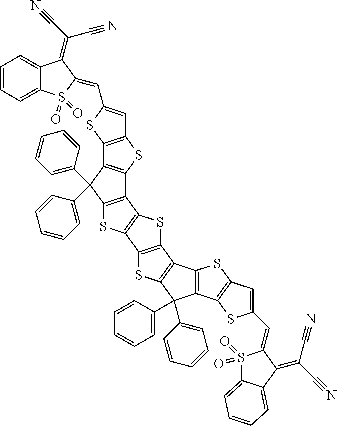 [SMILES: N#CC(C#N)=C1/C(=C/c2cc3sc4c(c3s2)C(c2ccccc2)(c2ccccc2)c2sc3c(sc5c6c(sc53)C(c3ccccc3)(c3ccccc3)c3c-6sc5cc(/C=C6/C(=C(C#N)C#N)c7ccccc7S6(=O)=O)sc35)c2-4)S(=O)(=O)c2ccccc21] | −5.58 | −3.40 | 2.18 | 1.88 |
| 8 | 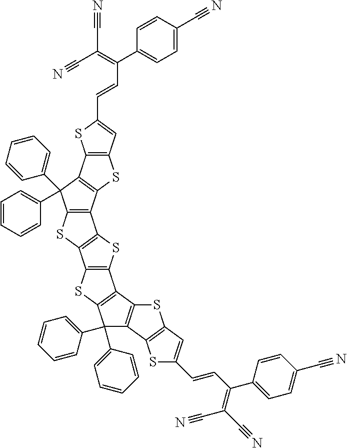 [SMILES: N#CC(C#N)=C(/C=C/c1cc2sc3c(c2s1)C(c1ccccc1)(c1ccccc1)c1sc2c(sc4c5c(sc42)C(c2ccccc2)(c2ccccc2)c2c-5sc4cc(/C=C/C(=C(C#N)C#N)c5ccc(C#N)cc5)sc24)c1-3)c1ccc(C#N)cc1] | −5.61 | −3.37 | 2.24 | 1.80 |

The E_{LUMO} of compounds 6-8 are found to be close or slightly lower to that of compound C1, indicating a similar or slightly stronger electron affinity. Calculated band gaps of compounds 6-8 are similar or slightly smaller than that of C1.

1. A compound comprising one or more divalent heteroarylene units of formula I 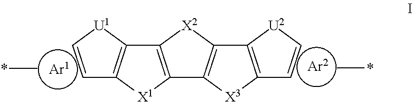
wherein the individual radicals, independently of each other and on each occurrence identically or differently, have the following meanings
Ar^{1,2} arylene or heteroarylene that has from 5 to 20 ring atoms, is mono- or polycyclic, optionally contains fused rings, and is unsubstituted or substituted by one or more identical or different groups L,
X¹, X², X³ O, S or Se,
U¹ CR¹R², SiR¹R², GeR¹R², NR′ or C═O,
U² CR³R⁴, SiR³R⁴, GeR³R⁴, NR³ or C═O,
R¹⁻⁴ H, F, Cl or straight-chain, branched or cyclic alkyl with 1 to 30 C atoms, in which one or more CH₂ groups are optionally replaced by —O—, —S—, —C(═O)—, —C(═S)—, —C(═O)—O—, —O—C(═O)—, —NR⁰—, —SiR⁰R⁰⁰—, —CF₂—, —CR⁰═CR⁰⁰—, —CY¹═CY²— or —C≡C— in such a manner that 0 and/or S atoms are not linked directly to one another, and in which one or more H atoms are optionally replaced by F, Cl, Br, I or CN, and in which one or more CH₂ or CH₃ groups are optionally replaced by a cationic or anionic group, or aryl, heteroaryl, arylalkyl, heteroarylalkyl, aryloxy or heteroaryloxy, wherein each of the aforementioned cyclic groups has 5 to 20 ring atoms, is mono- or polycyclic, does optionally contain fused rings, and is unsubstituted or substituted by one or more identical or different groups L,
and the pair of R¹ and R² and/or the pair of R³ and R⁴ together with the C, Si or Ge atom to which they are attached, may also form a spiro group with 5 to 20 ring atoms which is mono- or polycyclic, does optionally contain fused rings, and is unsubstituted or substituted by one or more identical or different groups L,
L F, Cl, —NO₂, —CN, —NC, —NCO, —NCS, —OCN, —SCN, R⁰, OR⁰, SR⁰, —C(═O)X⁰, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO₂R⁰, —OH, —NO₂, —CF₃, —SF₅, or optionally substituted silyl, or carbyl or hydrocarbyl with 1 to 30 C atoms that is optionally substituted and optionally comprises one or more hetero atoms,
R⁰, R⁰⁰ H or straight-chain or branched alkyl with 1 to 20 C atoms that is optionally fluorinated,
X⁰ halogen.

2. The compound of claim 1, wherein Ar¹ and Ar² are selected from the following formulae 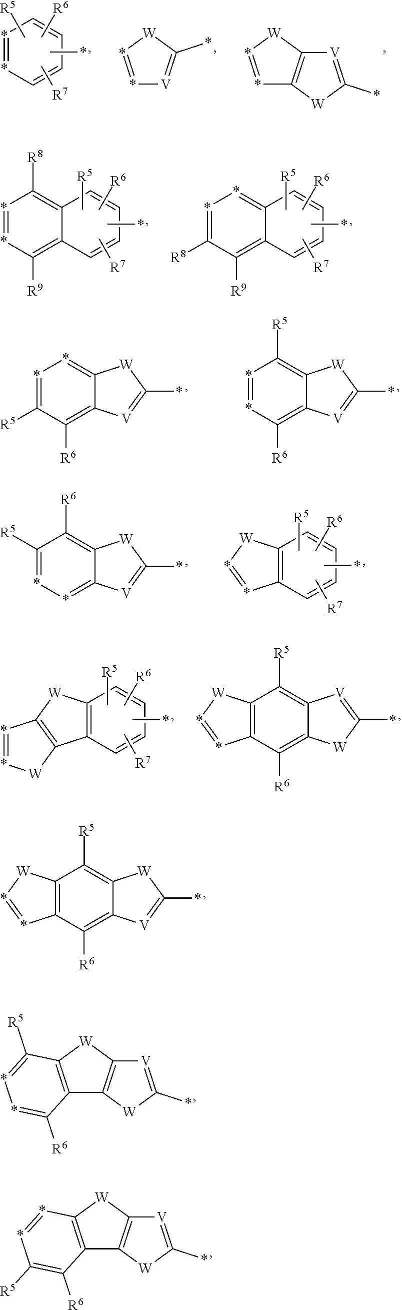 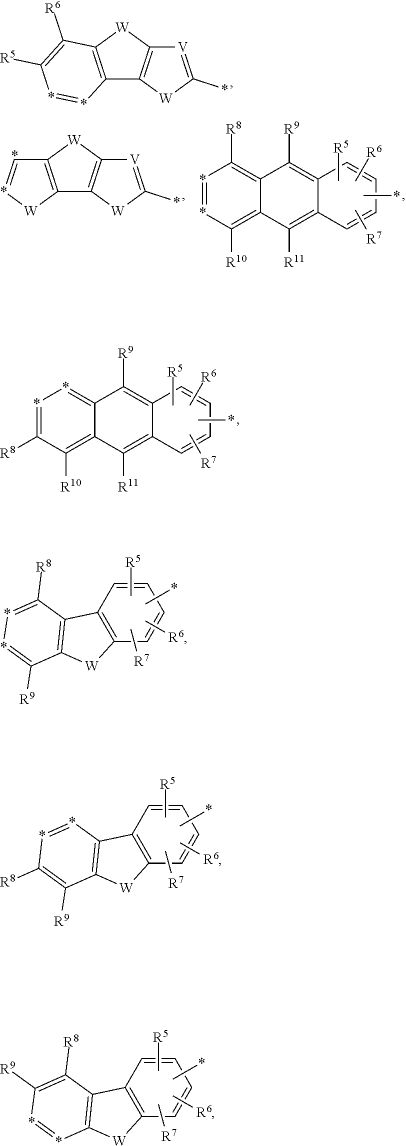
wherein the individual radicals, independently of each other and on each occurrence identically or differently, have the following meanings
W S, O or Se,
V CR¹ or N,
R⁵⁻¹¹ H, F, Cl or straight-chain, branched or cyclic alkyl with 1 to 30 C atoms, in which one or more CH₂ groups are optionally replaced by —O—, —S—, —C(═O)—, —C(═S)—, —C(═O)—O—, —O—C(═O)—, —NR⁰—, —SiR⁰R⁰⁰—, —CF₂—, —CR⁰═CR⁰⁰—, —CY¹═CY²— or —C≡C— in such a manner that O and/or S atoms are not linked directly to one another, and in which one or more H atoms are optionally replaced by F, Cl, Br, I or CN, and in which one or more CH₂ or CH₃ groups are optionally replaced by a cationic or anionic group, or aryl, heteroaryl, arylalkyl, heteroarylalkyl, aryloxy or heteroaryloxy, wherein each of the aforementioned cyclic groups has 5 to 20 ring atoms, is mono- or polycyclic, does optionally contain fused rings, and is unsubstituted or substituted by one or more identical or different groups L as defined in claim 1.

3. The compound of claim 2, wherein Ar¹ and Ar² are selected from the following formulae 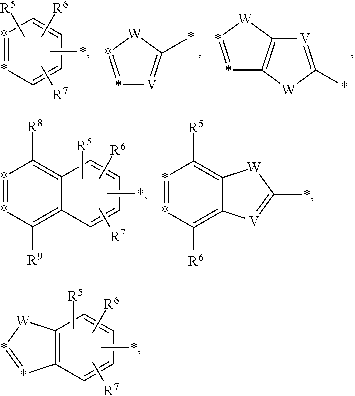
wherein W, V and R⁵⁻⁹ have the meanings given in claim 2.

4. The compound according to claim 1, wherein the units of formula I are selected from the following subformulae 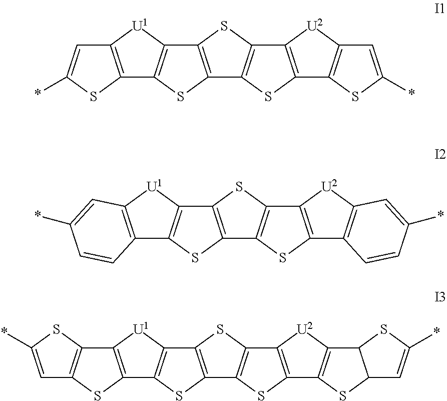
wherein U^{1,2} are as defined in claim 1.

5. The compound according to claim 2, wherein R¹⁻¹¹ are selected from the following groups
the group consisting of R, —OR and —SR wherein R is straight-chain or branched alkyl with 1 to 20 C atoms which is unsubstituted or substituted by one or more F atoms,
the group consisting of —C(═O)—R, —C(═O)—OR, —OC(═O)—R, —C(═O)—NHR and —C(═O)—NRRⁿ, wherein R and Rⁿ are independently of each other straight-chain or branched alkyl with 1 to 20 C atoms that is optionally fluorinated, and
the group consisting of aryl, aryloxy, heteroaryl and heteroaryloxy, each of which has 4 to 20 ring atoms and optionally contains fused rings and is unsubstituted or substituted by one or more groups L.

6. The compound according to claim 1, which is a conjugated polymer comprising one or more units of formula I, and further comprising one or more arylene or heteroarylene units that have from 5 to 20 ring atoms, are mono- or polycyclic, do optionally contain fused rings, are unsubstituted or substituted by one or more identical or different groups L, and are either selected of formula I or are structurally different from formula I, and wherein all the aforementioned units are directly connected to each other.

7. The compound of claim 6, which comprises one or more repeating units of formula II1 or II2, and optionally one or more repeating units of formula II3:
—(Ar¹)ₐ—U—(Ar²)_{b}—(Ar³)_{c}—(Ar⁴)_{d}— II1
—(Ar¹)ₐ—(Ar²)_{b}—U—(Ar³)_{c}—(Ar⁴)_{d}— II2
—(Ar¹)ₐ—(Ar²)_{b}—(Ar³)_{c}—(Ar⁴)_{d}— II3
wherein the individual radicals, independently of each other and on each occurrence identically or differently, have the following meanings
U a unit of formula I,
Ar¹⁻⁴ arylene or heteroarylene that has 5 to 20 ring atoms, is mono- or polycyclic, does optionally contain fused rings, is unsubstituted or substituted by one or more identical or different groups L, and is different from U, and
a, b, c, d 0 or 1, wherein in formula II3 a+b+c+d≥1.

8. The compound according to claim 7, which is selected of formula III: 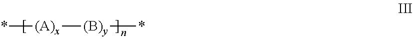
wherein
A is a unit of formula I, II1 or II2,
B is a unit of formula I, II1, II2 or II3,
x is >0 and ≤1,
y is ≥0 and <1,
x+y is 1, and
n is an integer ≥5.

9. The compound according to claim 8, which is selected from the following formulae 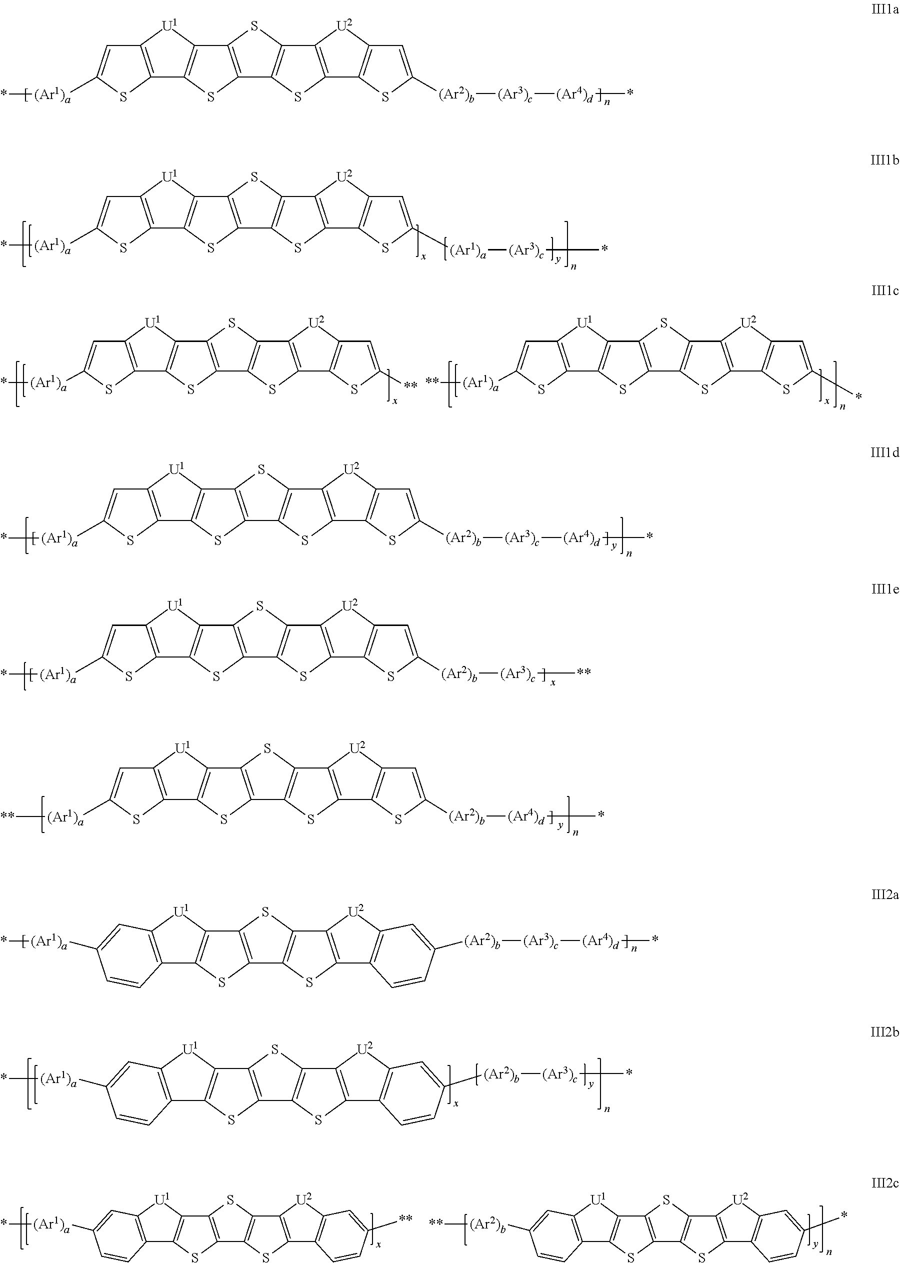 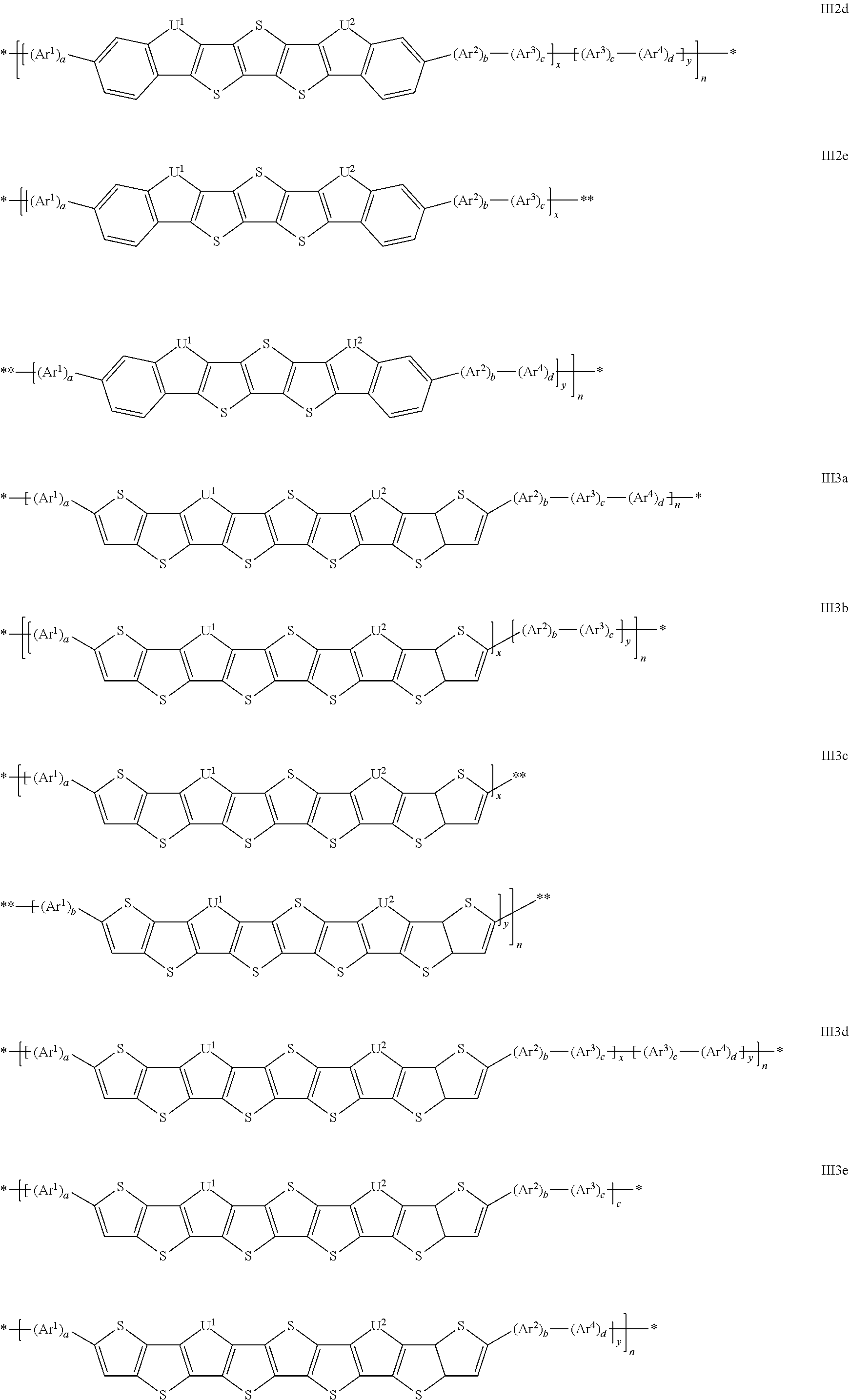
wherein
U¹ is CR¹R², SiR¹R², GeR¹R², NR¹ or C═O,
U² is CR³R⁴, SiR³R⁴, GeR³R⁴, NR³ or C═O,
Ar¹⁻⁴ is arylene or heteroarylene that has 5 to 20 ring atoms, is mono- or polycyclic, does optionally contain fused rings, is unsubstituted or substituted by one or more identical or different groups L,
a-d are each 0 or 1,
x is >0 and ≤1,
y is ≥0 and <1,
x+y is 1, and
n is an integer ≥5.

10. The compound according to claim 8, which is selected of formula IV
R²¹-chain-R²³ IV
wherein
“chain” denotes a polymer chain selected from formulae III,
R²¹ and R²³ denote, independently of each other, F, Cl, —NO₂, —CN, —NC, —NCO, —NCS, —OCN, —SCN, R⁰, OR⁰, SR⁰, —C(═O)X⁰, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO₂R⁰, —OH, —NO₂, —CF₃, —SF₅, or optionally substituted silyl, or carbyl or hydrocarbyl with 1 to 30 C atoms that is optionally substituted and optionally comprises one or more hetero atoms,
or denote, independently of each other, H, F, Br, Cl, I, —CH₂Cl, —CHO, —CR′═CR″₂, —SiR′R″R′″, —SiR′X′X″, —SiR′R″X′, —SnR′R″R′″, —BR′R″, —B(OR′)(OR″), —B(OH)₂, —O—SO₂—R′, —C≡CH, —C≡C—SiR′₃, —ZnX′ or an endcap group,
X′ and X″ denote halogen, and
R′, R″ and R′″ denote, independently of each other, H or straight-chain or branched alkyl with 1 to 20 C atoms that is optionally fluorinated, and
two of R′, R″ and R′″ may also form a cyclosilyl, cyclostannyl, cycloborane or cycloboronate group with 2 to 20 C atoms together with the respective hetero atom to which they are attached.

11. The compound according to claim 7, wherein one or more of Ar¹, Ar², Ar³ and Ar⁴ denote arylene or heteroarylene of the following formulae 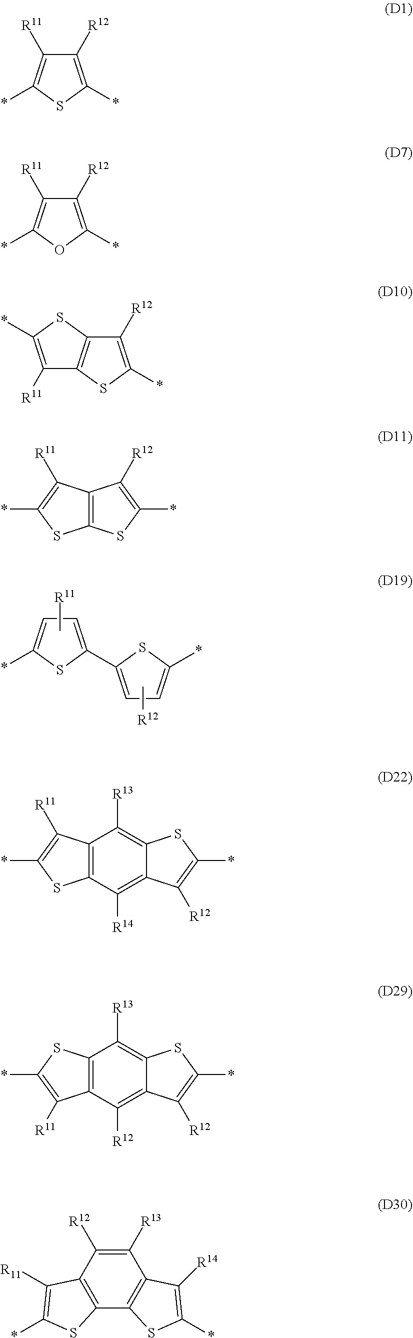 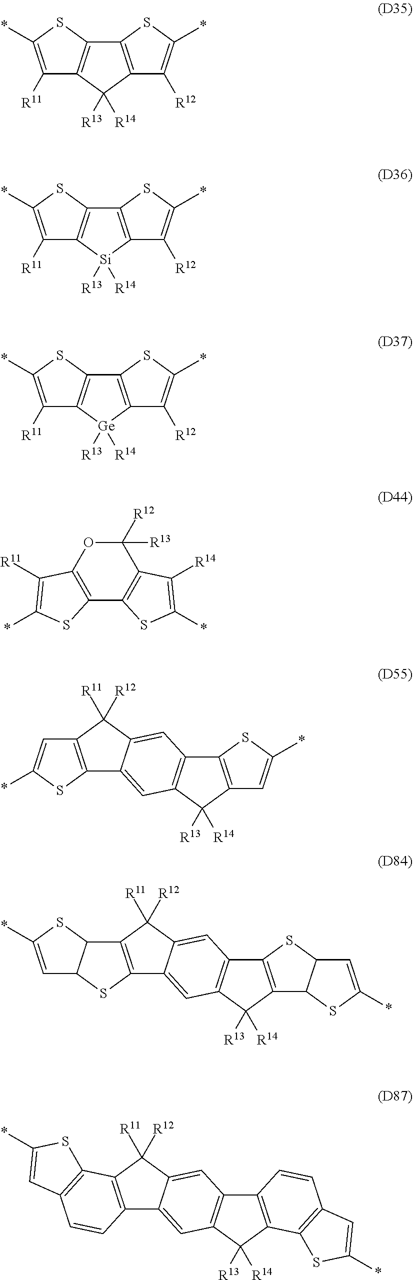 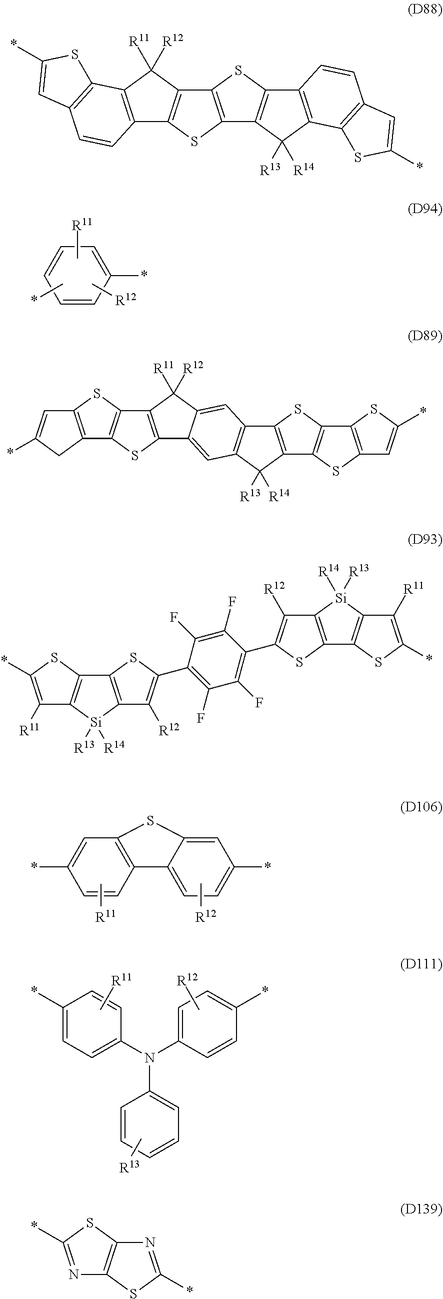 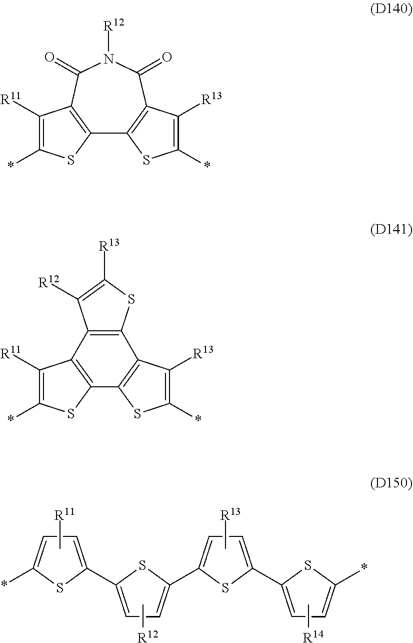
wherein
R¹¹, R¹², R¹³, and R¹⁴, independently of each other, denote H, F, Cl, —NO₂, —CN, —NC, —NCO, —NCS, —OCN, —SCN, R⁰, OR⁰, SR⁰, —C(═O)X⁰, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO₂R⁰, —OH, —NO₂, —CF₃, —SF₅, or optionally substituted silyl, or carbyl or hydrocarbyl with 1 to 30 C atoms that is optionally substituted and optionally comprises one or more hetero atoms,
R⁰, R⁰⁰ H or straight-chain or branched alkyl with 1 to 20 C atoms that is optionally fluorinated,
X⁰ halogen.

12. The compound according to claim 7, wherein one or more of Ar¹, Ar², Ar³ and Ar⁴ denote arylene or heteroarylene of the following formulae 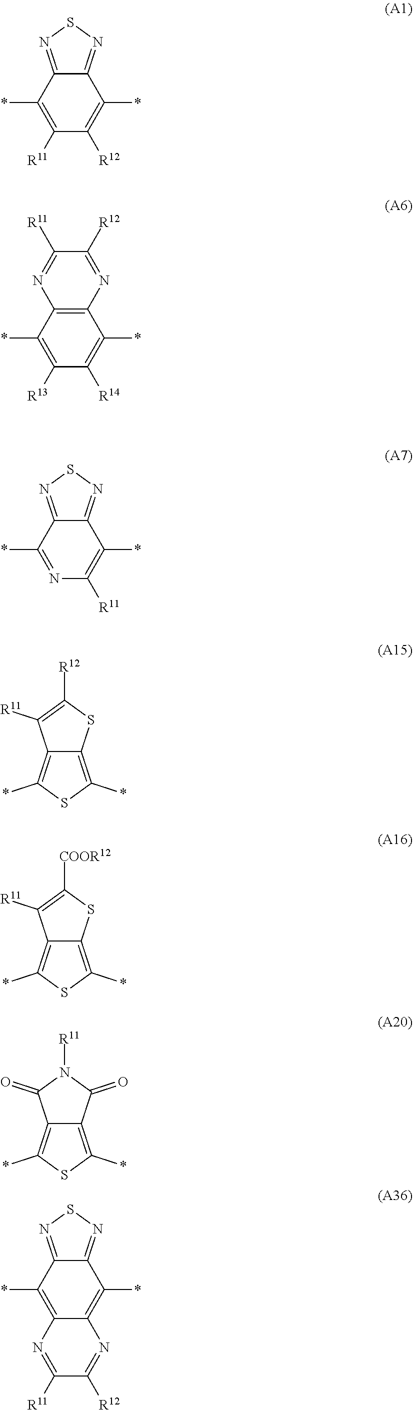 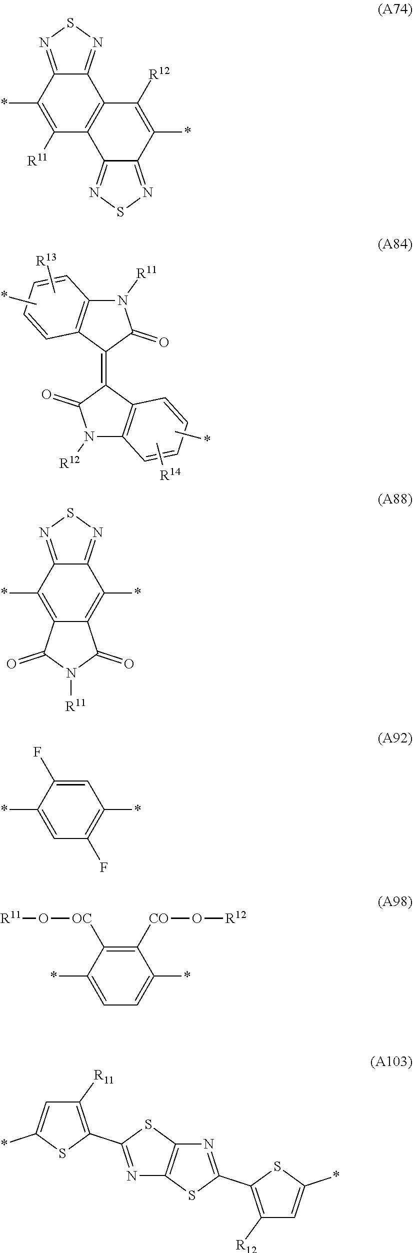
wherein
R¹¹, R¹², R¹³, and R¹⁴, independently of each other, denote H, F, Cl, —NO₂, —CN, —NC, —NCO, —NCS, —OCN, —SON, R⁰, OR⁰, SR⁰, —C(═O)X⁰, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO₂R⁰, —OH, —NO₂, —CF₃, —SF₅, or optionally substituted silyl, or carbyl or hydrocarbyl with 1 to 30 C atoms that is optionally substituted and optionally comprises one or more hetero atoms,
R⁰, R⁰⁰ H or straight-chain or branched alkyl with 1 to 20 C atoms that is optionally fluorinated,
X⁰ halogen.

13. The compound according to claim 7, wherein one or more of Ar¹, Ar², Ar² and Ar⁴ denote arylene or heteroarylene of the following formulae 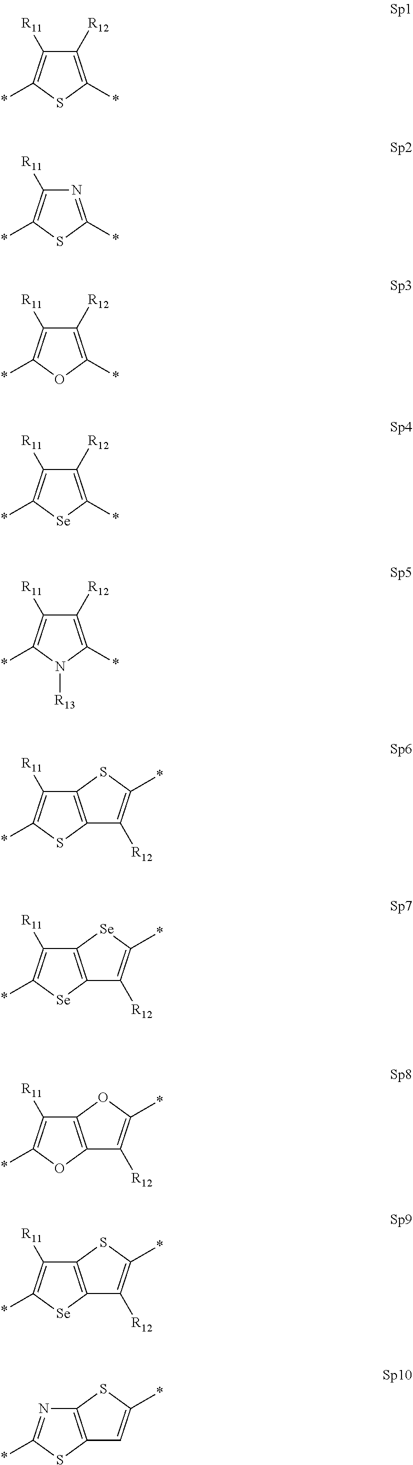 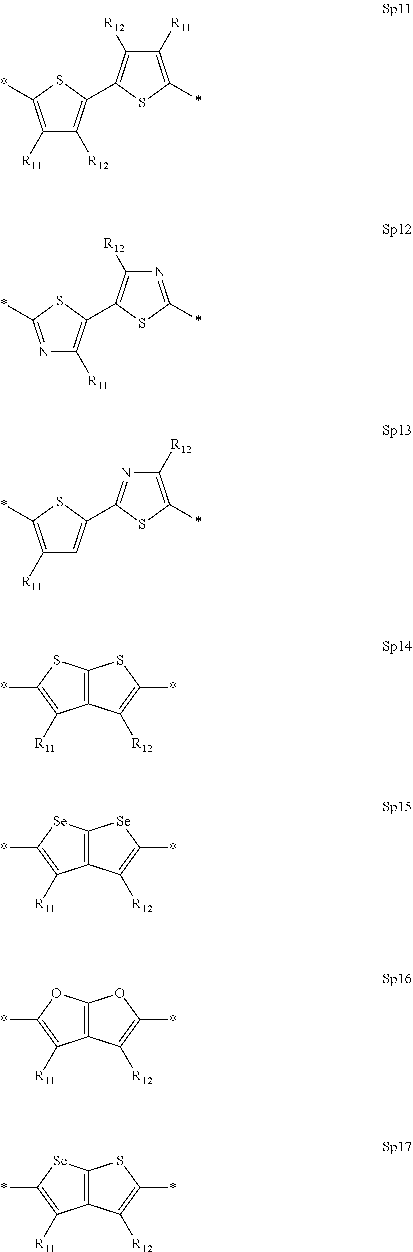
wherein
R¹¹ and R¹², independently of each other, denote H, F, Cl, —NO₂, —CN, —NC, —NCO, —NCS, —OCN, —SCN, R⁰, OR⁰, SR⁰, —C(═O)X⁰, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO₂R⁰, —OH, —NO₂, —CF₃, —SF₅, or optionally substituted silyl, or carbyl or hydrocarbyl with 1 to 30 C atoms that is optionally substituted and optionally comprises one or more hetero atoms,
R⁰, R⁰⁰ H or straight-chain or branched alkyl with 1 to 20 C atoms that is optionally fluorinated,
X⁰ halogen.

14. (canceled)

15. A compound of formula V1 or V2
R²³—(Ar¹)ₐ—U—(Ar²)_{b}—(Ar³)_{c}(Ar⁴)_{d}—R²⁴ V1
R²³—(Ar¹)ₐ—(Ar²)_{b}—U—(Ar³)_{c}(Ar⁴)_{d}—R²⁴ V2
wherein
U, Ar¹⁻⁴, a, b, c and d have the meanings given in claim 7,
R²³ and R²⁴ are independently of each other H, Cl, Br, I, O-tosylate, O-triflate, O-mesylate, O-nonaflate, —SiMe₂F, —SiMeF₂, —O—SO₂Z¹, —B(OZ²)₂, —CZ³═C(Z³)₂, —C≡CH, —C≡CSi(Z¹)₃, —ZnX⁰ or —Sn(Z⁴)₃,
X⁰ is halogen, and
Z¹⁻⁴ are each independently C₁₋₁₀ alkyl or C₆₋₁₂ aryl, each being optionally substituted, and two groups Z² may also form a cycloboronate group having 2 to 20 C atoms together with the B- and O-atoms,
wherein at least one of R²³ and R²⁴ is different from H.

16. A compound of formula VI
R^{T1}—(Ar¹)ₑ—(Ar²)_{f}—[(Ar³)_{g}—(Ar⁴)ₕ—U—(Ar⁵)ᵢ—(Ar⁶)ₖ]ₒ—(Ar⁷)ₗ—(Ar⁸)ₘ—R^{T2} VI
wherein the individual radicals, independently of each other and on each occurrence identically or differently, have the following meanings
U a unit of formula I as defined in claim 1,
Ar¹⁻⁸ arylene or heteroarylene that has from 5 to 20 ring atoms, is mono- or polycyclic, optionally contains fused rings, and is unsubstituted or substituted by one or more identical or different groups L as defined in claim 1, or —CY¹═CY²— or C≡C—,
Y¹, Y² H, F, Cl or CN,
R^{T1}, R^{T2} a carbyl or hydrocarbyl group with 1 to 30 C atoms that is optionally substituted by one or more groups L as defined in claim 1 and optionally comprises one or more hetero atoms,
e-m 0 or 1, and
o 1, 2 or 3.

17. The compound of claim 16, wherein R^{T1} and R^{T2} are selected from H, F, Cl, Br, —NO₂, —CN, —CF₃, R*, —CF₂—R*, —O—R*, —S—R*, —SO₂—R*, —SO₃—R*, —C(═O)—H, —C(═O)—R*, —C(═S)—R*, —C(═O)—CF₂—R*, —C(═O)—OR*, —C(═S)—OR*, —O—C(═O)—R*, —O—C(═S)—R*, —C(═O)—SR*, —S—C(═O)—R*, —C(═O)NR*R**, —NR*—C(═O)—R*, —NHR*, —NR*R**, —CR*═CR*R**, —C═C—R*, —C═C—SiR*R**R***, —SiR*R**R***, —CH═CH(CN), —CH═C(CN)₂, —C(CN)═C(CN)₂, —CH═C(CN)(R^{a}), CH═C(CN)—C(═O)—OR*, —CH═C(CO—OR*)₂, —CH═C(CO—NR*R**)₂, and the following formulae 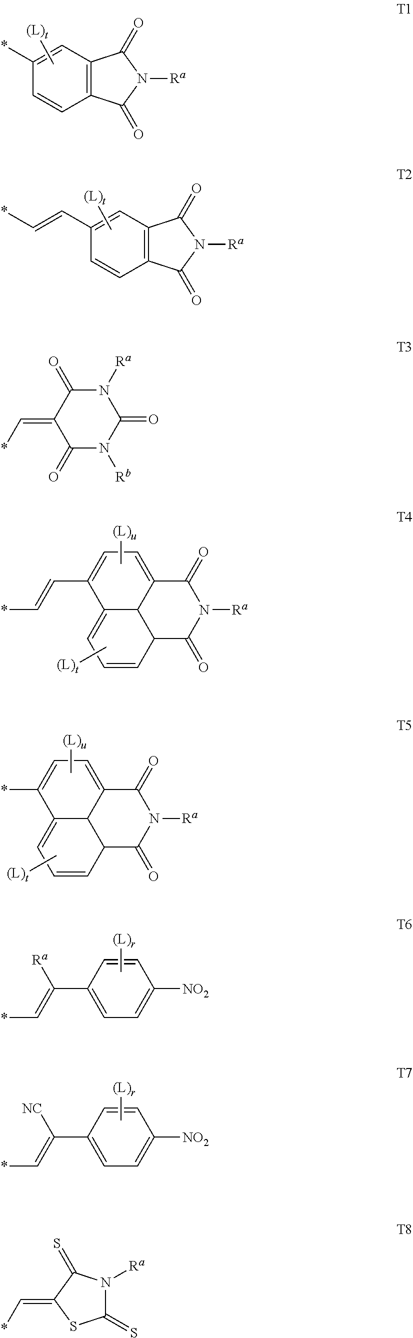 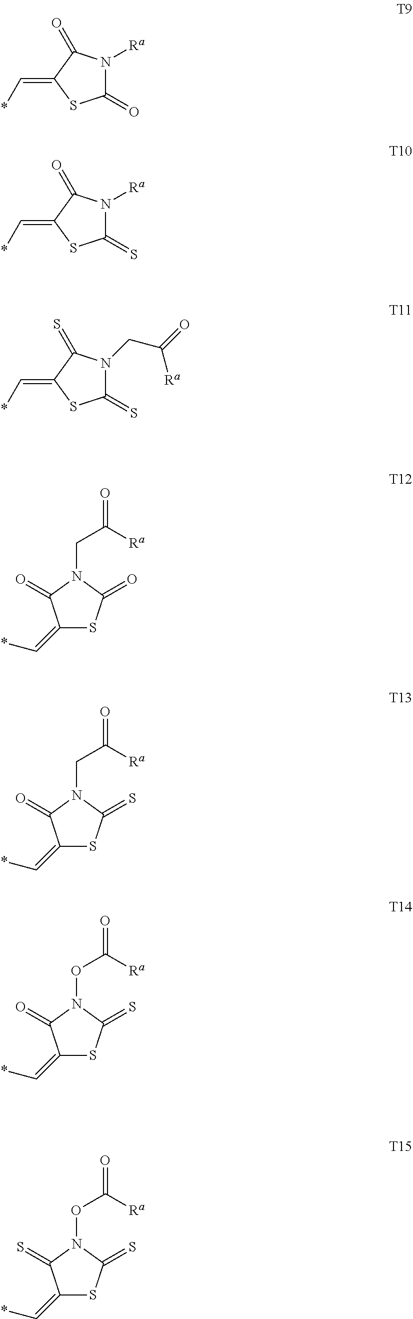 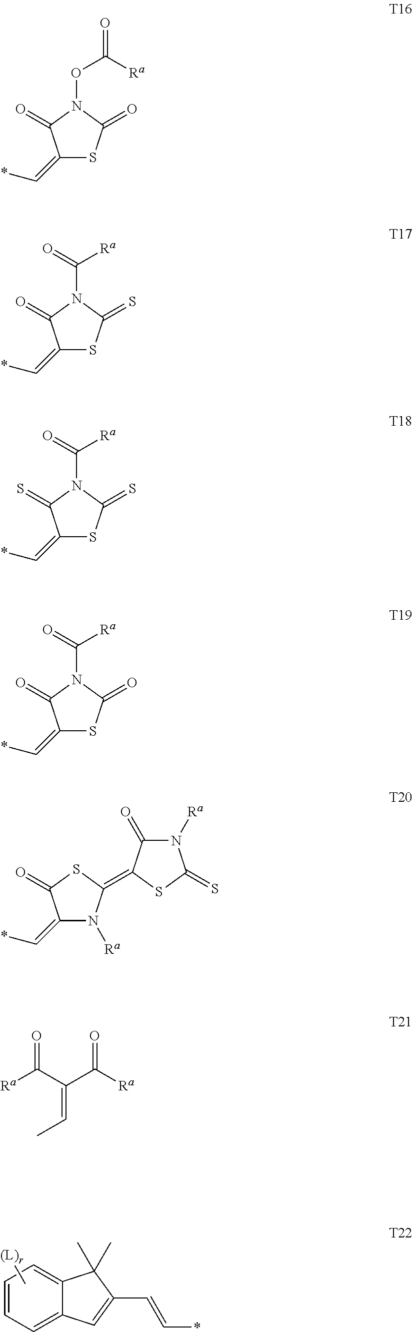 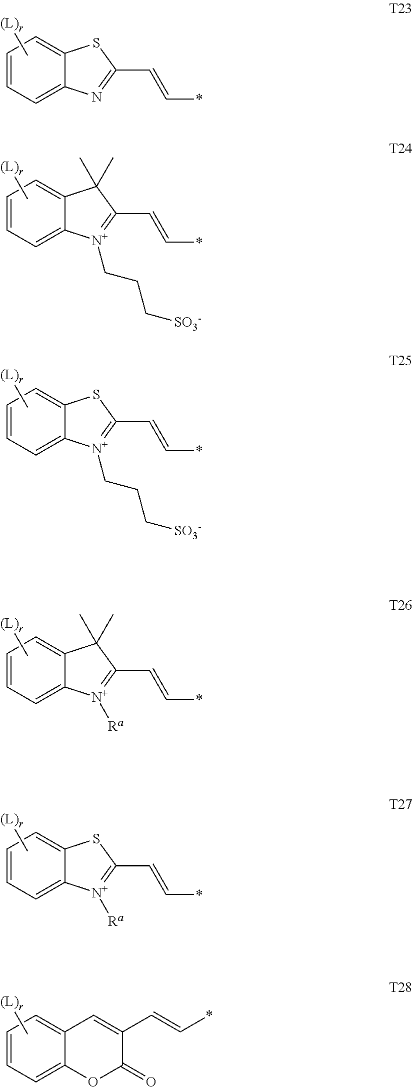 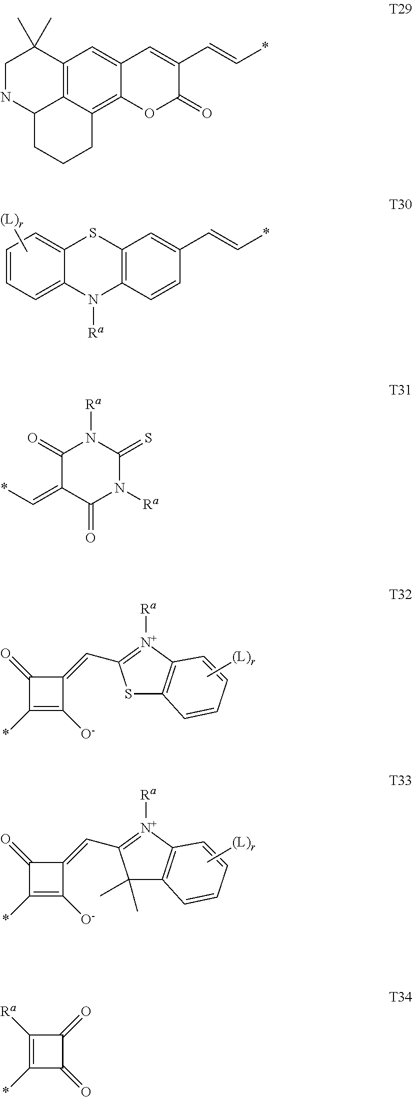 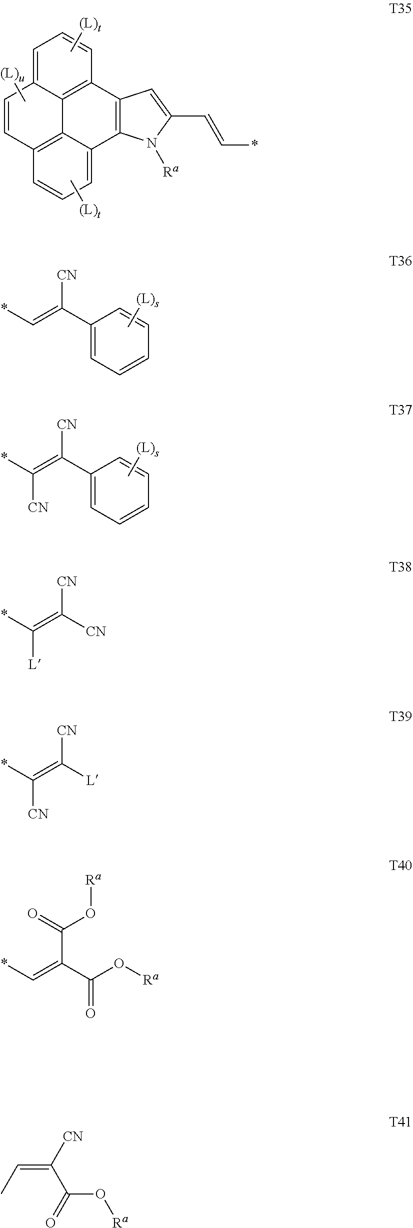 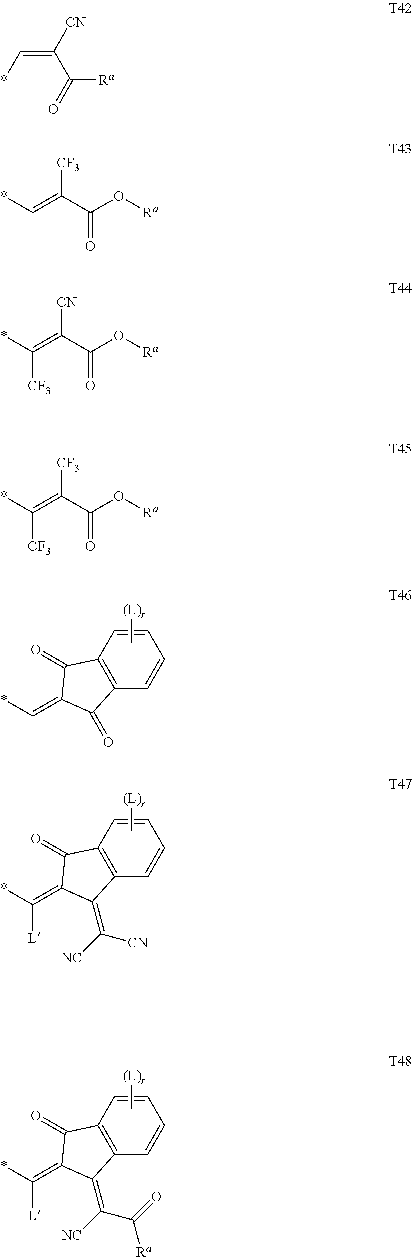 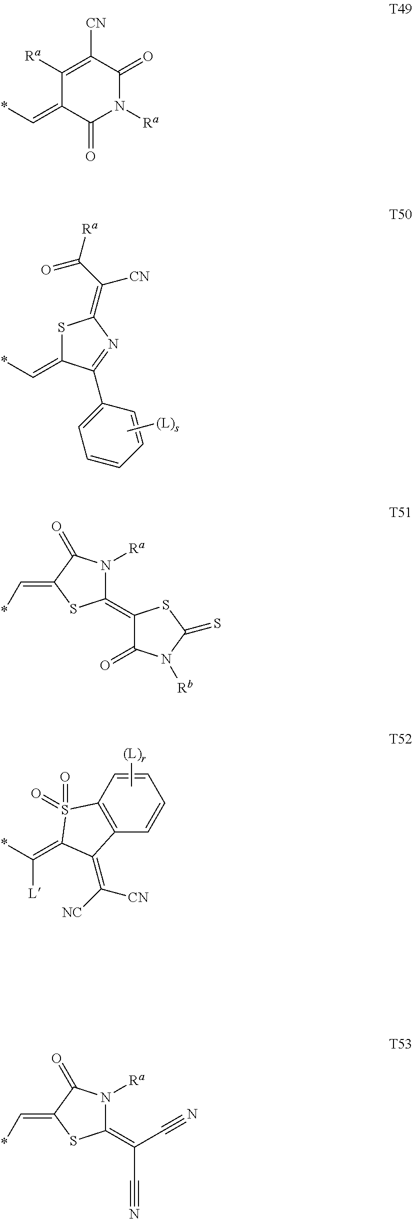
wherein the individual radicals, independently of each other and on each occurrence identically or differently, have the following meanings
R^{a}, R^{b} aryl or heteroaryl, each having from 4 to 30 ring atoms, optionally containing fused rings and being unsubstituted or substituted with one or more groups L, or one of the meanings given for L,
R*, R**, R*** alkyl with 1 to 20 C atoms which is straight-chain, branched or cyclic, and is unsubstituted, or substituted with one or more F or Cl atoms or CN groups, or perfluorinated, and in which one or more C atoms are optionally replaced by —O—, —S—, —C(═O)—, —C(═S)—, —SiR⁰R⁰⁰—, —NR⁰R⁰⁰—, —CHR⁰═CR⁰⁰— or —C≡C— such that O- and/or S-atoms are not directly linked to each other,
L F, Cl, —NO₂, —CN, —NC, —NCO, —NCS, —OCN, —SCN, R⁰, OR⁰, SR⁰, —C(═O)X⁰, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO₂R⁰, —OH, —NO₂, —CF₃, —SF₅, or optionally substituted silyl, or carbyl or hydrocarbyl with 1 to 30 C atoms that is optionally substituted and optionally comprises one or more hetero atoms,
L′ H or one of the meanings of L,
R⁰, R⁰⁰ H or straight-chain or branched alkyl with 1 to 12 C atoms that is optionally fluorinated,
Y¹, Y² H, F, Cl or CN,
X⁰ halogen,
r 0, 1, 2, 3 or 4,
s 0, 1, 2, 3, 4 or 5,
t 0, 1, 2 or 3, and
u 0, 1 or 2.

18. The compound of claim 16, wherein one or both of R^{T1} and R^{T2} denote an electron withdrawing group.

19. The compound according to claim 17, wherein R^{T1} and R^{T2} are selected from the following formulae

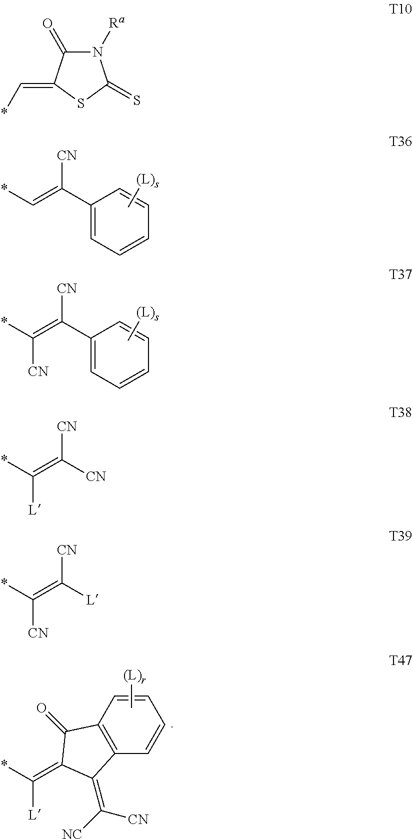

20. (canceled)

21. A composition comprising one or more compounds according to claim 1 and one or more additional compounds having one or more of semiconducting, charge transport, hole or electron transport, hole or electron blocking, electrically conducting, photoconducting or light emitting properties.

22. The composition of claim 21, wherein at least one of said one or more compounds is a p-type semiconductor, and said composition further comprises one or more n-type semiconductors.

23. The composition of claim 21, wherein at least one of said one or more compounds is an n-type semiconductor, and said composition further comprises one or more p-type semiconductors.

24. A bulk heterojunction (BHJ) formed from a composition according to claim 21.

25. A formulation comprising one or more compounds according to claim 1, and further comprising one or more solvents selected from organic solvents.

26. (canceled)

27. An electronic or optoelectronic device, or a component thereof, or an assembly comprising it, comprising a compound according to claim 1.

28. The electronic or optoelectronic device according to claim 27, which is selected from organic field effect transistors (OFET), organic thin film transistors (OTFT), organic light emitting diodes (OLED), organic light emitting transistors (OLET), organic photovoltaic devices (OPV), organic photodetectors (OPD), organic solar cells, dye-sensitized solar cells (DSSC), perovskite-based solar cells, laser diodes, Schottky diodes, photoconductors, photodetectors and thermoelectric devices.

29. The component according to claim 27, which is selected from charge injection layers, charge transport layers, interlayers, planarising layers, antistatic films, polymer electrolyte membranes (PEM), conducting substrates and conducting patterns.

30. The assembly according to claim 27, which is selected from integrated circuits (IC), radio frequency identification (RFID) tags, security markings, security devices, flat panel displays, backlights of flat panel displays, electrophotographic devices, electrophotographic recording devices, organic memory devices, sensor devices, biosensors and biochips.

31. A process of preparing a compound according to claim 6, said process comprising:
coupling one or more compounds of formula V1 or formula V2 with each other and/or with one or more monomers of formulae MI-MIV in an aryl-aryl coupling reaction
R²³—(Ar¹)ₐ—U—(Ar²)_{b}—(Ar³)_{c}—(Ar⁴)_{d}—R²⁴ V1
R²³—(Ar¹)ₐ—(Ar²)_{b}—U—(Ar³)_{c}—(Ar⁴)_{d}—R²⁴ V2
R²³—Ar¹—R²⁴ MI
R²³—Ar²—R²⁴ MII
R²³—Ar³—R²⁴ MIII
R²³—Ar⁴—R²⁴ MIV
wherein
U a unit of formula I,
Ar¹⁻⁴ arylene or heteroarylene that has 5 to 20 ring atoms, is mono- or polycyclic, does optionally contain fused rings, is unsubstituted or substituted by one or more identical or different groups L, and is different from U,
a, b, c, d 0 or 1, wherein in formula II3 a+b+c+d≥1,
R²³ and R²⁴ are independently of each other H, Cl, Br, I, O-tosylate, O-triflate, O-mesylate, O-nonaflate, —SiMe₂F, —SiMeF₂, —O—SO₂Z¹, —B(OZ²)₂, —CZ³═C(Z³)₂, —C≡CH, —C≡CSi(Z¹)₃, —ZnX⁰ or —Sn(Z⁴)₃,
X⁰ is halogen, and
Z¹⁻⁴ are each independently C₁₋₁₀ alkyl or C₆₋₁₂ aryl, each being optionally substituted, and two groups Z² may also form a cycloboronate group having 2 to 20 C atoms together with the B- and O-atoms,
wherein at least one of R²³ and R²⁴ is different from H.

32. The compound according to claim 7, wherein Ar¹⁻⁴ are selected from:
(a) formulae D1, D7, D10, D11, D19, D22, D29, D30, D35, D36, D37, D44, D55, D84, D87, D88, D89, D93, D94, D106, D111, D139, D140, D141 and D150, 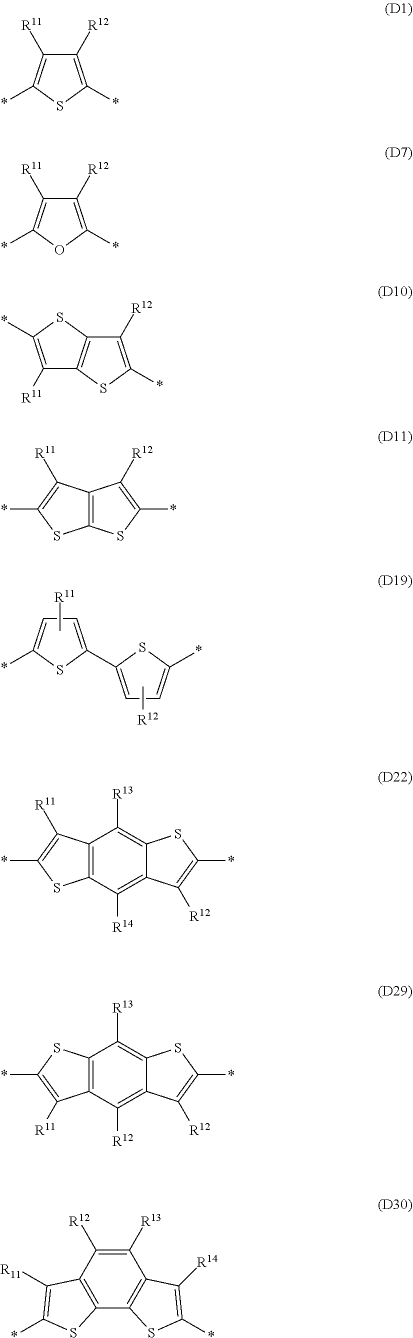 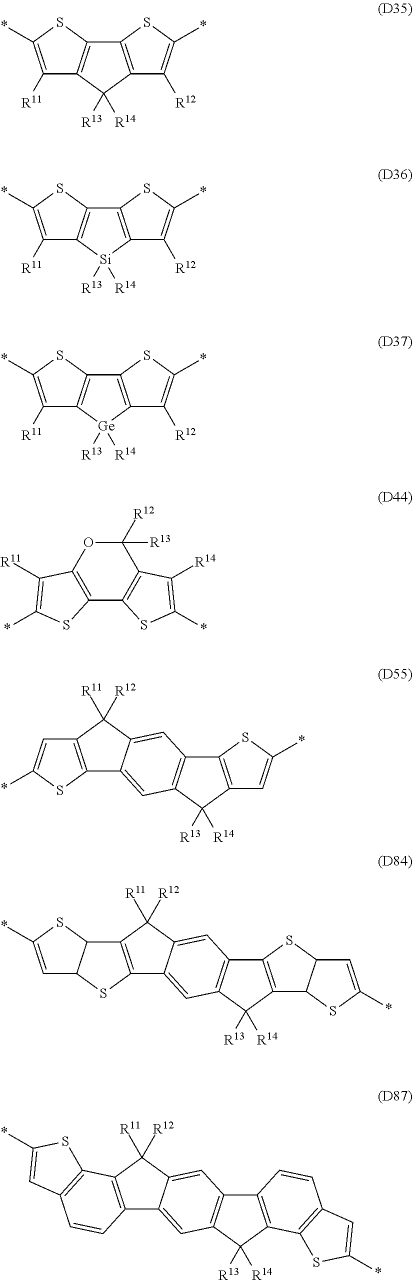 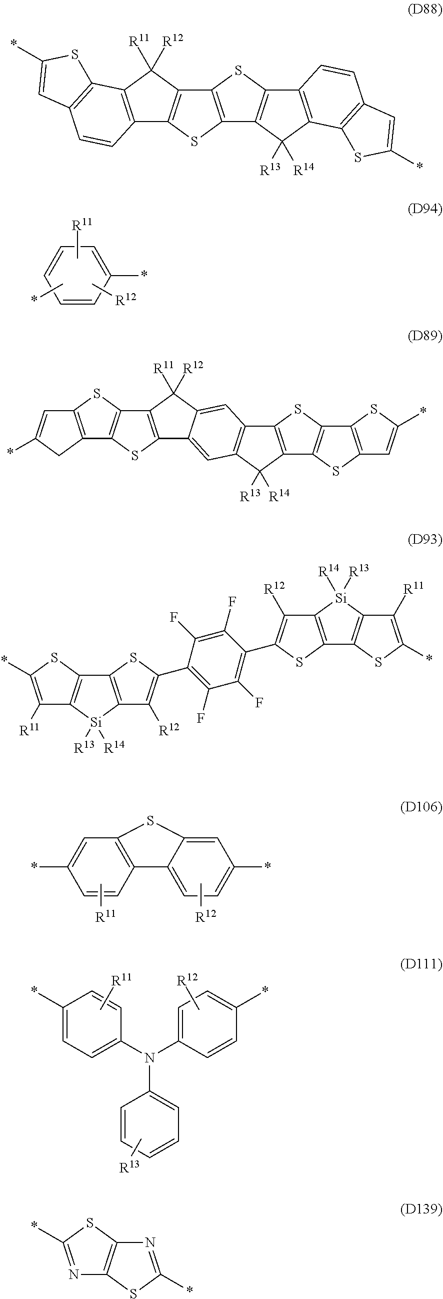 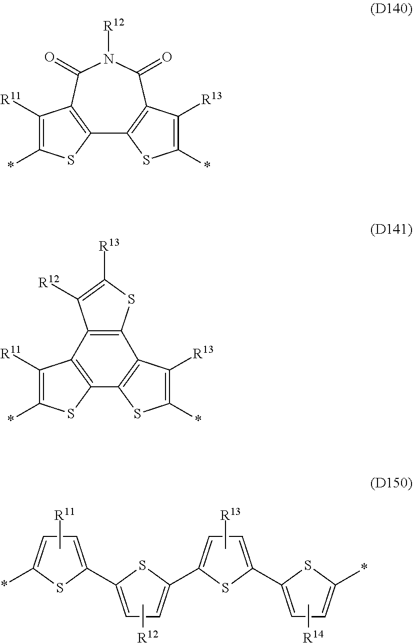
wherein
R¹¹, R¹², R¹³, and R¹⁴, independently of each other, denote H, F, Cl, —NO₂, —CN, —NC, —NCO, —NCS, —OCN, —SCN, R⁰, OR⁰, SR⁰, —C(═O)X⁰, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO_{2R}⁰, —OH, —NO₂, —CF₃, —SF₅, or optionally substituted silyl, or carbyl or hydrocarbyl with 1 to 30 C atoms that is optionally substituted and optionally comprises one or more hetero atoms,
R⁰, R⁰⁰ H or straight-chain or branched alkyl with 1 to 20 C atoms that is optionally fluorinated,
X⁰ halogen;
(b) formulae A1, A6, A7, A15, A16, A20, A36, A74, A84, A88, A92, A98 and A103, 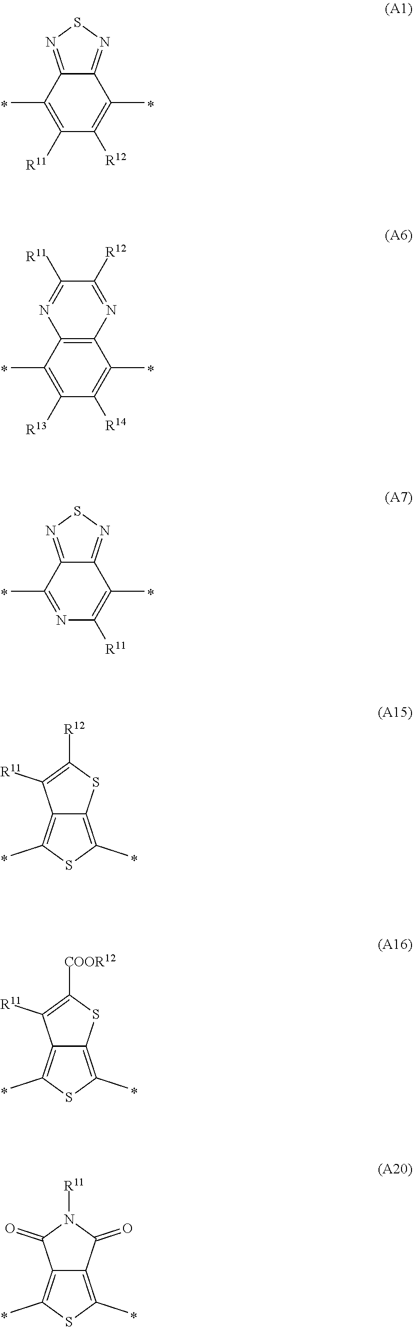 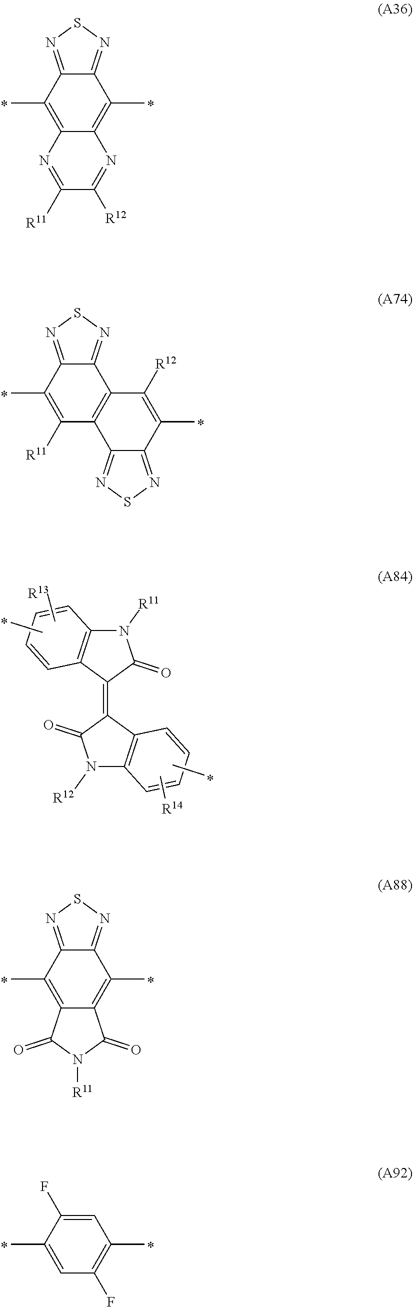 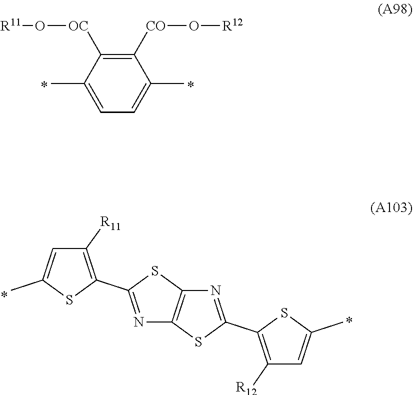
wherein
R¹¹, R¹², R¹³, and R¹⁴, independently of each other, denote H, F, Cl, —NO₂, —CN, —NC, —NCO, —NCS, —OCN, —SCN, R⁰, OR⁰, SR⁰, —C(═O)X⁰, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO₂R⁰, —OH, —NO₂, —CF₃, —SF₅, or optionally substituted silyl, or carbyl or hydrocarbyl with 1 to 30 C atoms that is optionally substituted and optionally comprises one or more hetero atoms,
R⁰, R⁰⁰ H or straight-chain or branched alkyl with 1 to 20 C atoms that is optionally fluorinated,
X⁰ halogen; or
(c) formulae Sp1, Sp2, Sp6, Sp10, Sp11, Sp12, Sp13 and Sp14, 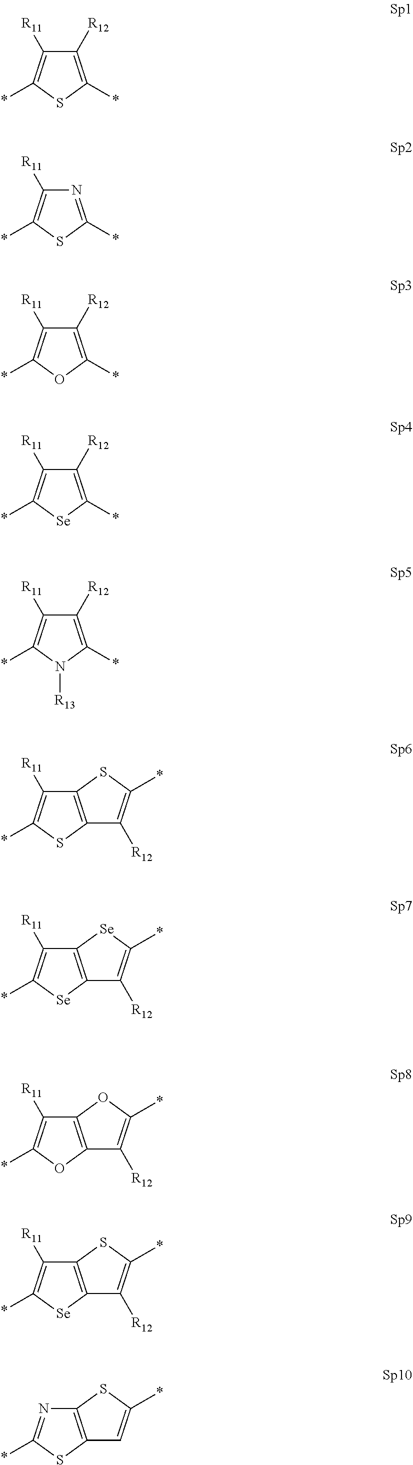 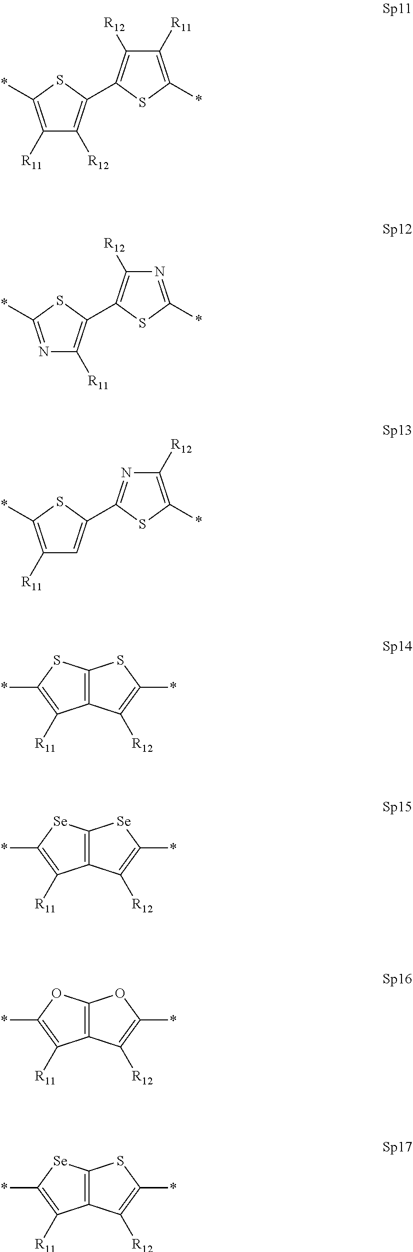
wherein
R¹¹ and R¹², independently of each other, denote H, F, Cl, —NO₂, —CN, —NC, —NCO, —NCS, —OCN, —SCN, R⁰, OR⁰, SR⁰, —C(═O)X⁰, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO₂R⁰, —OH, —NO₂, —CF₃, —SF₅, or optionally substituted silyl, or carbyl or hydrocarbyl with 1 to 30, preferably 1 to 20, C atoms that is optionally substituted and optionally comprises one or more hetero atoms,
R⁰, R⁰⁰ H or straight-chain or branched alkyl with 1 to 20 C atoms that is optionally fluorinated,
X⁰ halogen.

33. The compound according to claim 16, wherein Ar¹⁻⁸ are selected from:
(a) formulae D1, D7, D10, D11, D19, D22, D29, D30, D35, D36, D37, D44, D55, D84, D87, D88, D89, D93, D94, D106, D111, D139, D140, D141 and D150, 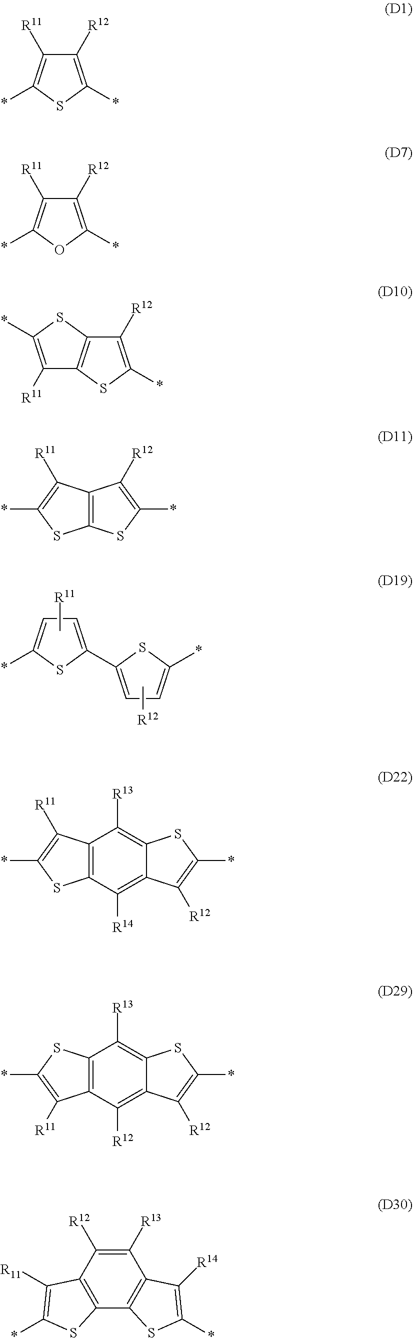 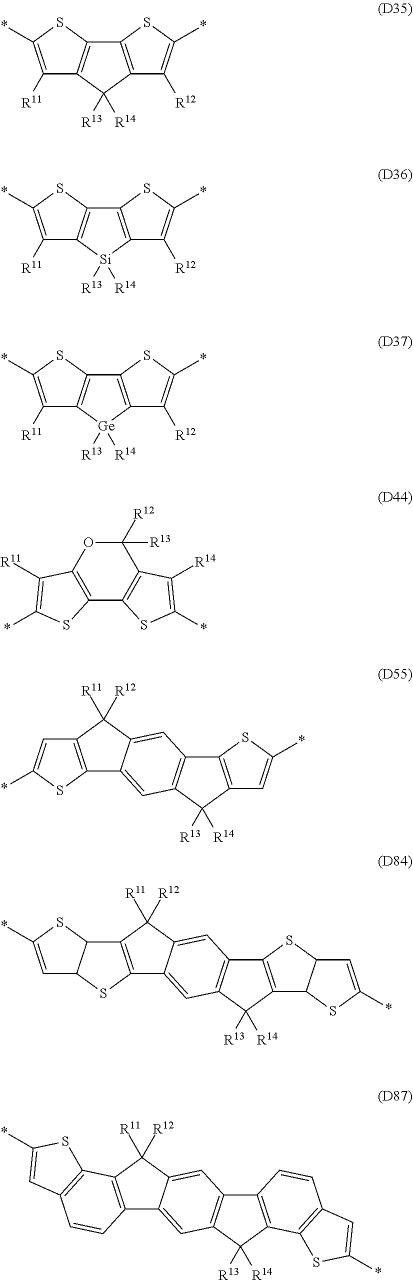 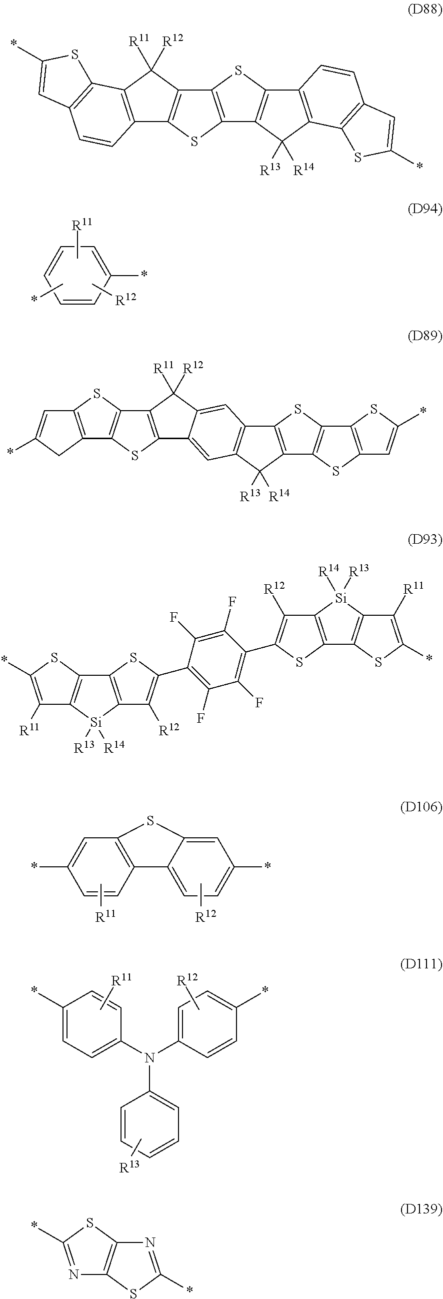 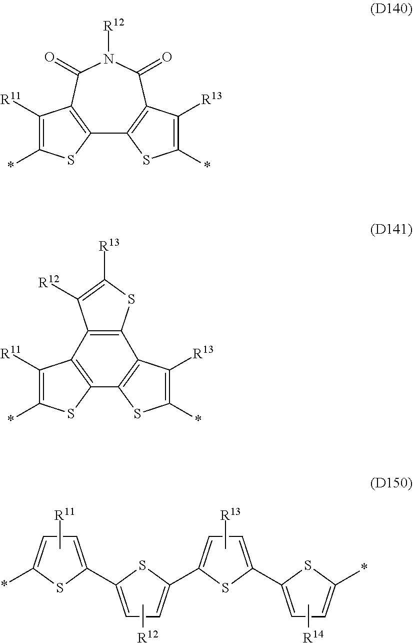
wherein
R¹¹, R¹², R¹³, and R¹⁴, independently of each other, denote H, F, Cl, —NO₂, —CN, —NC, —NCO, —NCS, —OCN, —SCN, R⁰, OR⁰, SR⁰, —C(═O)X⁰, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO₂R⁰, —OH, —NO₂, —CF₃, —SF₅, or optionally substituted silyl, or carbyl or hydrocarbyl with 1 to 30 C atoms that is optionally substituted and optionally comprises one or more hetero atoms,
R⁰, R⁰⁰ H or straight-chain or branched alkyl with 1 to 20 C atoms that is optionally fluorinated,
X⁰ halogen;
(b) formulae A1, A6, A7, A15, A16, A20, A36, A74, A84, A88, A92, A98 and A103, 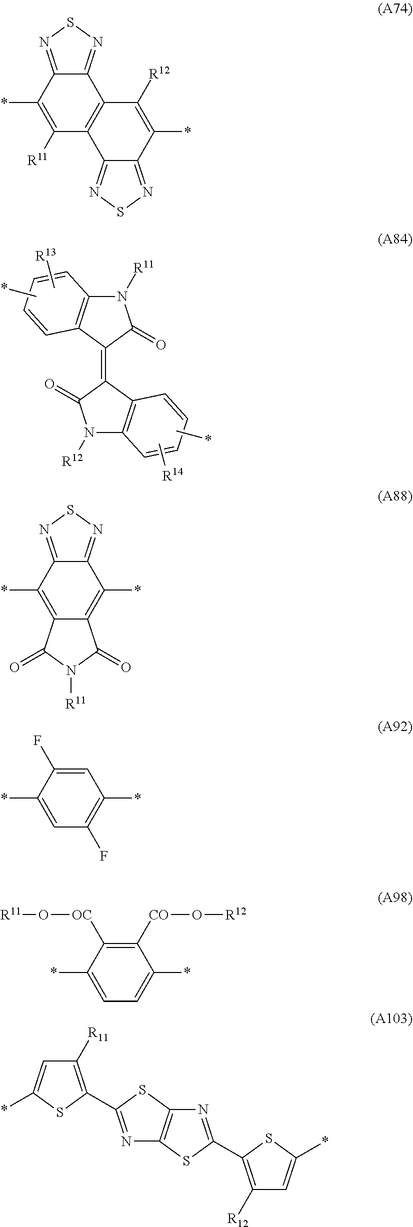
wherein
R¹¹, R¹², R¹³, and R¹⁴, independently of each other, denote H, F, Cl, —NO₂, —CN, —NC, —NCO, —NCS, —OCN, —SCN, R⁰, OR⁰, SR⁰, —C(═O)X⁰, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO₂R⁰, —OH, —NO₂, —CF₃, —SF₅, or optionally substituted silyl, or carbyl or hydrocarbyl with 1 to 30, C atoms that is optionally substituted and optionally comprises one or more hetero atoms,
R⁰, R⁰⁰ H or straight-chain or branched alkyl with 1to 20 C atoms that is optionally fluorinated,
X⁰ halogen; or
(c) formulae Sp1, Sp2, Sp6, Sp10, Sp11, Sp12, Sp13 and Sp14, 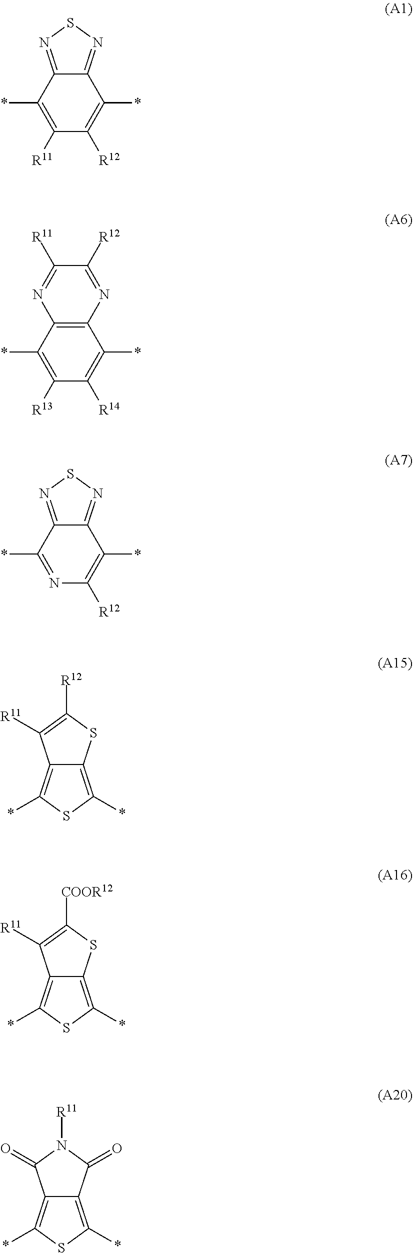 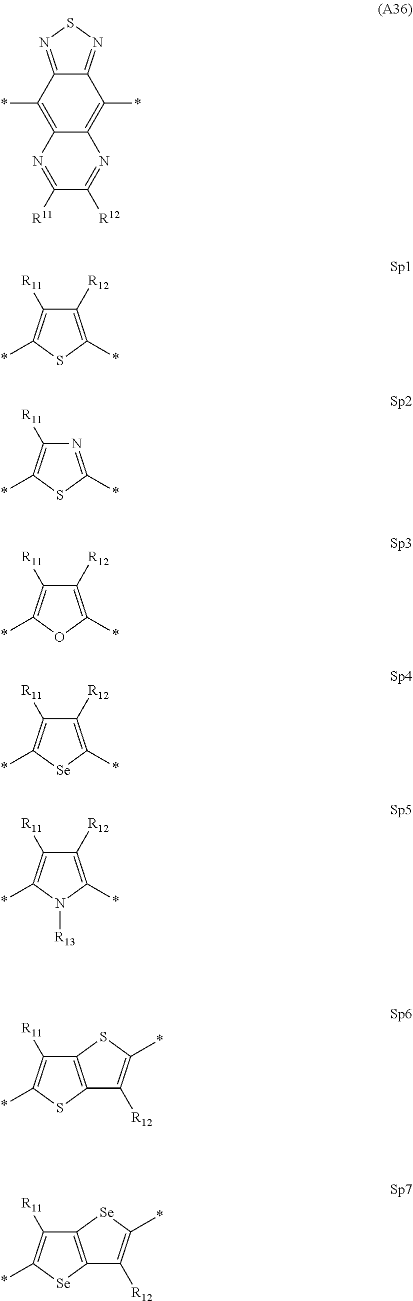 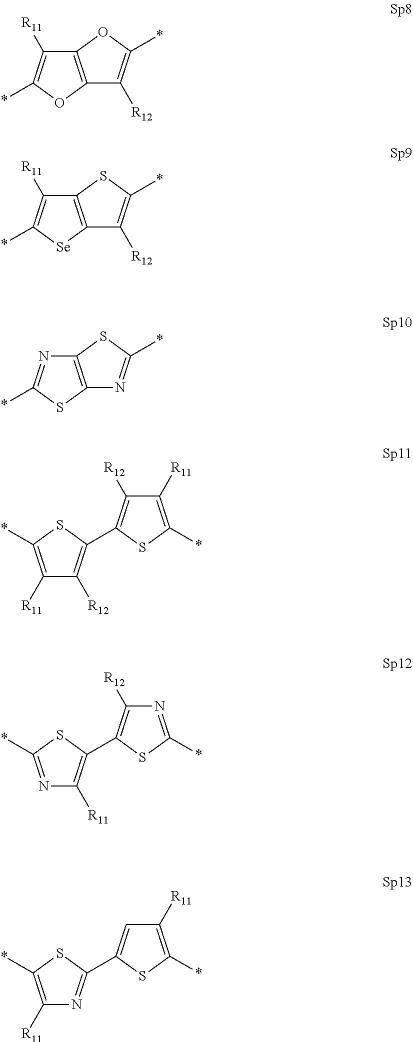 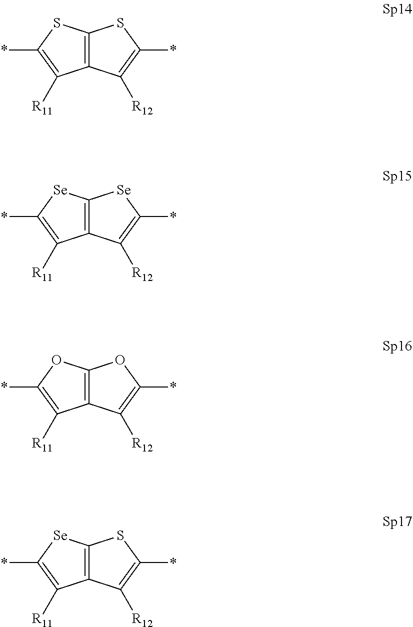
wherein
R¹¹ and R¹², independently of each other, denote H, F, Cl, —NO₂, —CN, —NC, —NCO, —NCS, —OCN, —SCN, R⁰, OR⁰, SR⁰, —C(═O)X⁰, —C(═O)R⁰, —C(═O)—OR⁰, —O—C(═O)—R⁰, —NH₂, —NHR⁰, —NR⁰R⁰⁰, —C(═O)NHR⁰, —C(═O)NR⁰R⁰⁰, —SO₃R⁰, —SO₂R⁰, —OH, —NO₂, —CF₃, —SF₅, or optionally substituted silyl, or carbyl or hydrocarbyl with 1 to 30 C atoms that is optionally substituted and optionally comprises one or more hetero atoms,
R⁰, R⁰⁰ H or straight-chain or branched alkyl with 1 to 20 C atoms that is optionally fluorinated,
X⁰ halogen.
